SEQUENCE LISTING (6 sequences)

SEQ ID NO: 1 - DNA, 258 bp, Homo sapiens
cagcggagtgatggcaagcacgacttccgggtgtggaatgctcagctcatccgctatgctggctaccagatgccagatggcagcatcagaggggaccctgccaacgtggaattcactcaggtacccggcccagcctcagccaccggccattggggcggggagccccgtggtgagcgagtgacagagtggagcccagaggagacacgcagcccgggcttacagactcacagggcccgtcttgttccccagctgtgcatc

SEQ ID NO: 2 - DNA, 24 bp, Artificial Sequence
  Chemically Synthesized Forward Primer
cagcggagtgatggcaagcacgac

SEQ ID NO: 3 - DNA, 24 bp, Artificial Sequence
  Chemically Synthesized Reverse Primer
gatgcacagctggggaacaagacg

SEQ ID NO: 4 - DNA, 136599 bp, Homo sapiens
gatcaggtaaattttgtcattggatggaacacatggccattttacactcatggctcacattttcaaaagctaacataggaaggagaatacctacagcatgctcatcagagacaggtttttcaacagcatcttataatttagctaaaaaatcagggtataattcagtgtgaccttgtttaaccgtagtaaaataaacaggagcttggcctggggcacataatttatcccaagctctcatacacacctttgttgcttgttctgtggtaagagcatcaaagtttaattgggcataagtatcagagaaactatcagagcctgtgagctgagcctgagtaattagaaagctaatagtccaatttagctgggcctgcaaacgggcctcctctgaccaccaggtatgaaattgtaaatgctgagatggggttaaaacagctttttccaaaaggtcccagtctaaaggaagaaaaatgacctcagtacaaaaagttagtaataccattttaacataaggagaagtagggccatactgagtacaagcatccctaaattcttttaaaaaggtaagattgagaggtacataatgacacatttgtaccccttggaagttgggaggttctagcatgactggataagcccacgcatctaatccatttgtttgtttattttggcataataaacattgcactgaagttttaagagtaggcatctgagacagagttggcatagatgtgacaggaaaagcatgtgtagattagggaaactgagggtgagggggtcagaccagaatgagagcatgagaaatgggcattgggggagcagaaggagcagaagtatactggtgattactggcatccctgtgtgaagaagggtacagagaagcaggctgagtggatggcaaagtgactggttgaggaactgaaatgaccagagggtgatgggctgcagtggatgggggagggcctgaataattataggtaaattgtagtttggtcccagagccattagctgactccagaggtttgaagagagaagaatttgcatatatatggtcccgggctgtctgagttggggccatgggagctacaagtaccagctctttgtgaaaagaaataaggtcatcaaggggtgacattaagccaaagtcactggagttagatattgaattgtcagcatcattaattgggggaagagtaggcgaagggagaggctgatcagttaacaaaggctgtgtaggagaggaaggttgaggaagaggtggagggtcatcagattcagaaaattgtggtaactgtagggagtcacgggattggtatgtcattaggatggcacatgccaaggcccaatcaccccaaacagtgatgggaacataatttcctgttgagaccagttcctggaatgttgtgccaacactatcccatagttctacaactaaggttcctttttcaggaaactaaggacagtgttcttccaccgccctgaatagagtgaccatattttccatggttactcagactctgccctgtttttacaggagtttaatatagcagaggtaagcatgaagtttagactccatgtggcccatagttaacctggaccatacacagactactcaccagttgtcagggagtcgaacaagtgtttccttggactgaaccaatggcatttctccccacctatcaaagggaattgggtttgcacatgcacttaggaaaaaagaaaaaccacactgggtgccagatatcaggggaaccagcccccagtatttcaacgtaggttcttttctattttccctaagtgtcagccagtctgagaaataaaaagggtgcaaaacagagaaattttacagctgggcctctgggggtgacatcacatgttggcaggttctgtgatgcccacctgagccacaaaactagcaagtttttattaggcatttcaaaaggggagggagtgtactaatagggagtgagtcacagagatcacatgcttcatagggcaataaaatatcacaaggcaaatgggggcagagcgagatcacaaggccaaggtgaaattagaattgctcatgaggtttcatgtcccactgtgcacacattgtcattgataaacatcttaaccagaaacagggttcgagaacagacaactggtctgattagaatttcaccaggctggaatttcccaatcctagcaagcctgcaggcattgcaggagactagggcatatttcatcccttatctacaactgcataagacagacactcctagagtggccattttagagacctccccctgggaacgcattgttttcccaggactgttccttgctgagaaaaagatttcagtgatatttctcctattcacttttgcaagaagagaaatatgactctgttctgcctggccccacagggagtcagaccttatggttatctctcttgttcactgaaaatcactgttatcctgttcttttaggatgcccagatttcatattgttcaaacacacatgttttacaaacaatttgtacagataatgcaatcatcacagggtcctgaggtgacatatatcctcagcttatgaagatgatgggattaagagattaaagtgttgaacaatgtaaacaatcatgagaggaaatacgctgttggggatgatgctcagtttgagatggacatctgactcttctgcaaggattagaagaaaagcagcaatgtagatacttgtgtgcacatgatttggccaataggatcaaccatgaaagacagatgatgcactaccagcagtccccattacagtctctacctccccctcttcttctgggtggcaagtgatgggaagatgatcttcatctgaccacttcttctacctgtctcctgttcccctttccggttaaacaggatagattgaaggcctttggtgtatttctgtagagctaagcagcccttagaggaaaacaaataaattggatttcctagttgttttaaaaaaaagaagaagaagaagattaaagtaaagacaggcattggaaattataagagtattgattggggaagtgataaatgtccatgaaatcttcacaatttatgttcagagattgcagtaaagacaggtgtaagaaattataaaagtattaatttggggaactaataaatgtccatgaaatcttcacaatttatgttcttctgtggtggcttcagccaatccctctgtttggggtccttgacttcccacaacaacatttagttgattatatcaaggcctttgacagtatcaaaggtaatctgcataaagctactctgctggcacaagccatggcaggactgagagtgggtaaaggaaatgctctgtttcctggagcttgtttcagctgtgggaaacatgggaatacaaagagaatgtagaaaaaatcagagggtccaaatgcccagtgttggaaaaaagaaaactgctgaaccaggtctatgcccgaagtgtaaaaaaggaaagcattggccaatcagtgtcattctatatttgagaaggatggaaacccagttttggtatatgccatgagggccccgccccaggccccattccaaaccagagcattcgtggctcaggccactccctcacccctgtacaatgcttgttccccaccacagctggtggtgccacagtagatctgtgttgcaccagggctgtgagtctcctccctggagagccaccgtagaaagtaccaacaggagtctgcagacctctgccagcaggaaaggtaggactacttctgggaagatctagtttaaatttaaaaggagtgcaggttcatacaggagtaattgacttggattgtaatggggaaatttgaatcattatatctatctccattccttggaaggcagaactgggagagcatatagtgcagcttctgattttaccatatgtgggactaggaaaaagtgaaactaaaagaacagggggatttagcagtacaaataatcaaaggaaagccacctcctgggtaaatcaaattactgataagtgttctacctctgaaataactattcagggaaagaaatttaaaggtttggtagatacaggagcaaacattgcaatcatttatctatagcactggccatccatgtggccaattcaacccactcaatttaacatatttggagttggtaaagcccctgaggtatatcaaagtagttacattttgtattgtgaagggcccaatggacaacctgggactatttaacaaattataacttctgtacctataaatttatgggggagagatttattacaacaatggggagcacaagttttaattcctgagcaattatacagccctcaaagccaacatatgatgcatgaaatggggtatgtctctggtttgtgcgtaggaaaatatttgcaaggtttgaaggaactacttcaagcagaaagacaaagttcctgccaaggcttggataccatttttgatggcagtcattgttaagcctccagagcctatctcattaaaatggctgacagacaagccaatatggatagaacaatggccactgaataaagagaaactggaggctttaaatgaactagttaaagaacagcttcaaaagggacatatatttccaacactctccccttggaattcctcagttttcataattaagaaaaaaatcaggtaaatggagaatgttaactgacttaaaggccattaattcagttatacaacctatgggggcattacagccaggactgccttctcctgctatgattccaaaaaattggcctttaacagtcacagatttaaaagactgtttctttactatcaccttagctgagcaggactgtgaatggtttgcatttatgattcctgtggtaaacaacttgcagcttgctaagcattttcactggaaggtgtttccacatggcatgttaaacactccatccattttccagacttacgtagggcaagaaattgaacctactcataaaagattttcacagtgttacattattcattatatggatgatattctttgtgctgcccccacttgagaaatattactccaatgttatgatcacttgcaaaatttctcatgccagtttaattatagctcctgacaaaattcagactactactccttactcctacttagggaccttagtaaatgacactacaatagtgccacagaaagtaaccatatgtagggatcaattgaaaacattgaatgactttcaaaaattactaggggacattcattggatatggcccattctaggcattcctacttatgccatgagtaatctgctttctatccttagaggagatcctagtctcacaagccctcagaaattaacaaaagaagctgaggcagagcagtagctaataaaaggcaagtccataaggcccaaataaatagaatagatccagagaagactcgagatttgctaattttttcagctcagcattcagctaaaggtgttattgttcaagagcaagatcttgtagagtggctttttcttccacatactaattcatggactttgactccttatttggatcaaatcgctactatgataggaaatgggagaactcagattgtttaattacatggatatgatcctggaaaagttattgtccccctcatgaaggcacaaatatagcaagcttttataaatagtcttacttggcaaatccatttagctgactttgtgagtattctcgataaccattttcctaaaatgaaactctttcaatttttgaaattaactaattggattctccctaaaataactaaattcaaaccaattgaaggtgctgagaatgtcttcacagatgggtctagtaatggtaaagcctcttattctggatcaaaaggtaaagtttttcagacgccctatacttcagctcaaaaagcagagcttgtagctgtaattgaggtattgactgcttttaatatgcctattaatgtgatttattattcttcatatgtggttcattctacagagttagttgaaaatgctcagctatgattccacacagatgatcaacggatgactttatttacccaattgcaagagcagttagaagtagaatgaacccttttttacatcactcacattagagctcatacacctcttccaggacctttgactgcaaggaatcaattggccaatcgcctagttgctactgcaatatctaatgctagacactttcacaatttaatccatgctaatgtctctggtctcaaacactgataaagcattacctggaaagaagctaaagctattatccagcgatgcccaacttgccaaatggtgcattcctcatcttttacaagaggagttaatcatcaaggattggaacctaattctctttggcaaatggatgtcatgcatgttcccgcatttgggagactagtttatgtacatgtatgtgtggacaccttttctcactttgtctgggctgcatgccaatcaggagagtcttctgcctgtattaaatgtcaccttttgcagtgctttgcggtgatgggcattctagcttccattaaaacagataatgccccaggccctactagccaagctctagctacatttttctctatatggaatattaaacacattactgatatcccatataattctcaaggacaagccatagtggaaagaatgaatctctccctgaaacagcagttgcaaaaacagaaaggaggagacagaaactataggactccatacatgcaactaaatctagcattattaactttaaattttttgagcctgcctaaaggctgattgttattagcagctgaacaacatttacagaagccagctacaaaagcagaagtggaaaaatgggtttgctagaaaggcccaataacaaaagtttgtgaaacaggtaaaataataacatgaggcagaggttatgcttgtgtttctccaggaccaaatcaatagccaatttgggttccatcaagacatctaaagccctactatgagccagatatcaaggaaaaagttttgggaggatccaaaagaccaactggtggcagcgttgtccaagttgatgctgaggaggaccccaactgtcacgagcaatccccgtctaacacagccacctacctggggacagatcaagaagctgtcgcagatggtggaagaaaacctgaggaaagtgggacaaccagtcacaatgagtaatttactgatagctctgatagctgtgattaccattgccataagtattccttcagcaagggctgacacagagaacaattatacttactaggcatatttaccttttccaccacttctatggcctgtaacttggctggaccccccattggaggtatacactaatgatagctcttggatgcctggtcctacagatgatagaggtccatctcacccacatgaggaaggaactgttatgcatatttctttaggatttgaatatctgcctatctgtttaggaaaggccactagttgcctatccccttgctatcaatcttggctggcaataatgcctggacataatcactctatgatgcagttttacatgctttctggtctcagtatttaccataatgaatctgctcctgtaattgaggcataccaccctcaagaacctatttgtaaacagaattggacctggttagaaataatgaacatacttgtttgggaaggttgcattgcagaggtgctgcacaacaggcagaggtgctacacaatgattcctatggaatcattattgactggtcccctaaggggatgtttagcatgaattgcatctctcagtctgcgtgtgatggccacactatgttcagctggtctgaacaaaatgatcagttggtagaaatgataagaagtatggcaagatttcctattatctggaaacgtggcggtatggtggcacctcaacttcaaatgatcctgataagatcaagatttgggaagaaaaatatactatgccattcacattgtaatcctaattacattttagaacttgttgaaaacaatactatttggatacaaagttgtgttcatcctgcttttctgttggtaatgggcaatctaaagtttggcatccctaattatcatgtaactttccaggaatgtagattgttctcttgtgtgaactctccttgtttaatactaatcattctattctagtagagagtgctcaagaaggagtatggttactggtgaagctttcctgtccttggaaagcctttccctctgtacatattattactgaaattcttcagaaaattctgagacactcttgacatttcattgatactttaatcttgattattatgggaatgatcgctgtcacagctactgctgcagcagctagagttgtgctacattcatcaatgcacagcagattttgtaaataaatggcaaaagaattctactctgctgtgtaactctcaaactaaaatagaccaaaagatagttaatcaaatcaatgatctccaacaaactgtaacgtggctgggagattgagcagctagtttgggatatagaatgcagctaaaatgctactggaatacttctaacttttgtatcactcctcacccttataatgaatcagagcatgaatgggaaagaataaaaaagcatctagaaggacactctacaaacttgtctttggatgttgcaaaactaagagaacaaatatttcaagcatcccagggacacctgaccttaatgccaggaactggagtgcttgaaggagttacagatggattggcagccataaatccttcaaaatggataaaaacacttggaggctctgtgatttctatgacggttgtgttattaacctgtgttgtttgtctttgtgtagtctgcagttgcagatcccgactcctgcaagaagtagctcaccttgataaagccgtctttgtatttattgccctgaaaaaacaaaaagggggacatgctgggaacaggccccaagactggccataaacagatttctgcagcactgtgatatgctcatgatggctatgatgctcacgctggaggttgctggtttaccagaatgagggcaaggaacatctggcccacccagggtggaaaactactcaaggcattcctaaaccacaaacaattgcatgagtgatctgtgccttaaggacatgtccctgctgcagataacaagccagagcctgtccctttgttctctgtgaagaatactttcagttaatctataaactgcagaaatgatgtttatcacaggcttactgtcaataaataggtgggtcaaattctgttcaagactgtcaggtctgaaggctgttagccccctgttcccactttgcactctatttctgtgcctgtgtttttattcgtctagtgctgctgggttgcggtctccatgactgagctggtttcagtgcccatctctgctaaaaattacaaaaaattagccagacgtggtggaagctgtaatcctggctacttaggaggctgaggcaggagaatcacttgaactctggaaacagaggttgcagtgagccaaggtgttgccattgcattcctgcctgggcaacaagagcaaaacatcttctgaaaaaaaaaagaaagaagaaagaaagaaagaaagaaagaagaaagaaagaaagaaagaaagaaagaaagagaaaagaaaagaaagaaatagttgctaaattttctcaatactaaaaatatttgcaactgggtacagtggcttacgcctgtaatcccagcactttggcaggccaaggtgggtggatcacttaaggtcaggagttcgagaccagcctggccaacatggcaaaaccccatctctactaaaaatacaaaataaattagtagggcatggtggtgcatgcctgtaatcccagccactcgggaggctgaggcacaggattagagctgccaacaagcccagctaatttttgtatttttagtagaggtggggtttccccatattggccaggctggtctcaaactcctggcctgaagtgatccacccaccttggcttcccaaaatgctgccacccttggcagtctgaatttttaaaaagtaatgaatattcttaatatcttttaaaaatacagtgatatgaaaataagggaaaacgtttaaagattattttgttctttacaattctcttggttctgttcttttttgattagttttgggattaaatttattgggagaggtccaaaatgatgaaatttgaaaataagctctattaggtgcactattaagtagaaagagcctctcaagttttctcctaggctctttgaagttattttgtttgggaacttttttcttcctaattctgcctcataagacaaagtactgggaggtacaaaggatagtgatttaatggcactgttttcctaacactgaattattttcatttgaaagaagttgatcactcttcagaaaagtaattgtaaagttatttttacccttggttcatttgatttcttaaacagcagttttcccgacagaaatttacagtgtgatgacaatacataactggttcagtgagatgaaaatccaataatcagcttatgttgggactggatgttgatttattacaggattatacacatgaattccataaaaccaaagcaaactttatggcaatatgggaaagggagaatctcatgggatcagtacaaaaagatattgagtaatttgccttttatattattttgtagaatatttattcagatttggctttaggatatgttcacagttaattacaaattgagtggcagaaccatggttgagatttcaggatttccaattcccatttcactgtcttttaagcattgctgctacaatgtcatcctaatctacttttccatttttatctcatgcttccccttattttatcattggctcctgccaaccttgactttcctgtcttaattccaagtccagcttagatggtatcatcatggttccctgatctcttaccacacaccctaagaattataatgttacctttatgatcctctgcagatgtgtcaaatacatttttttaaattacaacattatcagtaactgaagctttccatgtttctcttcctgattatcacctatctctgccccctcccttgtcccaacaattatatattataaactataatacatatacaaactcatagccttaatgttaaattatttaaggactgtcaacatttttatgctgatctcaatctataattttaaatattttttaggtgaggtcatttcctctctcctttaataatctattctagcatttcacacacatttttggaaactttttcacaactgaacactactgacaatgttaagtcccatgggcatctcactgaagctgaacactttccaatttggagctatctaatgatacctgtcatcgtctcacttgtgatatattatgtgataagtttcataaactatgatatacattgatcataaactatatattatgtatactctcagaaacacttccctagagatcctttatactgttgtaacaggttcaacagtaagacagttgtaagactttttagtccttcaggacatgtaacccttttgtttttgtttgtttcttttttgagacagagtctcaccctgtcacccaggctggagtgcagtggcacgatcttagctcactgcaagctccacctcccaggttcacaccattctcctgcctcagcctcctgagtagtggggactacaggtgcccaccaccacgtccagctaattttttgtatttttagtagagacggggtttcaccatgttagccaggatggtctcgatctcctgacctagtgattggccctcctcagcctcccaaaatgctgggattacaggcatgagccactgcacccggcctcaaggcttttgtttaatttccatacagtccatttacatatacattttgaggaatgcattgagaacctgtgtaggggatggcctttaatgtatagacattgagcaccccccacacacaatgtgtgggaatcactagaatgcaacccattgtaataagttgctttttgaaaaatggccataaaagatatgtttgtctattccttaaactagctcctgagtaccaactctgagttagtcacagtagaatgtagaaatgaaaagaccaatgtggccctgtaggagtttataatccaggtgctagaggaaagtaaggcaggtactcaagtttgtataatccaaggccgaatatgattagtgagatgagatgataatagtacctaacattgtgtgtgtgtgtgtgtgtgtgtgtgtataaatttacacagtattttttatgtattatcttttttggcttcataataatcttttggttaacatagaaattatcagtctcattttacaaaactaagtattgagcacacttgtctaagtgaagcggctatgaaatgctggagtatagatttaaatccaagtcatcagtcccccaagtccagtgtgttcttggttatatcattctgctttcttcggtggtgttttctcctgtgctttgcacagtagccctgcgtctcagccagtatttctgttttagcagcatgactctcttttgtcttattgtagtatttgcatggttaacttgaattttaaaaatttgaggccaggcacagtggctcacatctgtaatcctagcactttgggaggccaaggcaggtggattgcttgatctcaggagtcctagaccagccaggggaatgtggtgaaaccctgtctctaccaaaaatacaaaaattaggtgggcatggtggcatgctcctgtactcccagctacttgcgggcctgagacaggaggatcacttgaacctgggaggttgagaaggctgcagtgagccgagactgtgccactgcactccagcctaggtgacaaactgagacaccgtcaaaaaaaattctttgcttgatttcagtgtttcttaggcttcatttggtgggctgcatgcactattgtgaaggaaaacattccattccaactgctttcccaccccatgtgctagatactaaatcagtgctacattttacttccacctggagaaaatgaagatatcagagataatagacaaaggaaaagtaagattttttgtttgtttgtttgtttcagaatgttgagttattttggtgaaaaacatagtaaactgttagaactaaaatagaggcataatatttatctgtttataatagatatcttttttttgagatggagtctcactctgtcactaggctggagtacagtggcatgatcttggctcactgcaacctccacctcctgggttcaagccattttcctgcctcagcctcctgagtagctgggaatacaggcacatgccaccacacccagctaatttttgtatttttagtagagatgtggtttcaccgtattgcccaggatggtctcaatctcctgaacttgtgagctgcccatctcagcttcccaaagtgctgggattacaggcatgagccaccatgcccagccaatagatatcattttttagggcaaaaggaaaaggattatgcacttttatttttcattcacagctatgaagaggctcaatttatctactttcaagatcactagggtcagtcagttaatctgattttgtattctctgccaattttttttttttttttgatggagtctcactctgtcaccaggctggagtgcagtttcatgatcttggctcattacaagctccacctcccagtttcaagcgattctcctgcctcagcctcccgagtagctgggagtacaggcacgtgccaccatgctcagctaatttttgtaattttagtagagatggggtttcaccatgttggccaggatggtcttgatctcttgacctcgtgatctgcccacctgggcctcccaaagtgctaggattataggcatgagacaccgcacccagcccaaaaatgtttttaaagtttctcttttgtcccaggtcatataaaagtcagtctggagtaaacaacagaagaactgagctatttttgaaagaacacgaccaccttcaaaagtaggtattattgaatgtatgtgcattatgtggttttccatgtttattaatttatttacttatgtaacaaacattttattatgtttatcatagtaagtgctttatccattctaattcattttattcttctgactctttttaatagaaaagaaattgcaactattctgaccataatgaggaaaggccttgtgctcttgagtatttcacatcacttaattgttaccttaatatgttttttttattgccttagtgtgttcttttcctcagcaagtcttgctttaaagcttttgtccaatactacaggttattagtaaaaataagtctgggtgtggtggctcatgcctgtaaacccagcactttgggaggccgaggcaggtggatcataaggtcaggagttcaagaccatcctggccaaaatggtgaaaccccatctctacaaaaaatacaaaaaaattagctaagcatggtggcaggcacctgtaatcccagctactcaggaggctgaggcaggagaattgcttgaacccaggaggtggaggtggcagtgagctgagactgcaccacagtactccagcctgggtgacagactgagattccatctcaaaaaatacaaataataataataataatcaaataatgacttatgtttgaatggcctgttaccacttatatataaagaagccttataaagagacattggatcatataccaaatttttaaaaagtttttattttccaggatttcagaagaaaatacttaaatacttggatgaactgtatatatatcttaaaacaagctgccatagtctgcttcattatttttctacttttctctgaaatgtacaggtttctttgtccttggatttatgttttgttgtagaacttacagatcagaaaattacttatctctatggacttagcctataagcacttgagcagcttgtatcggtgagagttaagtgttaggaattatgttgacatttgctactgaatgtcataaattagtcacgtcttatgggcttaacatgctgttcaatttagttttctttaaaagtcaccactttaaatataagctgttgaaatcaaaacacagaaagatccagacctttttagaaacctgggtgctcccagtgttggaactagttttctgaatctgttggatgttattgatttgcacaagggggcaggctcttctagagcatgtttagtttgtagtattcatagctttggtagttcttttggtatttgacaaagaccaccataaaaagaattttgcattgtatccaaaattatattaatctattctgggggaacacagtaatttaagggatagcatattctagtagcataaaatacagattagattgcataagtatctctatatttactgcctcaaaagaaacagaaatgcagtagttgactttgacaagaatgatttgatgctgagaagagtctatcagttattatatacatgaatattctttatttcatgacttcagaacacatcacatattaaaacttttctatgaattttgcagaaagttttgcatttgctttaattattcaaggtaaatccttgaggagtttaataccattcattttacaggaaaatgaagcaattcaagtatacacatctttaataatttgtttagggttacaatcatggtgagaagatggagatcaaaaggtatttttccatttttgtttgatcaaagtataaagtctcaattttacatataccatactttatatatattgtatagtttatgtacatttatttcaacattcttgtgaagaagtttgtgtgtaccaattctatgcagagagaaactgagacaggatgaaattgaatggaataagagcttctggaattagagaagttgttgagtctgaaaaccagggcttagtcattgttctcaaaatgagaacacaagcttccaaatactttttttagtcaattttgttttgtaaattaaaactgctggctagataataaaataacattaaaattattaataaaatatgtttataaaataatgtaaaaaactttagtttttcaatgttattgtatttcatttgctaagtgctttgaggaagaagcagcaagatatttttgaaataacaaaagctattaatgtgcttttgtcatagtgctcattttaaaaagtatgatagttcaacaacataaaaagaaaatttaaatcgcacattaatacaaattttgtaacttttttactctttagtagattgcacgtagttttcccatgtagtttgattttttaaaaatatatggtgcatatattgcataatacagataaaccataaattatgtaaccatttccctaatgttgaccttttggattattttctgtttttactattatacatagtaaattctttgcatgtatctatcattattttcttataattcctaaattagaattgcaggattaaagaacatactcatttgcaaagtttttcctataatgttcccaaatttccaagtttgattttttgcagtagggtcagactttttatggctacaactatgtctaagcttgtgcttcttcacaaatattgttttttcacgataatataaatttttacttgttccaaaagaacatatttttgcctggaagaagagtaaatcaatttttgtaaatgaatgccacacacacacacacacacacacgcaactaactttgtaatgaattgtttttaccagctctccttaatactctttttatctaaaaatcaatgtttttaggttttagcagtttgtgttataaaagacagaaatttcttttcctatagggtggataaggtaactttcatttttgtcttgctttccctgagtctgtcagtcagaagaaaatactgactttgaattagtgtcaagcttgtaactagttgtttttcactggactgttgatacattcttagagattttttcattgctgtttagattattttctcttcctttctacaactgttctctttcagctctcccttatatttttcttcttggaaaagtatctagttaattttgtattttaaagttcttaacctcatcccaagaacttctgcctttataatcatttctctcttcatatttattttatacaagatagatggttgattatattgttgctgtctttacagatggagaattaaggaataaaaaggacagacagactagattacctcaaacactgaaatgctatgatttgtcccaagccatacactcattatctgaccaggcatatggtgctttttctgtagaagcattttgtcattatgaaaaaaattatgtcttctgtatttaccttaatgaagctgacaactttcagaggttttcagtggtgaagagaaacttggataaggagataacttacacaaacagaaaaagcctcatctcttctggattcctttaatgccagtagtcatcaccaggtacaggttaaccacccttcaccagagctgggacctccaccttctcatctacaggtatatcaccttaaaaatcttctgcgtttcactgtcactcaacacattttctccttccaaggctctgcagtctcatgagtttgtctctatttttcttcttataacttaccttcctaccctagccagcctggatctcatagaagattaactgaaatactcccaacattgggtcattcctgtgcccctgttttcctgctgtaccctggtaaaatgccaacctaatccacagtctattgttctacttctgtatgtgagggctaaagaaaaatcaaccagtcatacagattaactctgttttaaagtaactattaatgctgggtgtgatggctcagacctgtaatcccagcactttgggagggcaaggcaggcagatcatgaggtcaggagtttgagaccagcctggccaacatggtgaaatcccgtctccacgaaaaatacaaaagttagctgggtatggtggctgatgcctgtaatcccagctactcaggaggctgaggcaggaaaaccacttgaacctgggagacagaggttgcagtgagccaagatcacaccactgcactccagcctgggtgacagagtgagattccatctcaaaataaataagtaaataaataaataaataaataaagtaactattaaatattttcaacctcatctgagtacttagtcttgcctgatgccttttgcttgcccaacactgttggcaagcaattctcattttcttcaagaattttaccaaatcttttccttctccctcttgacattcctgtcgcttcacctgatacagaggttcttaactcctacctcattgattttttttttagacagagtcttgctctgttgcccaggctggagtgcagtggcacaaccttggctcactgcaagctctgcctcctgggttcatgccactctcctgcctcagcctcccaagcagctgggactacaggcagccatcaccatgcctggctaattttttgtatttttagtacagacatggtctcaccgtgttagccaggattgtcttgatctcctgacctcgtaatctgcctgcctcagcctcccaaagtgcagggattacaggcgtgagccaccatgcctggccctcattgagatttttaaggtttttgcttttttctataaattgacctataggagtgagaggtgaagccaactggacttcctgggtcaagtggggacttggggaacttttctgtcttacaagaggattgtaaaatgcaccaatcaggaactttcctgtcttacaagaagattgtaaaatgcaccaatcagtgctctgtaaaacacaccaatcagcactctgtaaaatgcaccaatcagcactctgtaaaacccactaatcagcaggattctaaaagtagccaatcacggagaggatgagaaaagggcactttgataggacagaaatgaaacatgggaggggccaataagggaataaaagctggctgcccaagccagcagcagcaacctgcttgggtccccttccatgctgtggaagcttttcctttcactcttcacaataaaccctgctaccactcactccttgggtccatgccatctttaagaggtgtaacactcaccgtgaaggtctgtggctccattcttgaagtcagcgagaccatgaacccactggcaggaaccaactctggacacatcttgggggctcatccaggatatcaccacatggtgagtaccatcagaccccttttgcttgctattctgtcctatttttccttagaatttgggggctaaacaccaggcacctgtcagccagttaaaagcaaccagcacagccaccaggctaaagacacaggtgtcaggctttctgggaaaaggctctctaacaaacctgactcttcagagttgggagcattgatttgcctggaaccagcttccacttttcctgcacttccaggctgagccaagggtcaacagagaggaaagccattcagctctgaggtcctgacaaaaagttggttgacccttcagccatgagcagaactcatagttacatcggccgagtgagacttgccgatctatactatctatcctgacccttgcctcctgggtcctaatgcctgtcagacaaacttcctcccacctcttctctgaggctagtcctgattctaaaaacctctccttgtctctggtgctcttctagtatctcctataaggatgatttctaatataaactttgggactctgttcccttctttaggcacccaggctcaccaatcagaaagatataatttttgcccaaagccctgttaggggggtactagctggaattttaggatccctcctaagactagcaggtctaacaaaggctattcccaaagctatgatattgggagcctcagaaattacagactccaaaataggggggatatcctttctattaatatgatgagaagtgaggacaaaatgtgtcactcttccaaccctggagatcctttccctccctcagggtatggccttctactcaattttgaggcatatcatctttttaagacaagagtaaggtccccataccaacagggaaaaatgctaaggactttaactaacaggttttcaaaaatgcatcagtaagggccactaaatctgaccttccttggtcctctttgtgaactaagaggaaaggcagtgtttctgctgctgcttcagtgagtgtaactgttccaaacagcagggtccagaaaccattgtgggttcttgggtggggaaaaaaaaagaccaaaactgtgggtggctttttctttcatatgggaaatactcaagcatcaacaagctcacccttgacatgcatcctaagccattagacctatttgacctgcaaaccctgaaaaataagcagcttatttttttctgcactatggcctggccccagtattctctctctgatggggaaaactgaccacctgagggaagtataagttacaatgctatcctgcagcttgaccttttctgtaagaaggaaggcaaatggagtgaaataccttatgtccaatctttctttttattgaaggataatccacaactatgcaaagcttgcaatttatattccacaagaggccctttcagctcacctccatatcctagcctccctacagctccccttcctattaatgataagcctcctctaacctcccccacccagaaggaaacaagcaaagaaatctctaagggaccacaaaatgccctgggctattggttatgcccccttcaagctgtagagggagggcaatttggcccaacccaggtacatgaccccttatccctctctgttttaaagcagatcaaggtagacctgggaaagttttcagatgatcctgataggtatatagatgtcctacagggtctagggcaaaccttcaacctcacttggagagatgtcatgctattgttagatcgaaccctggccttgaatgaaaagaatgtggatttagctgcagcctgagagtttggagatacctggtatcttaatcaagtaaatgatagaatgacagccaaataaaatgacaaattccctactggttaacaagctgtccccagtatggatccccacagggacctagactcagaccatggggactggagttgcaaacatctgttgacctgtcttctagaaggactaagaagagttaagaaaaagcccatgaaatattgaatgacatccaccataactcagggaaaggaagaaaatcctactgccttcctcaaatggccatgggaggccttaagaaaatatattcccctgtcacctgactcccttgaggtttaattgatcctaaaagataagtttattacccaatcagccaaagatatcaggagaaagttccaaaagccagccctggaccctgaacaaaatttggaggcattattaaacctggcaacctcagtgttctataacagggaccaagaggaacaggccgaaaaggaaaagtgagataagagaaagaaaacaaacaaaccttggtggttcagagaggacagaaaatggaacagaccaatcacccagtagggcttgttatcagtgtggtttgcaaggacaccttaaaaaagattgtccagtgagaaacaagccaccccatcgcccatgtccactatgccaaggtaatcactggaaggtgcactgcacctgaggacaaagtttctctgggccagcagcccctaaccagatgatacaacaacagaactgagggtgcctggggcaagtgccagctcatgtcatcactctcactgagccccaggtaagtttaaccattgagggccagaaaattgacttcctcctggatactggtgtggctttctcagtgttaatctcctgcctcagatggctgtccccaatgtccattaccatccaaggaatcctggggcagcctgtaaccaggtatttctcccacctcctcagttgcaattgggagatttagctcttttcacatgcctttcttgttatgcctgaaagtcccacacccttattagggagtgacatattagtgaaagctggagctattatctatatgaatatggggaacaagttacctatttattgtcccctacttgaggagggaatcaccctgaagtatgggcattggaaggacaagtcagatgggcaaaaattgcccacccagtccaaatcaggccaaaacaccccaccacttttccttgtcaaaggcaatatcccttaaggcctggagcccataaaggcttacaggagattattagacatttaaagttcaaggcttagtaagaaaatgcagcagtccctgcaacaccctaattctaggaatacaaatactgaatggtcagtggagactagtgcaagatctcagactcatcaatgaggcagtaattcctctatatccagttgtaccaaacccctatatcctgctctttcaaataccagaggaagcagaatggttcaccattctggacctcaaggatgccttcttctgcattcccctgcactctgattcccagtttctctttgcctttggggatcccagaccacacgtcccaactcatggggatggtcctgccctaagggtttagggatagccctcatctgtttggtcaggcactggcccaagatctaggccacttcttaagtccaggcactctggtccttcagtatgtggataatttacttttggctaccagttcagaagcctcatgccagcaggctgctctagatctcttgaactttctagctaatcaagggtacaaggcatctaaactgaaggcccagctctgcctacaacaagtcaaatatgcaggccaaatcttagccagaggaaccagggccctcagcaaagaatgaatacagcctatactggcttgtcctcaccctaagacattaaaacagttgtggaggttccttgggatcactggcttttactgactgtggatccctagatataatgagatggccaggccactctatactctaatcaaggaggcccagagtgcaaatactcacctagcagaaagggaaccagaggcagaaacagccttcaaaaccttaaagcaggccctagtacaagctccagccttaagccttcccacaggacaaaacttctcttcatacatcacagagagagcaggaatagctcttggagtccttactcagactcatgagacaactccacaaccagtggcatacctaagtaaggaaattgatgcagtagcaaaaggctggcctcactgtttacgggtatgtaaatggcatactaggttccaaaggaaatttatggagatcagacaactgcctgcttagatagcaggtgctactccttgagggactggttcttcaaatactcatgtgtgcagccctcaaccctgctactttactcccagaggaaggggaaccaatcaagtatgactgcccacaaattgtagcccagacttatgccaccagagacgatctctaagaagtccccttagctaatcctgaccttaacctgtaccctgatggaagttcatttgtggagaatgggatgcaaagggcaggttatgccatagttagtgatgtaacagtacttgaaagtaagcctcttcccccagggaccagcactcagttagcagaattaatggcacttacccaggccttagaactgggaaaggaaaaagaataaatgtgtatacaaataggaattatgcttatctaatcctacatgcccatgctgcaatatggaaagaaagggagttcctaacctctgggggaagctctattaaataccacaaggaaatcatggagttattgcacacagtgcaaaaacccaaggaggtggcagtcttacccagccaaagccatcaaaaagataaaggagaaaaggcagaaggaaacagttgggcagatgctaaggccaaaattgctgccaggtggaacctcccattagaaatacctatggaaggacacttggtatggaacaatcctcttcaagagattaaggcccagcattccctggccaaaacagaatggggactttcacaagggcatagttttctctcctcagggtggttaatgatagaagtgggaaaggtactcatacctgaagacagccagtggaaaatacttgagaccctccaccaaacttttcatatgggtattgagaacactcattaaatggccaaatccctatttacagggccaaatctcctccagaccatctgacaagtagtcaaagcctgtgaggtgtgccaagggaataatcccttggtccatcataaggctcctctgggggaacaaagaatagggcactatcctggagaggactggcagttagacttcacccatatgcctaagtcaaggggatttcaatacttgttggtcggtgttgatacctttataaattggatagaagccttcccctgcaagacagagaaggctcaggaagtggttaaagtcctaattcacgaaataattcctagatttgggcttccccaaagctcacaaagtgacaatggtccagcttttaaagccacagtaactcagggaatttattggtcaagtccttcccctctacctccccttctatggattccttgtgggaaggaccatactcggtaatggtctctaccccactgtagttaaggtggcaggagtggaatctcagattcaccacacccaagttaaactttggactccccctgaagaaccttcaggaccatcagttcagatcccaagatcagccagaccagcctcgatacacctgtgaaccactggaggacttgcctctcctatttcagaaggaaacatcccagactaaaaaggctcctacacctgatcctgaggaaaaaccccttcctacttaaaagagataaatgaaaacctacataaatctttttttatcttttaatttttttattatactttaagttctagggtacctgtgcacaacgtgcaggtttcttatatatacatgtgccatgttggtgtgctgcacccattaactcatcatttacattaggtatatctcctaatgctatcccttcaccctccccacaccccacagcaggccccagtgtgtgatgttccgcttcctgtgtccaagtgttctcattgttccattcccacctatgagtgagaatatgcggtgtttggaaaaacctacataaatttttaacacctcaccttgtccctttaatggaatccttttactgtttcatcacattactaagtggcatactaaccatactctttgcagaaggactatatactgtagctcctgcagggacaaaaatcctaatcacatcaaccttttttctatcatccttccttctgacagaaatgtactcctacctttaacttagactagataaaatgatctcatcttccagggcatcctctttaccttcctatttactctttgcctatctatccctcctgcttccttaggtaccccccatagtcacctctccccttccactagctcttaattcctctaagagactctcaaattaaccctctctctgttaaaccagtccaatccttccctggcaaacgactgttggctttgtgtctctctatcagctactgcttacattgccactcccgttcctgcaaaaaactgggtctttattaatttcacctaccaccccccattatgaaggaaaagaccctttccaacttctaaatgtgcaattattagctgacttccccacctctgataggaccaaaaataccctaacaggacatgcaatccagcttttacgctcttacatttccaacctcacctattacatggacactgaaaagcccatacatggccctgtaaccaaaaataccatcttaactttccaagcccctttatgcatctaacgcaacctgttatcaggcctgtccctgggacacctactatctcatcagtgtaattacaccctacaacttcaagccccaactgatgatagtaacttctgagtcacccacacagctccattcagacagcttgtctgcttctcagtgaccccaaaatcatcacctcctccctgcttaataaacagtctgggttttttaatggcaagcatactccctgcatgaccattcacccctggaccccctgcagcagcgcccccaccactaataaatgccttctcatcccctctttcaattactctcttgaatggttcctagtagatacaaaaaggttttttctccaatgggaaaatagaacacagggaaccactctgtttgctccctttccagccgctcaccagagctaccttggcaagtagtctagcagaatgggaaaatgaaaacaacaaactcacacacctttttaacacacacaaccagttctgtctacccagccaaggcatattcttcttatgtgggacttcaacctatatctgcctccccaccaactggacaggcacctgcaccttagtcttctgaaatcccaatattgacattgccccaggaaatcagaccctatcagtgcccctcaaagctcaagtctgtcagcacagggccatacaactaatacgtgtacttacagggttaggaatggccactgctacaagaaccagaatagccagttcatctactttattatcctactaccacacactcaaaggatttctcagacagtttgcaagaaataacaaaatctatccttactctacaatcccaaatagattctttggcagcagtggctctccaaaaccactgaggcctagacctcatcactgctgagaaaggaggactgtgcaccttcttaggggaagagtgctgtttttacactaactagtcagggctagtataagatgccacctggcatttacaggaaaagtcttctgaaatcagacaacacctttcaaactcttctactaacctccggagttgggcgacatggcttctcccttttctaggtcccatgacagccatcttgctgttactcatcttggggccctgtatttttaacctccttgtcaaatttgtttcctccaggaatcgaggccatcaagctacagatggtcttacaaatggaaccccaaatgagctcagctcacatcttctgccaaggacccctggaccgacccactggccttttgactggcctagagagttcccctctggagggcactacaactgcagggccccttctttacccctatccagcaagaagtaggtagagtggtcattgcccagttcccaacagcagttgggatgtcctgtttagaagagggattgagaggtgaagccaactagactttctgggtccagtggggggacttggagaactttttggtcttacaagaggattggaaaacacaccaatcagggacttccctgtcttacaagaggattgtaaaatgcaccaatcagtgctctgtaaaatgcatcaattagtgctctgtaaaacccacaactcagcaggattctaaaagtagccaatcgcagggaggatttaaaaaaggacactctgataggacagaaacagaacatgagagggaccaacaagggaataaaggcttgtcaccccagccagcagcagcaacccactcaggtccccttccacactgtggaagctttgtcctttcgctcttcacaataaaccctgctactgctcactctttgggtccgtgccatctttaagagctgtaacactcaccgcaaacgtctgcagctccattcttgaagtcagcgagacaacaaacccaccagcaggaaccaactccggacacaggagtatccttacccctttctgatagattttaggggatggagtatttgtcctgagaaggccaatacatccatctgacacttgtctctatccccatttcctatgtaagtatttgactcttcagccatccagtacactggctctgtctttctggtttacacccgctatccctgtgtcagcactcatttctgctttagcccattaacttcctttctcacaactctactgaacttgtcaataggcacatcagtgatctcctgcatgcccaggtctcgtggacatttttcttcatgttttttgactgctgctctgcctcacttgatattacaatctcctttccttgaaaccctaggctacggtagcactacactttactggttttccattacctttcttacttctcctttgttttttttccagagtccctccagatgtggcctaggaatccttttaaacacagtgctccaggcagaaattagttggagttagtttacaagatgcaatttatttgccatccatccttgcatttcagttccattatttgtcctcaacatgactgtctataaaaacactttctccttccctgggatggcagctaacaagacctagcaaagagaaatgggaaattttattcacaatttcacatgggaagcaattggtaagattattttactgccaaggataataaaattaatgagagagttgaagaaagaggcatgaattctgaaattatatagaaccatctaagaggtaaaactgaggtgttttatccataactgtaggctgtgtttgttttcatagaagtactgtctgatttatggacatgccattgattacactagaattacagattgggcacattaagcacaccaacttaaaaatccttgacattttaaaaatacctctggcagggtttcagagtactcatgatatttaaaatagtaccatgatttgttggtttctgaagtataattttgtttagtcacatgtatgataagaacatacagacttcttgctgtgggcgttggctcatgcttataatgcctgtaatccccgctactcaggaggctgagtcaggatgattgcttgaggccaggactttgagaccagcctggacaatatagcaagactgtctctaaaaaaaataacaatcataaaaagattccttggtttgtaggattaatttttttttcatttttctcgcattaatctgccacttttatctaagataccttaagtttctttattccttggattcgtagtgcagtgaaatgattagggcagggttttgaaaccagatacctgggttcaaatactggtgtggactgtatatttgggggcaatggccttaaatcctctgactttccagtgtcttttaaatgagtaaaataagccttactttgtggaatgttgcaaagatgagagaagatgtatgtaaaatgtttacctattacagtgcttgatatgtagtagacaaattctaaaattacactgaaccattttgcccattgtggctttagaatagaaaagcatactcatacattaaattgtgcacctagggccaggagcggtggctcacacctgtaatcccagcactttgggaggccgaggtaggcagatcacgaggtcaagatatcgaggccatcctgaccaacgtaatgaaaccctgtctctactaaaaatacgacaattagccaggcgtggttgtacgtgcctgtagtcccagctactagggaggctgaggcaggggaatcacctgaacttgggaggcggaggttgcagtgagccgaaatggtgccactgcactccagcgtgggcaaccgagtgagactccgtcaaaaaaaaaaaatgtgcacctatagctaagtgactccagccctgtggaacccttcaatgccaaaatggcatgtaggactagatagatgaatgtcacctgaccctgatcgtctattagtactcactgtattttattactattatttggagacagagtctcactctgtcacccaggctgaagtgcagtggcgcaatctcggctcactcactgcaacctctacctcccaggttcaagtgatttttctgcctcagcctcccgagaagctgggattacaggctactgccaccacacccagctaaattttgtatttttagtagagactgggtttcagcatgttggccaggcttgtcttgaattcctgacctcaggtgatccgcctgcctcggccttccaaagtgctgggattacaggcatcagccactgcacccagccaactcgatgtattttaaaatcactcttactggtcattagtcttattacatatcagtataatgaacattttatttattcaagtcctttaattttttttaggtggggataataaaatttcagttgcattccacctgtttcttcatgtgcatatctggatgcacatgtaatttgttggtaccagttttcattgctttactgagcatttgttgagtatctatgatgtggaaagtattgtgccgggtgctggagacagagaagttgagtaaagcatagtgtcatcatccctgtcttcagtgagctcagccaggtggggtggggagcagaaaagaaatgtgaatcgatagttccagggcaaagtaataagtgctttattatgatatgcaaaggtttgtgcgtaccctagaggagcacatgaaagggagtcattaacttgctgaagcttctgaaagggctttagagaagtcgaacatctgatctggttcttgagtaatgagtaggtgttcaccaagtagaggttaaagaaagattgaaaagttgaaactcaaacctcatgaccatgcctccctgtcttctcttctcaaatccatatcattcctgatacattattaaatggatctattttgaaacgtcatgcatttactcccagtccaaaccctctcccaggttataggcctcactcatgcatctgaagaggacacacagggcaaaagaaagagagcagtgggtgggggggctctgagggtcacattggtcaaccaagggtgccattctttttccttacaaatcagatttactgaggtagaatgtacttacagtaaaaatgtatccattttaagtttatagtttggtacattttaacaaacgtatagactctttgccaccacagtgaagatagagaacatttctatcaccccaaaaaaattccttatgccccttttgagtcagtccccatcccccagtcccgggcaggtgctcactggctttctatcactatagattagttttaccttttccagaattttatacaaatgaattcatacagtttgcacttatttcacctagcatagtgtttctaagactcattctttttcttaaatgtcagtcattgatttctttctacagtggaataatttctattgtatgatatatcacaatttccttatccattcaccattgggttgttttctggttttggctattacaaataaaaccattatgaacattcatacacaaaactttgtgtggccatatgttgtcattcctcttgagtaaatactaggggttgtattgttgagttgtatggtaagtatgtgtttcactttataagaaagtgtcaaacttttccaaagtggtttatcatttttgcattcctgtcagtcgtgtgtgagagttcaaattgctttaagtccttgccaacacacccttctattgtagccattttagtagaagtgtaatggtttttattgtggttttgctttgtatttctctaaggactggtgatgttaagccttccttcaagtgtttattagccattcatgtatcttcttttttactattcacaactcacagattgttgtgacatttttgtatcttctttagtaagtggtctgttcaagtcttttgcccatttttatggggctgtttctattgggttataagggttctttctgtactctggacataaggcgtttgtcatataaaggttttgcaagtattttctttgagcttgtgacttgcttttcattgccatatggtgactttcaaaagaacagaatttttcattttgatgaagtctagttgatcagtgttttcttttatgtttcatttatgtttggcctatttaagaaatcttggctatcctaaggtcacaaatattttgtcttatgttttctctgagaagttttagttttagcttgttttttgtttgtttgttgatttttaaacccacagccaccatcatacagttttagctttaacgttttggcttatgatccactttggcttcacttttgtttttggtattagttaaggatcagggttcattctttttttttttttccacatgaatatactgttatttcagtaccatttgttgaaaagactctcctttcctccattgaatcatgttgacatctttgttgagaattaatggaacacatgttagtttatttctgaactttgttctgttccattgacctataggtctatccttacaccagttgcaccctgtttggattactgtagctttatagtaagtttcgaaatgaggtagtgtgtaagtcttccaaatttgttctgtaccttcaaagttattttgactcctctgggtcctttgcattttcatataaattttaatatccgattgtttagaagcctgttgaaattttaaataagattacatttaatctagagatccatttggggagacttgccatcttaataacagcaaaccttccaattcagaaacagcatattttaaaaatttgtttaggtctcctttaatttctctcagtaggagtttatagttttcagtgtaaaggtcttatacaaattatgttacatttatctttaaatattttctatttgggaatgtgattataaatggaatgttaagagttttaattctccaactgtttttgtaagtatagaaatacagttgattgctgtgagaggtgaagctggctgggcttctcagtcaggtggggacttggagaacttttctgtctagctaaaggattgtaaacgcaccaatcagcgctctgtgtctagctaaaggtttgtaaacacaccaatcagcactctgtaaaaatgcaccaatcagcgctctgtgtctagctaaaggtttgtaaaccaatcagcactctgtaaaaatggaccaatcagtactctgtaaaacagaccaatcagcactctgtaaaatggaccaatcagtaggacatgaccaatcagtaggacgtgggcagggccaaataagggaataaaagctggccacccgaaccagcaggggcaacctgcttgggtcctattccaggctgtggaagctttgttgtttagttcttcacaacaaatcttgctgctgctatctctttgggtccgcactacctttatgagctgtaacactcactgcgaaggtctacagcttcacttctgaaggcagcaagaccacgaagccaccagaaggaaaaaactcccgacacatcttaacatctgaaggaacaaactccagacacaccgtctttaagaactgtaacactcaccacgagggtccgcagcttcattcttgaagtcaacaacaccaagaattcaccagaaggaaccaattccggacacacctgtgtgtaattttttatcatgagaccttgccaaatttacttagcattaggttggtgcaaaagtaattgcgtttttttgctttttttttttttttttttttttttttaagcaatggcaaaaaccataattattttcacaccaacctaatagttttaatagcttcttggtaaatcccttaggattttctatgtatccaatcatgttatttaaaaacgcacttttatttcttttctttccaatctgtttccctttatcctctccctcactcctcctcccttattgcactggccatggcctctaacacaatactgaacaaaagtggtgaatgcaaaacattcttgccttgttttgtgtcttagcaggaaagcatttaatattttaccaataaacttgatattaactgtaagatttttgtacatggtcttaatcagtttgaggaagaacttttccctttgttttgttttgtttttgctgaaacctgtttattgttgattgttttatcttttaatttcaaattttaggcacaataattgtaaaaaaaaaaaaaacagatttttcacatatgcctcatccacctttcccaatgtcagtgtcttacctaacgctagtacaaatatcaattagcaaattaacatgctgtgtacaaggtattggtccagatgttaaatgtatgtatgaaatctgattttgaagactaaagtggatattaatataaacaattattttttgctttgcaaaataaattccagatgaaattccttttaatcaaatttttatgttgtatgtaatcaaagtatttggcaagcatctcttgttgagaaacaagattcagagagagtagaggtaggccaaggtctcataactatttagtagtagagccctcactaaatgcatatttcctaaccaattcccacatcaccagccttctctgtgattttcttctttaaatattgattggcattgtattttgctttcattgaatttgtacttcattatatttgtgttttattttcattgtataaccaccatggcataatgttattgagaactgaaagatcagttactctgtctttttaaaaaatgtttaaaatgaaataataatctgtacatggttgtggtcccagctacttgggaggctgaggtggaaacatcttttgagcccaggagttgaaggttgcagtgagctatgaccatgccaccgcactccagcctggggtgacagaaggtgatcctgtttctaaaaaaaataaaaataaggccgggcatggtggctcatgcctgtaatcccagcactttgacaggccaaggtgggtggaccatttgaggtgggaagtttgagaccagcctggccaacaaggtgaaaccccatctctactaaaaaatacaaaaattggcaaggtgcagtggctcatgcctgtaatcccagcactttgggaggctgaggtgggcagatcatctaaggtcggaagtttagaccagcctgaccaacatggagagaccctgtctctactaaaaatacaaaattagccagttgtagtggtgcatacctgtaatcccagctactcagaaagctcaagaggcaggagaatcacttgaacctgggaggcagaggttgtggtgagccaagatcatgccattgcactccagcctgggcaacaagtgtgaaactctatctcaaaagaaaaaaaaaattaggtgggcatggtggtgcgcacctgtaattccagctactcaagagacagaggtagaagaatcacctgaacccaggagaggaaggttgcagtgagctgagatcgcaccactgcactctagcctgggcaacagagcaagacactgttcaaaataattaaatatatatatatatatatatatatatatatatatatatatatatataaactgggataataggctgggcacagtggctcacgcctgtaatctcaacattttgggaggccgaggtgggtggatcacctgagaacgggaattcaagaccagcctggccaagatgggaaaccccatctctactaaaaaaaaaaaaattagccaagccccatgctcagtgcctgtaatcccagctactagggaagctgaggcaggagaattgcttgaacccgggaggtggacattgcagtgagccaattgaccaggctcgtctcgaacttctgacctcaggtgatccacccaccttggcctcccaaagtgctggcattacaggcatgagcccagcctattatttatttttaattatacattaagttctgggatacatgtgcagaacgtgcatggttgttacataggtataactgtgccatggtggtttgctgcacctagcaacctgtcatctaggttttaagccctgcatgcattaggtttttgtcctaatgctatccctccccttgcccacaaccccccaacaggccctggtgtgtgatgtttccctccctgtgtccatgtgttctcattgttcacctcccacttatgagtgagaacatgtggtgtttggatttctattcctgtgttagtttgctgagaatgatggtttccagcttcatccacgtccctgaaaagaacatgaactcattcttttttatgtctgcattgtattccatggtgtatatgtgccacgttttctttatccagtctatcactgatgggcatttgagttcgttccaagtctttgctgttgtaaatagtgctgcaataaacatacatgtgcatgtgtctttacagtagaatgatttataattctgtgggtatatacccagtaatgggattgctgggtcaaatggtatttctgattttagatccttgaggaatcaccacactgtcttccacaatggttgaactaatttacactcccaccaacagtataaaagcattcctatttctccacatcctctccagcatctgttgtttcctttttaatgattgccattctaactggcatgagatggaatctcattgtggttttgatttgcattcctctaatgaccagttatgatgagctttttttcatgtttgtttgccacataaatgtcttcttttgagaagtgtctgttcatatcctttgcccaatttttgatggtttttttttcttgtaaatttgtttaagttccttgtagattctggatattagacctttgtcagatggataaattgcaaaaattttctcccattctgtaggttgcctgttcaccctgatgatagtttctttggctgtgcagaagcactttagtttgtttgttttaatatttgtgtaataatgtccagttttaccaagcagaatgttttacataggagcttttatgcaaaactcattgttcttcacttagagcttgttagtccagagtgaaatcaaatccattttcagttgctacgtggctttgttaaacagaaacttgagttgaggatgctctgctatatggaagaattttcactcccctgatgtgtttgtataatagaatgtatttttcccctttcccctgtgaaaactttatggaaagattatttcagtgttaaccctgactgatattttactaatcaaacaagtgtctgttactcctttattcattaatttatcaagcacttaatgagtgtcttctgtgtgctaggcactgcactagactctggggagtcaaagatgaatcacacttagtctctaaccactgggaattcattctggttagagaaattataaacaaataattatgatcctatgggatatttttaaagtatgaaatgagataatgtatgtgaaaaaaactttgtaagccttacacatttaagattcttttctccagtggatagcatgatgcctgacatatttttaaatgctcattatacctttaaatgaactggtaaagagccaagagttatgagagcataatactagagtgtagtagatggatttgttctgccttggtggacaggtgggatgtggagatttccaagaatataacttttgaactggacttggaaatataatttgtttaagaatcgagaaaggaggttctgcgggggcagctatcagtcgttaaactgcctggatttagatgaaccacgtatctataggcttttacagtttacaggatgcttttgtttacagtatctcttttgagccttatgacaggtccatgaggtgagttcatacaaccaagagaagccttgaccagaacctgggttctcaggtgtaagagacagcatctggccagtttattctttacagtttcatggatgttatgattgctaatattattgtatttctattattatatttgtgaaagcatggttttttgagttacaagtatgtgaaagaaaaatgtatacatgtcatgaaatgtatagttttctttttatttctctaaagaagtttagcttgtattaatacatgaggagtttttttccagaggtttcagaaacaagttagaaaatgcttccataatatttcatagattatttaagattcaagcagtttttcaggatttaaaccatttaaaccatttatctaatgaacagtaagtagtaactctgagctctacattctcttgtaagctttctttttctgttatagttgagcacatttgttgacatttttattttttacaccaaaatgtttggttaaaatactttggggagaaaaataaagaacttttttctcatttatagttgacttcctattttgtgtatattgtcttctgtatgtattaagacagtgaagtattggtgaaagaccaggtaaatagatcaatggaacagaataggcaccccagaaatagacctatacaaatagagtcagctgatctttggcagaggagcaaaggcaatttaatggagaaaaaattgtcttttcaacaaatggtgctggaacaactagacaaccacatgtggaataatgaatatagacactgaccttacacctttcccaataattaactcaaaacaggtcatagacttaaatgaaaaacaaaaaactagaaaatttctaggagtttaataacataggggaaaatctaggtgaccttgggtttggcaatgagattttagatacaacacaaaaacaccatctacaaaagagaaaatggacaagattgacttcattaaagtaaaacatgtctgctgtgccagtgacactgttaagaatgaacagacaggtgggcacggtggctcacacctgtaatcccaacactttgggaggccaaggtgggcagatcacaaggtcaggagttcaagaccagcctggccaacatggtgaaactcatctctactaaaaatacataaattagctgggcatggtggccagcacctgtaatcccagctacgcaggaggctgaggcaggagaaatgtttgaaccctggaggtggaggtttcagtgagccaagattacaccactatacaccagcctgggtgacagggtgagactccatctcaagaaaacaaaacaaaacaaaacaaacaaacaaaaaaactaaatatggtcttaccatacattctagcagtcatgctcctagtgattttcctaaatgagttgaaaacttatgtctacacaaaaacctgtatattaacactttacagaagcttcattcataattgccaaaagttggaagcaatcaagatgtccttaaataagtgaatggataaacaaatggtggtacatccatgcagtggaatattattcagccatactaagaaataagctatcaagcccagaaaagatgtggggacactttaaatgcataaaagcagccagtctatgaaggctacatgttgtataattttaactatatgacattctggaaaagaaaaaactatagagacagaaaaaagatcagcaattcttgggttgaggatgggaaagggaggaaaaacgaataggtgaatcacaggctacttagggcagtaaaactcttctgtatgatactgcaaggatggatccatgacaaaacccccaccatcgtgcaatgcaaagagtcaactctaaagcatggcatttagttagtaatttatgggtactgggtgatcagttgtaacacatgtgccacactaaggcaagaggtaatcacaggtgaaggagaaggggtatacagtgtgtaccttctactgaatatttctatgtgactaaaactgttccaaaaataattatttttgtttttaaaaaagtaaaaagtgattttccagtatctcactgctaatggcttagcctttttttttattctttccttgaactgagtaggaatctgagtctgtcagttgcttcactttctgtctcccagtaacttgctgtgattatgaaaatgtttgtgctaaacaacccatgaacaaagcaaataaagatattccttcatctacccagactgaaaggaaatatgccaccacatcaatgttaatggaagagagattacagctggtttctcctttcatctttgttttcctgtattttccaaatgttctctgtaaaataaaaagctcttagcactaaaacagtatatatgtattccactgcagcttaattgtattcagttaacataattgaagagattaaataagaaatgtagaaataagtaagacatagatcttaaggatctctctggttaactgtaagtgaaagactatctcaccacaatgcttgatgtaaacggtctgattggtgagtgacaacatcatcaattattgcagcttgcaaagggattgatttttaaatttttacttattaattatatacttgcttatttgttttctcatttacttgctttcttatttgcttattatttactcactttcctattgctttaaaagaggattctaattagatattatctaagtgattaggccgagttcttacattttgttggtttaaattactttaagatatatgtttgaaatcacatgtttactcttcaaatgtgtgccatttgcagtgtgtgtgttgggggaaatgagttattttttgatgggaaatgagttattttttaaaaagagaattatttagatatggctttagtgattccttagcacgataactggtgatgaaatgtagcttattgttttcagctagtttacattggtcttgagacaaacagatatttgtttcttaattacaggctctagatgactttatagaacacctcagggctgttatcaagtacttacatacgaaggataactccatttgattatatattttataccccaaaggaaaataagtttaaatttaattttaaccagatgatgccatctgtatggagtcactctgtcattgtgtccatactggccaggatgcattttaatgaatagtgtttgtttatttggtcttagccaaatgagcaaggacctggagactgggaaatttgagaagacatttaggaatcactggcttttctctgttgatgccactatgtaaactaacgatgacggggtagagaagatgtgtggaattcctgtgtgattcactgtaactgtagtgtgctaaatgcatggttaagctagtgtcagcatcgtcttcctataaattacagatcccttctgtgagcagaactgcagatgttgtattttgaaatctgctttagaggatattttaagacttttaataaaagaaagatgactaatgtacaaggggactcaaggggaagaaaaaccccttcctctatagggtagcatatttgggattataatttttgttttctttagcttggtttcattttcaagtgaatccagaaaacactatttactttgacaatattgctataaaattaccataatatgaggatacgttgtcaatctcaaatatataaaatgaccaatgtcagcgagcatttatccttttaagaaaatctattctatgaaaatccaattttgtgcaaaaagtgggaaataaatggaggcataactgtttgctctacaagacttgatttgattcatgtaaatagaaaatggttttattacatgtaaaatatgatttaaattggtcaaaatgtcattttcagaaaagcataccttttaagccagcactcattacatagaacaaggattctatctgtgtgccacaaacaacttcaaggaattttctttgtatgtaatgacataggaagtttttccttagcgttcatctcaactacggttcctcaggaatattgtctttgagtacaaattataatgggtttattttctaactaaagtagagtagtcaggctagtttattagctagtttagttctgacctctatggttgagaacagatacaacagaaatgctattatatcttcagatcttatagaaaacagatagcggaaatgatgaagtgtacctggctggctttctgacaatttgtgtgaaatgcttggctttgtaattttacatttctttgtagtctgatgccttgagtttatttatgtagaaacgtatttccacagactgcttatctgatttttacatgctgcttcacagttgtatactacttaaggtatgtagctttatacttaatctaaattggaattgggtttttgtgttattgtcatttggtacattggtcaacactttataaatgtttaactttttgttttgagttaattatagatttgcatgtagttgtaagaaataatagagagacgccaggtgtggtagctcatgcctgtgattccagttacctgggaagatgaggcaggaggatcccttgaagcccaggagttcaaagatgcagtgagctgtgatggtcccactgcactccagcctgggtgacagtgagaccccatctgtacaaagaaaaacaaataatacaaaaaaatctcatgtacgggtctactaaattttattgtgttttgctttattgcgctttgcagatattattttttacaaactgaaggttggtggcaacgccgtgtcaagcaagtcttgcagtgctgtttttctaacagcatatgctcacttcgtgtctctgtgctacatttcggtaacagtgttttagatgttttcattattatcatatctgttatatagcgatcagtgattgttgatgttactattgtaattgtttacaacctcgctgatagaaaatttttgtgtatgtgtctttttgttttgtattcttttgttttgttttgttttgttttgttttgttttgtttttgagacagggtctttctctgtcacccaggctggagtgcagtagcataatctcagctcactgcaaccaccaccttcccggctcaagcgatcctcccacctcagcctcctgaataactgggacagcaagtgcatgccaccacactcagctaatttttaaattttttgtagagaagaggtctcattatatgcccaagctagtctccaactcctggactcaagcaatcctcccacctcggcctcccaaaagtgttgggattacaggcatgagccaccgcaccttcccagttgtgtgtgttctcactgtgtcattcaccatccattctcagctcgctcattctcctcaggcctctctattacctgacacagaacagtattgaaattaggctaattaataaccttgcagtagcctgtgagtgttcaagtgaaaggaataattgcatgattctcactttcgatcaaaaggcagaaacgactaccttcagtgaagaaagcataccgaaagccgacctaagctgaaagctaggcctcttgtgcccgttagccaagctgtgaaaacaaagggaaagttcttgaaggaaattcaaagtgccacttcagtgaatacacaaatgatgaaaaagcaaaacagacttattgctgatacagagaaagttttagcagtctggatagaagatcaaaccacccacaacattcccttaaggcaaagcctaatccagggcaaggccctagctcgcttcagtgctatgaaagctgagagaggtgaggaagccactgaagaaaagtttgaagctaacagaggttggttcatggggtttaaggaaagaagtcatttctataatataaaattgtgaggttggctgggcgtggtggctcacgtctgcaattacagcactttgggaggctgaggtggggggatcatgaggtcaggagttcaagaccagcctggccaacatggtgaaactccgtctctactaaaaatacaaaaattagaccaggcaaagtggctcatgcctgtaatctcagcactttgggaggctgaggcgggtggatcacctgaggtcaggagtttaagaccagcctgggcaacacggtgaaacaccatctctactaaaaatacaaaaattagctgggtgcagtggcatgtgcctgtaatcccagctacttgggaggctgaggcagaagaatcgtttgagcctgggaggcagagtttgcagtgagccaagattgtgccattgcattccagccttggtgacagagcgagactctgcctcaaaataaataaataaatgaataaataaaaagtgcaagtttgaagcagcaagtgctgatgtagaagctgcagcaagttattcagaagatctagctaagatcattgataaaggcaggtccactaagcaacagatgttcaatgtagaagaaacagccttttgatggaagaagatgccatttaggactttcctcctagagagcagaagtcagtgcctggcttcaaagcttcaaaggacagctgactgtcttcttaggggctaggatagctggtgactttaaattgaagccattgtttgtttaccatcccagaaatgctagggcccttaagaattatgttcaatctactctgcctgtgctctgtaaatttaacaacaaagcctggaagatatctgtttacagtataatttactgaatattttaagtccactgtggaaacctactgctcagaaaaaataattatttcaaaatattacttctttttgacaatgcacctggttcctcaagagctctgatggagatgtacaaggagaggaacattgttttcacgcctgccaacacaacatgcattctgaagcccatggttcaaggagggattttgactttcaagacttattagttaagaaatacattttgtaaggctaaagctgccatagatagtgattcttgtgatggatctgtgcaaagtaaattgagagccttgtggaaaggagtcaccattctagatgccattaagaacattcatgagcaggtcagaatagcaacattaacaggagtttggaagaattgattccaaccacccaggatgactttaaggggttcaagccttcaatggagaaagtaacagcagatgtggcagaactcgcaagagaactagaattagaagtggagcctgaagatgtgaccaaattgcagcaatccatgataaaatgtgaacaggtgaggagttgattttaatagataagcaaaggaagtggtttcttgagatggattctactggtgaagacgctgtaaacattgttgaaatgacaacaaaggattcagaattgtgtagaaacttagttgataaagcagcagaggggtttgagaagattgaccccaattctgaaagaagctctactgggggcaaaatgctaccaaacagcatcacttgttatagagaaatctcttctctaggccccagttttttctaagtgaagagaattttagaatcaatctcatagaagtgttgggaggatgaagttaaaacaagcaacacatttaggataggatgtggcttataacaagctctccatgaatattagctatcatcattcttaagaaaattaccaaattaatagattttctgctccttcacacttaatttagtagtttttttcatttattttgtcttcattaaagcagttggcacaaaattagtttgttgatctgcaatagattttttttttttttttttgagatggagtgtctctctgttgcccaggctggagtgcagtggcaccatctcacctcactgcaacctccacctcccgagttcaagcaattctcctgcctcagcctccccagtagctaggactacaggcacccaataccatgcccagctaatttttgtatttttagtagagacagggtttcaccatgttggccaggctggtctcgaacacttgatctcatgtgatccacctgccttggcctcccaaagtgctggcattacagggcatgagccaccacactgggccaattgacatgtatttaatagttattctcctagcaatcattattttgtattttctgtcttagcataaacaagtttcagtcttactgatgtgtgttaagctgttcccccatttatacttcagctggtttgtggtttttaataatgtagatacaaatgtgaggaatatttatatgtacataacttttcacttttaaaaagttgtgataggctgggtgcggtggctcatgcttgtaatcctagcactatgggaggccgaggcgggcggattgcctgagttaaggagttcgagaccagcctgtgcaacatggtgaaaccccttctctattaaaatacaaaaaaaattagcgaggtgtgggggcatgcacatgtagttccagctgatcaggaggctgaggtaaaagaatagcttgaatctgggaggcagaggttgcagtaaaccaggatcgcgccactgcaccccagcctgggagacagagcgagactccatctctaaaaaaaaaaaaaaaaaattgtgataaggtctggcacagtggctcatgcactttggcaggctgaggcagccggatcacctgagttcaggagtttgaaaccagcctagccaacatggtgaaaccctgtctctactaagaaaaatataaaacttagccaggcctggtggcaggtgcctgtaatcccacctactcaggaggctgaggcaccagaattgctcgaaccctggaggcagacattgcagtgagtcaagactgtgctgctgcactccagcctgggcaacatagcaagactgtctcaaaaaaaaaaaaagttgtgatataaaatatacattaacataaaatttatctttataaccattttgaagtgtacaattcagcggcaccaagtacattgacaatgttgtgtactcaccaccactatttatttgaaaatcttttcttcgccccacacaaaaacaaactatagccattaaacagtaactcttcacttcccctctccctagctgctactaacctctattttactttgtccctaagaatttgcccattctagatacctcatgtaagtgggatcatacaacattcgtccttttgtggctgtctttatttcacttagcataatgttttcaaggtttcatccatgttaaagcatggatcagagcttgagacagagtctcgctctgtcacccatgctggagtgtgatggcgcaatctcggctcactgcaacctccgcctcccgggttcaagtgattctcctgcctcagcctcctgagtagctggaattacaggcatgtgccgctgcgcctagctaatttttgtatttttagtagagatgggccaggctggtcttgaactcctgacctcagctgatccacatgcctcggtctcccgaagtgatgggattacaggtgtgagccactgctcccagcctattttatgtattttgtacatactgtttagccattgaacttttgatggccagtgagtatgactggggcccatccaggcagggtgtggagcagagggccctcctttatgggctggactcactagtgggctgcttacctgtgactcacctccaaggctcaagttaatgctaacgtgttgatttgctagaactctgagctgtgcctgtgtctccttgggccctgggagatttttttcttttagcagtattttatggagatattgtctaggtagagtaaaatgtacagatcctggttttctgttgagttttgttttgttatgaggcagggtcttgctctgtcacccaacctggagtgcactggtacaatcatagctcactatggcctcaacctcccaggctgaagcaatcttcctgcctcagtctccctgtagctgggactacaggcatgcaccaccatgccccactaatttttttgatttttagtagggatgatgtctcattatgctgcccaggctggtctcgaactcctgggctcaagcaattctcctgcctcagtctcccaaagtgctcaattacaggtgtcagctgctgcacccagcctcagcctgttgttaatggatgtaaacacctgtgtgaccaccacccagaaccagatccagaacatttccaggctctcgaagactccttcgtgcccgtcccagtcagagacccccaagggtaatgccattctggcctgttgttcaacttgacttgaatggaattttcacagcatgtgtctgtttggatctggcttcttctgctcagttgcgaggcccattgatgtttcctgtggcctagctcctttttattgctgagggccactcagttgtatgaaaaccacaattcattctcccttctcctttggtgaacatttgcactgtttccagcctgggacaatgatgagtaaagctgttaaaaacaggcacatatgttttcctttctcttgggtaattttttagaagtagaaaagccagccaatagaagtagatatttgactgtattaaaactcaccaagcagtttttcttattttttttaatttttaaattttttttagccagagtcttgctctattgcccaggctggagtgcagtgtcgtgcactctcatggacacacatggatccagatgccacagcagccttagctggccaaccttaaaacaacacatttgttcttatagtgttggaggtcagaaggctaaaatcaaagtgtcatggaaggtggccagcaggaccgtggagcctctgaggcagtgggacactttggctaagagccacatactggctctggctggcaggcagtcttggccagccaggctgagctccccactgtcggggcaagcctgggtgtcctctggtgatgctgagtccagaacaaggctgcccacattctggtccgcagggctcagctctgctttctagacttccaaataggaagaagagggaaagttcccagtctctgggcacaaccaagaacagagagcaagttctgactcagcagagggattgattcattccatgcaaagagctgcactgccaaggctgacacgagccagggactcagcacacggcatcgcacccagtcctctccagaactctgccacgtagtgttaccatcctgccgttcagaggagggaagtggggcacaggcaggtcaagcaacttggccaatgtctgaattgctagtgtacggagctgggaggggagcccatggggccataaatccagtgcaaaaatccgtcctcctgaaccctacggagcaaaagtgagtccagttgttttcctgggtttcctggacacacagatctgaatttcacagcagccttggctggccaatcttaaaacaacaaatttatactcttaatgattctggaggtcagatgttaaaaggtcaatgtgtcagcaagtttgtttccttctggaggctccaggggaaagaatctgtttcctttccttttgcagcttctagaagctacatgcatgccttggcttatgggccctcctgccatctttaaaactgtcagctttgtccaggcacattgactcatgcctgtaatccaagcactttgggaagccaagacaggaggatctctttagtccaggagttggagaccagtctgggcaagaaagtgagaccgtgtctctacaaaaaaatagaaaaaaattagacgagcatagtggtgcatgcctgtagtcccagctacttgggaggctgaggtggggggaatcacgtgagcctggggaagtggaggctgcagagagctgagatcacaccactgcactccagcttgggtgacagagtgagatcctgtctcaaaaaaaaaaaaaaaaaaacacaaaacaaacaaacaaaaaaccaaaaccagcttcttcttgcttctgttgtcacatctgcaggactcattctcccgcctcccttttataggatccttgtgatcacattgggaccacctgtataatccaggacaatctccccatgccaaaattattgatttaatcacatctacaaagtcccttctacaacgtattcacagtttctggggatttgaatgtggatgtttctggggggtggcccactgataaccagtgttataagtttcttgaaaatccttccagagatattttatctataaataaataaatgcaagtccatagcctccccccacccactttttatacaagtggcatcattccatatcagtgctcagttccatcttcctctttttcttttctttttaaggttacacagaacctattatatggaaataccctaatttatttcacttttttcctgttggaagacttttaagctctttccaatgctttgctttataatcttttgtattataatgctgcaacctatataatcctataaccttgtttgtaggataactttctatttaaaatctcatttttcaggccaggcatggtggttcatacctgaaatcctagccctttgggaggctgagacagaaggatcacttcagcccaggtgttcatgaccagctctggcaacatatcaagaacctgtcttttcaaaaaacttaaaaattagtgaggcatggtggcatgtacctatttggtcccagctattcgggaggctgacgtgggaggctcacttgagcccaggaggtggtggctgcaatgaaccaggatcacacccctcactgcactgcagcctgggcaacagagtgagaaccaatctcaaactaaaataaaataatcttgtttttcagaatttgagggtgccatttaagacattttgacacttcacagaacttttttggggaacttgttgccttggagattagcctgtcctgtcccacccccatgatctgtacttcctgtttctccctcctggcttccttaattaagccttgtgtgtacttgcgagcccatgtccatcacgctctgccttcaattctggatgctgtgctgtgaatacgcttgagtggaggccgcatgctttccaggaaggtgtgtattttcagtgttgtctctcatgatagtatgaaaggctctggagggaatcacaggcctccacaaacaggcaagcggaaaaatgttccacagacatgtttcagggacaacaggcttcccaatcctaggtcgtgctggtccataactggatcaggctgactctccctccccgacaccgctggtgtagacagggacaagaaagagaagtcagagacacaaagaagctgtgatttgcttgaggtcactaaagtacttaatggcagagttcagacctgacctgaggggcaacaaaagacacccccctcaaatccatggggagcagagtctatctgaatcagatcccagattttctggggctttcacgatgccaggtgtgtacactgagctaggatgggtttcccagggttctttgtggtcacacaactcaacaatacagaccagaaagcagtggcgagtaggctgaatttggctggtgggtgttttctgtttggtctgcacagtgtttaacttgttattataggaaattcaaacatgcagagagaattgtaccatcaacaacacacacccatcactctgctccaacaatgatcaattcaggcctatcttgcttcatcctataccctccctcgcttgcttcccttcccaccctcaaatatgtggaagcaaatccagagataccattccattgcaaatatttcattttgtatctctataatatgggaatttggggaatttcatttgagacaggatctcacttggttgcccaggctggagtgcagtggcgtgattgtggcctcaacctcccaggctcaagtgatcctcccacctcagcctcccaaatagctgggattaaaggtgtgtgccacatctggctaatttttttatttttttgtagagatgggggtcccactatgttgcccaggctggctggcctcaaactcctggtctcaagtgattgttttgcctcggcctcccaatgtcctgcaattatagatgtgagccactgtgcccagccaggaactttaacataatcctttttgacaaaaattttcctctaagaaattagaaaattttacagaaaaataaaaatccagacttctgtcttctcttaaaaatttggaagctctgccaatactgggcccagatggcaatcactgattagcactgagtagatgttgacttttcagatgaggggtgtgacgtctactttgccacagtccccaccactcccccacctggccatctcacccatacacatcacctgcctggctcctttaggcatttaatttgtgtaggaaattgacctaaaggagatattagagaagacattaaaaacttgaagaatgtgaaaatcagccaggagatagaactgtgctttaatgagggttatagaaccacactggatatgaagcattttgggaataaactttgaaaattggaagtaccagtttgtgaattttgctcagagagatgaaggtggggggtgggtgacagtggtgaagcatctcctatgtgccagtctcttttaccacaggctgtttctcacaaaatcatcaatcatatgcagtaggagaaacacctttgtattttacggatgagaaaactgaggcacagagaggtgaaataggcttcccgaggtcactcaggtcatatgagcagcagagctaggattcagacctggagttggcctgactctaaaacgatgcatgcctcaccatggcgcattgctcctttaaagagcaccagaagagagaatttgacaaagtggttgcccgtggggcctgggatgggtggggaaactacgctagggctctgggctgctgagggcctgagggaaggaatggggagcccgggcgcagaagtagaggagggggctgccccacggctacagaaggcatctgggaagacactttgctgaaggtggccaggagggcaaaggtcacagtggccattgttatatggtggtggtggggagagggggatggcttcttcttaactttctcgggtgagtcgaaggtttaccctccagacccttgatgggtcccttatgtcccttggacatcctgagtccccagccagggccctgggcgctttgctgggagcttgacataaggtccagcagcccccattgctacctaattccttagcagcccagggtgtctgcctgctacttccctgcctggaaggaaggccttctgcctggtgcgttcctgctgcagcctccactgcctcttagactctcacacaagctcagctccctgtcgcgtcctctcagggccaggcttctctttatcacaagcttagactgtctgagtaactaattggatttgtgtttgcctccacacaggaccctaagacccgtggggctgcgactccctctgtactggtcacagccgcaccctatccccttgcatggcgctgtcaaacccaggctggccactgactagcgctggaggggagcaaacgggaggccggccacctgactgatgttcccacaccttctccctgcaggctgtccccttttctgggactattcaaggaggtctccaggacggacttcagatcactgtcaatgggaccgttctcagctccagtggaaccaggtgtgtgtgtatatggatggaaacgtttcactccagcctcgtcccttagtaaaccactgtgcctgtgagcctgggttagttcaaacagcaacatccaagctcacgtgcctggctcaggggaggcccaatgcgtccctaacccatgccacagggcagcactccacaggcacaacctgcacccaggggtgccattaacctggactccaggatgaatggggccctgggagctgacggtctaaggagggcgtgggatggtcagagttggatgattatgttctgggttgaaccttgcacactgtgactaagagttgcaaaaatcaaacttcctctatttcccgtaactgctctcatccactctgaagctctccttctgctgatgtgaaaatattgaaaattgacattcaaagtaatttggtctctttcagtttcaaggccttttctggaaactactaagtcaattcaacatactaacattgtatcagggctgtttcccaccccctcctccctcccaaggtgggctgtctgggaacagagaagagctcacatggcttctcagtggctgggagggctctagaccctcacgcaggcctggctgacccctgccgaggcaccctgattgggtgacacagaggtgtgcagctctctcctggttgggtgggagaccacagggtggcttgctgaagtctgcggggcccacagaagagcctctggactcatggttctcccacagaagcctctctgcctcagtcccaggggtcctctccttaagaccttctgggccttgcagatcccaactcaacatcagttcggccactagattcttgctgacctcccccaggagatgccaagggcagggaggccctgatccccctgaaccctccaaccaggggaatcaggaccacacccctgatcttcctccccttcctctcttcctctcccttggcctcccctacctcccagcaccaggaaggcttccgactcccatcctgtcgaatagagacagatcctacctcagagggccctctcctctcgggagctttctgtctctcccttccttggggcaggaatttccaaaggactacagggatgaagctgaagggctccaaaagaaaaaaatacattcagaggtatttcaaatgtactcatctccatttattcatttattcatttaattaattattttttttcttttgagacagggacttttttctttgacccaggctggagtgcagtggtacgatcatagctcactgcagcctccaattcctgggctcgagcaatcctcccatctcagcctctggagcagttgggactacaggagtgcactgtgatgccaagctaattaattttttttaaagtagagatgagatctcgctgttgctcaggctggtcttcaactcctgggctcaagtgacccacctgccttggcgtcccaaggtgctgtgattacaggcgtgagccactgcgcccggccccaatttcacatgctctttacatttgttctcttctttcccacttaatcaggcttgtgctagcctccaacaagtgtccactggcatggaaggctctctcttctaggtgtgcttaacaacttccatctttggtaacaattggtaatcactttccaagacctatgcttcttgtttgctaatatcagcacttttccttaagcattttctacttatttttagaaaatcatactgtaaaggcaaaatccaataattgcacaaaaacacattagatccaaattagagaaacaaaaagatgccaaccaagcagtctctgcgacgcaagttgtatgcaagatcccagacaaatgcgaggcagaggcaccgaagccctcctgtgcctgtcactggcaataacccatgccacagaagactatttgctttccctgggcctaggtttgctgtgcgctttcagaccggcttcagtggaaatggcattgccttccacttcaaccttctgtttgaagagggagggtacatggtgtacaacacgaggcagaaaggaagatgggggcctgaggaaaggaagatgcacattccctccagcaggggatgccctttgacctctgcttcctgctacagagctcagatttcaaggtgagcaggaaacccctccccacctctcacccccgggtcccccagctctatcaggcgaatgggctttaagtaatcaccttgattgacattcagccagagtgatgctactatacagataacacaatcatttccttgtaaggtgttattcatggctgcctagtcatcttatgcacctttatctgaatctacaggtgcacctgctgcttcttttactctgaaaataagaactagaggagtcatcacgttgctgtttggtggtgtgtgtctttgaattccaagctcatgccattcctctgctatcaccacacctcctctccaggtgtctggtaacatttattattcagcgacattgttccagccttcatccagggtctattaagggtatagttttgtaagcaaattgcaaattacctttgcaagcagagtaggtctctgggcttcagaagcctgtgggtaagtcagtccgagtatgcttatctgagataaggatgtccccatccactcagtatgcaacctgaggtcacctctcctctgctccccatgagcattttatgcatatataaataatacccctgcagccataaaaaggaacgagatcatgtcctttgcaggaacatggatggagctggaagcctttatcctcaacaaacacaggagcagaaaaccaaacaccacatgttctcacttataagtaggagctgaacgatgagaacacatggacacaaggggggaaacaacacacccttgggcctggtgggggagggagagcatcacgaagattagctaacagatgctgggcttgataccaaggtgatgggttgatctctgcagcaaaccaccatggcacccgtttacctcgttaacaaacctgcacatcctgtactcataccctggaacttaaaatcaaagttgaaggggatgggcacagtgggtcatgcctataatcccagcactttgggaggctgaggagggcagatgacttcaggtcaggagttcaagaccagcctggccaacatggtgaaaccccatctctactaaaaatacaaaaattagctgagtgtggtggtgggcacctgtaatcccagctactcaggaggctgaggcataagaatcacttgaaccactgcactccagcctgggcgacagactgacattccatctcaaaaaaaaattgttcaaggaaaaataaataataaataaatgaaaccccaggtgagctaagactagaaagacaaacctactgcaggaaacaaccagaagaatgaaaagtccaactggagccccgtagagactcgggttgatgcacttcgggtcacacacacgttcctgtaactggccccacactgaagaccagactcaggtcttcattttctagaaagagggtccaggagctcaggggtggtccccatcccagcctgggatgcctcccccggaacacgtgctctcctctggcaggtgatggtgaacgggatcctcttcgtgcagtacttccaccgcgtgcccttccaccgtgtggacaccatctccgtcaatggctctgtgcagctgtcctacatcagcttccaggtcagactgtgcacctggcaccggtcccaggggctgggatgcagggcccagcatagctgtgtctaggcccagctgggtgggcccaagccaatctcctacccaggtcactctggggacaacctctgcttccctgtcccagtacctgcccgccccttctcctctgtcactctgcccctccttctgtgttactgtccctgtccggaacacctgccttggtctcccagactcctcagctgcccctttctcctcaccccttgatttccaccctggttaggaggccatattgttctagaaagaacacaggctaaggaaccaaactgaatccagttcaaattccaagctctgccattcatcctctgtgggatcttagacaagcaacttcacctctctcagcctaggtttcttcatctgtagaatggtcattgcctgccccccatgggtggttgtgtggttcagtgaggagacagatatcagaggtcacacagtggcccccctgggcccacaccagggtctgtttgacctctgtgttttaacaactttgaatgtgttgccaacatctaaaaggtcaggagagtcaggtatggtggtggaggtctgcagtcctggctactcaagaggctgaggcgggagaattgcttgagcccaggaattagagttctgcctgggcaacatagcaagaccctgtctccaaaaaactttgtttaaataataaaataaaataaaacaaataaaactaaagaaggtcaggagataccaataccacgtgaaaaatgggaagattagggtatgctgggcccacattcccacagggcaccagcagctgggtgggagctactattacccttttggcattcgaggctgctccctgagatcagcgaagcacacagtaggccctcagcaaatctgggcttctcgccctttggtcctcatctctgactttggtccctctttccctgactccctcccctgtgggtgaatggggagggaagagctggagggagaccgcacccctgcactgctcaccagagcctggctctttcccatcccatcttggcagggccctaacccccttgcctcatccccctgcctgcctggtctctccctcttgcccttgcctctgcctttcggcttctccttggctctattaatgcttctcctcactgcccggtgccttttgttttaacagaacccccgcacagtccctgttcagcctgccttctccacggtgccattctcccagtctgtctgtttcccacccaggcccagggggcgcagacaaaaagtgagttcaacacagaggccctgggtggccgagcagacagtaggaaggaccgagggtctgagaggctggccccagccagcaggccactcagggcctacaggccccatcttgcccactcaagagttccctgtctgtctgccgggcacccagagctggggacctgggggtctccctgaattcttcattcccccttctttacgtctctccaaggcaccaagaggcccgaagccaccgacatggcccaggcctccgggattcctcaccatgacgacaggggtccagttccccaccccccacacccaaccactgtcacagtggttcttataagcacttaagttcttataagtgcttcttataagcaaatctgatcacaccctctccctgcttaaaaatcttcacagcccccagcccttcaaataaagcccaaattccccagcagggcatacaaattcctcccagtctgaccccaatcaacctcgctgcctcacccctaaaccacaaagctcacgcagcctctgcagctcactcaaggccctctcgggccaccatgtttttgcatatgctgtccctggtcccggaagcatcctctgccagcctgcccccagcacacgcctaattgcccttccatgctctgcctaaatgtcgctcctctgggagtcctccccgcctggaatattgttggacgctcccccatgaccccacagagtctgtacgtatctgtgccatgccagttcaaagcaccccattgtaactgtgtttgcgttgtctgtccccgtcactccacccccatctccctatgcccatgaggcttcagagggccgggactgtgccttgttcatttgcactcagtgaatgaatgtggaatgtggttcacacagccagggagaatgggataccagccagggcaagaacagtctactgggtggggcaggatccaggacaaggaggtgagcagcccttcctctggccactccagtagtggggactgggaggaggggcgctttgtctatgcagtcttcttacggctcatcacggtacagacagggcacctgcctcctgccacgctgacttcaggactggtcgagccccagggaacatttgcagcgcagcccaaatttggccctggccctggggaggatagaaagtgtgctggatacagtcagacagaactggctgccactttggatttgatgccttccacctgggcgagcttgggcaagttgcttaatctttctaagcctcattacctcaccagggacacaggagctgaggctgcttccctcattggaaagcagtgaaacccaggaatcagcccacagtaggccttcaacaaataccacttctcaccttagtggtaaaatatggcactggaagtaatgctcttggctgtgggagctacagagagcaatgaggtctctatcaaacccagcctcctctctctcaagaggaaccaatggggataccctaccccccaaccccaaagccctgtgcacctgggggtaaaaatctgggtgccacgggctcaggaaggctttcttgggagcaagagggaggtgggtgtgtccggggaggcatttctgagcacaagagcctccctggagttttgccaccatctcctcccattctgtggtgcccgcgataaccactattctgactctcctcacccctccagcctcccggcgtgtggcctgccaacccggctcccattgtaagtctcttgctttctttttggatcgtcctcattttggcttttctgggctcatggaggaggcagggccaggcattgggcctctcccattgggagtggggagggcacaggccaggcccttgaccatctgcccggcctggtgaggttgggggttggatgtggttggctggccgtgatgaaacaacctgagttgccaccccgtgggcagccacggaagaccatgccccacattcacttctgtcacctgcaaagggaggctaggctgagagacgtttccccgagaggaaagatgggccagagccaccagcgtccccatctgtcttctccagggttctaacctttgcccctcgctcatccccttgagagaagagacacctgggcccaccctctgtggggtctgtggggccattgggcttgttacgccccctggagggtgcctgccgtgtggcgccctctggtgggagctggtggttttcacacgtgagagcctgggtgagacctggtttctttcttccagacccagacagtcatccacacagtgcagagcgcccctggacagatgttctctgtaagtctacaagttctggtcagttcacagctgcacagtgtcctccttcaccaagaactaaattccctagagcgctagagttgggaagaaagcggtttagcaaggaggatggggacactaggggctgagtgcttggctcaggtgacatgtggctaaagctgtccttgggacagcagagattctggaagaagcaggatttatccagtggtggaagaggcaagccccctagagaccgggagggaaactggtctgtctgtcaccaagtggggagtgcagtggccccagcacccggatgtgaggttcctggccctgcatcctgggtccccaggatgtcactaccgatcataggcttaagctcttctcctgctggtgcaaatcaggccggaagtttagcgttagtggggctgcctgcagaggggttgagggacaggtgaccgtggtggagtccgggaacacggcaggaagttccaggaaggctaacatgaaaagggaggtggatgggggagtccaagggccaaaggctcatgaggtcagcctcacagtggagcccctctagaatgcatgggtgcgcgtgggtgagtgctcgcgcacacatgcgctctcccattgaatttcctggtttcttttcagcagactcccgccaacccacctatgatgtacccccaccccacctatgtaagtgatttctcagggagggcggaggttctgtttgtggtgggcaggctgggggtgaagggccacttcgggggtatccactagccttgcaaaatgaaaagcgaagggctttcaaaatggtaagctgaagggcttcaaagcttcccagcgaattagaagactgtggagaggagctttcaaccttaaaactgtcgtgtcaatacgagcgcattgcatggctctcccttttcaccccacgagatgagtctttttgtgtttggccaaagttcctttcatgacactaatctaagcccattaatatgaggagcactggaaagttttcctttggcttttatggaaccaagtacagatatcgttggtttcttcttagctgacagcccaaattcatgggacctggtacaatcttcccttttgtgtggtgggctgccctggctgccactggctgacccatgcctctccttctgaaagccgacgccttttgtcaccatcattccgggagggctgtacccatccaagtccatcatcctgttgggcactgtcctgcccagtgctcagaggtaagctaagggctccagttgacctctgggaagagagagcccttcaaggtgattccagccattcccctggctttgggcaggctgtggatgacggggaggaaataaggcccagaactcagtggacaaaggtccaggtgggctgcccaccccaggttccacatcaacccgcactctgggaaccacatcgccttccacctcaatccccgttttgatgagaacgctgtggtccacaacacccagatcaacaactcttaggggtctgatgagaggggcacagctggggagaggtggggagtgggcagtgaagatgaagccccatgctcagtcccctcccatcccccacgcagctccaccccagtctcaagccaccagctgtccactcctggtgggaggtgacgtcctcagtccctcccctctgacctttaacctaactctcaccttgcaccctgcatcaacccttcaccccctcctggaaagccagcctgatggcatcccactggcctccaccaactgaccagagtgttctcttcaggggactggctcctttcccagtgtccttaaaataaagaaatgaaaatgcttgttggcacattcatgtgggttgaccgtggcttctttaattcatttggagacattttggagtctgtggaatcgggcggcggtgctgctgagaggagtgggatgggaaagcatggactccctttgggcagggggtcctgacctcacacctcctcccagagggtccctcacaaatcccacgagggtgttggagaaagcaggctggggctctagccttttgggcaggagtctcggacacaggaaggtggtaatgggggacaccaggaaacagccccatgcctccgagatgggagaggggctggggcagagcctgtgagggccacagcagggtcagagctgcctcctcagacctggcgtcccagtgggaggaggtggcttggggaggggggtcaggagaagccaacccgaagcaccgtctgggtgtgtgttccattgtgggtctctccggcattaggggttagggcttgcataggaaaagtggctcagtgttcatgtgtggggttctgccagccactgggccactgccaggaagcccgactctgcatccaggtgatgccaacacatcctgctcagcccagtaagaatccagttcctcaggccccatagtagtcctcagagcagtggcaccagctgcatccgggagtttgttggaaacgcagcatcccgcgccccacccgacctgcgaggtcagagtctctgttttcacaggaccccagacgaggtgcgcacattaaagctggaaagccctggtccgggagcggactcctggccttgttgctgagatgcacggctgtcgcgcagggcatggagctttcctggagcctgaggttggcactgccccttctcctgcccctgagatgctactgctagaaggggaaggtggcgggcaagggggtgaagtcggccagggcacccaggaggcagaggccaaacctaagctgggagccgtgtgttgccaggaccccagtggccgccgctgagctccttttttggttctgaccaggagaaggaaacggggcccactctcctaagaaggcttccttggcctgctccaaacccaagagggtccccagtccctcaggcgaggatgctgtggcccacactcgcctcgacacgctgccttcccagggaccacatccgtactggccaaggcacagtctcgcatgagactggtggccccagagcaaaacgctttctaagacagtgtccaagaagaagctcaaggccttcctcgaaacactggccttatgcgacaatcccaccctcaccagaccccgtttcagatactttgtgtgggaggcaggcctgcgccccctgccctaccacaggtctttggaccagcacgaagctccatctgctgctggcacacctggcagccatttcagaggagccatggactggctggggtgtccctctgtcggggatctgtccaggctgtgacacccaccagctccccactctgagaagagctttccctacaaaacacccacacacgcatacagtgtgattaaagtaaaatgcaattcatgtaaatatctccatcaaggaatcatacagggaagacccaagtggccatggggaacagactgggtgttagttttttaaatacagaggcataaataactgtacacaaggcagttaaatacacagtggtgcgatagcacctcctggtggtcacttgaagtggtgcactcagcagcaagttccatctttcacccacttgccaggcctggcatttaagattttgtctccaagggaccagggaggccccagtttgagagaggaggctccgatcaatccagggtgctacttgttaggaggtcaagtaaagggcttgatggggagcaacgttgaggaaataagacttgaggctgggggatatcactttcctccatctccccaggccctgtgacctcagataatgcagagctggctccatccttaagttctgtgccggcagctttaacccctcctgtaggccctcagagcgctgacatctccaggctgctgggctgcaccgccaccctgtccttcttcgcctcgtaaggaaatacagcaccaaagatatcttcgtgatagcgcttctggctcttttaggtaaaaacagagagcaacgtgtcaagtccaggctgggatgagcccctggggttgcccagcttccctatcacatggaccttcatgagccatctgtcttcccaactgttatacttagattgtgcttgaatctggtttagaccctaaccctgctcagcacctgctctggtgcgggccctctaaccatctccaaagctttacttagcatacccatttcctgcccagcgttcatcccccacccaagatactccaggaagtgtgagctctgagataaatatgtttgggaaatgctacgtcctttctttccttccagccttgaagggtcctgatgaacagcagcatactgaaggctcccagaagtcttgtagtaaagcaacgtgtttgccttgccttaactaagcatgtcccaggctattcgaccttggaatctttttgatgccaactgtctaccgacccactgaacacatttggggaaatgcctgttatctcctcactgttcttcaaacacgccagcttaactcccacccccggggcctttgtacctgctgcccctccagaacagcccagagtgtttccttcagtatccacagggctcctcctcaccatctcaggtttattctcagatgccaccttccagctctgccttccctggccaccctatttaaaattacactccccagcgttccctgggctgtttcctgctgtttttctctgaagcactacacctctttcttgtctgggtttgtcagccccagtagaatgtaaagaatttttgcctgattcatttactgctctatccctagcacatagtaggtgctcaataaatgttcagttgaacgtatgaatgtgtcttagggagagtatggaagtgacaatcacaggctttggaaccagagagaggcagactgaactctaggctctcacatttactgctgtggtccccgtaggcccccttccctctgtaaaagggggtgcctccctcataaagtcctcggcaggactgaatgaaatgtcacatgagaatccttggcaagcagtatgcccagtgtaagcccttgataaatatcactattatcttcatcaataaaatgcaagcccttcctgctggggggatgagggtgagcagacaccagcctgggtgtaaataagaagtgtgaggttcacacgaggctgacggggggcagccttgctctcccctgtgttcacagccttgctttggccacacagatactttctgaaaggggggtttggggagaggccagggagactctcctgcccccattctacagatggaaaaatggaacacattcacccgacttctcaaagctgccaactggtccaggatgtgcagtaaggccatctgacagcccacccagctcagatctcagccactgctaagattctcttggggtccagaacctttctgggcctgattcccctgggtctacctgccacccctggtcctggctccaacggcctgtgcccttggccggcaccttcagcccacacacccactactataccttgagctgaaagaaatagtcctcgacctgctcctcattcaatttcagcttggcagccaccagctgcttcagggtgtgggccacgtcccgggccatgcgcacatccccgcaaacatagaggtggcctggctccttgtggagcacacggagcacctcgctggccagctgctgccgcaggatgtcctgaacatagacctgaaaccagaggaaacagtgggttcagtcctcagctgctcaggacaaggcttgcaggcaggcctgctggcctcaaggaggcggggagaggggcactaaggggtgaggagtgagtccccttccagccaggaccttgcaaggggccaggctcaggatgaggtgcatggatgcccctggggctcggctgggattagagagataaatgctggacacagtcctgccctcgaggggctgaccatccagtggggatcccagactgtctctgaatttgccatgcttcaaaaaggcatcaatgaaggcagtccccctgcccagggctctgcctacaccccagaaaagggagcctctcaatgcagtctacccattaggtctctgcagggtatggatcccgggctccccagatacaaggtccaggagtcagtcagggactctctgctcctgctccacacaggatgcccagtggccccagcactgggcacagagcactgggcccaagaggtgccctgatgggacaaatgtgtgaaggccgagagtctattctgacaccccaggctcagggggtaacgcggccctggggacagaagagcccctctgatggccgtggtggaagcatcggccgccctccagccttgcaaggcttgagcgtagccagcctggaccacctctccccgggcagtcgcgtgtcccagggggcctgctggggaccttcagcaggctgggctctggggttaacccgcgtgtgacctaggttagtcctgaacctctccaggtctgtcgcttctgctgtatgagagggtgtaatgctcttcacagcatcactctgatggttgtgaattaccagcattttcgagctcaggaggtgaaggctcggggaggcgaggggcctgtggggacccagggctcacagtggagcttgtgtgtgtaatgcttcacctgctttgaggctgcatttaccttgggcttgccaggcaggcgggaataggctgtgtgcaccgcatgcagcacccccttctgggccatctccagcatctcctcctggtagatgtggtcctcatctgggcggcggcacccaaacaccaaggtcatgcggcctccccgcactcctgcaggagtcccgggctgttgttaccatgttggccaccagagggcgccagggctccaagcccaactggaattctcacttcactcccactagctatttaatagcattatccccactttacagatgaggaaactgaggcccagcgcattcagatcactcatccatagtcacagaactgaaagtgcggatgcagaattccctcagccaatcggagatcaaaagcagggactttcctgttacctccactcccaggagagggagccctggctactatgcgggagacggaaaggcagccattctaacccgcagaggcccgcacagggaagcaaacttgggaggccttccctcgagagaggtcaggaaccgtttgtggggctgcagttctgctcctaggcaggcgctggccccctggtgcccctcccacctgggaagcctccagccttacccttgtgctgggagtcatggagccgttgctgccagaaactgcggaagggcgcgatgcctgtgccaggcccgatgaggatgcaaggatgggagggatcctcggggaggtggaagccgctggcactgaagaggacaggagaagagggggccagtcctcagacaccccaggcccacgcacacacaggatggctggggagctgggcctgtgggaggcggtcgtgagggggtggagaggacaagaagctcctatttgatttcccacgtcctgcaggctgcttcacttctgtccaggcaccagcacctcccccaggtccttctgtaaaagggccaagctcactggactcttgagacccacctgtcatcgctgtccctgctacaggcagccacaccagtgtgaccccagtgccttctttatgggccaaggggctccgagcgtctcctaaaccccagatccctccctttcctcccggaactcccaggggtcggcggccccacaggggacagagtggaaggggtgcttacttccgcacaaagcagggcactgggtcttggggcttcaggctgttgagccatgtgctgcagacgccgtggtgcaggggaccctggccatctgcaacgataccacaaagtgaccaacgtccccccactgcccatgcgcaaactgtaccagctgctccgcccacaccacggccctccttggacgcccccactcaggcactctcaggactcaatgagcagctgtttgtcctcctggttctgagatccaccctctgtaagcgcggagctgaggaacgttgaaagggacttagagaggctgcaagttgctccttctgcccaagcagaaactgagacggggagaggaaaagtgacttgaaaaaggtcactcaggtcgtggtggaatcaggtggacagctcagcttccctgactcctactccagtgctccttccaaagactttctccgggaacactcccagctcaccactgagctccactgacagcagcatgaagtggcccctcagggtggctactgctgtgcgtgctcatgagcgtggcccttgctggtagcaaccctgggtggtggatgagttccgcgcatttcagcctgctctggcaccttgctttcctcctcagtgggccctcctgggcttgtagaaacccaggaggtctctctgggttgtgttccctttacaatggcccagcacttgaggaaggcctccaaccaactgcagaggtccccctggttttcctcgggattaaactttgatctcacaggacacgtctgcctctcctctcctggtctgggtggctgttctctagcagggcaaatagagtctctgccaaggttttagaaactgtgtttttgcttcttgaggacagagggctcggaagtgtgggcagctcccagctttgccaacccaggcaatgctcagttacagagggcagggcagggggcagcggccagtggtggagccgagaccacctggacgagatgttggcctcccgctctatggcagcgtcaccccctgctctgcttccctcagtgaccctgacctttccttcttggcttcagcacaatgaaaatcatctaattatttctatgcctatctctccactgccgcctctagatgctccctgagggcagggctgatctctgttgtatttccatacccaccccagtgccaggcacccactgaatagctgagtgagtgaatgaataatcaatgcagaatctcccctgcgttcaggctgcatgattgggaagactaagaaccactggcctcctaccaagggttgagtgctcttcacatctgatctcattaaattgtcccaagagtagtgggacagtggagtggtggaggacttggactcagcagttagactatccaggtctgaatctcagctctccacttactaacagtgtgactttctgtgccacctgtaaaatgcacacagtgcctaccccacaatgtaactgatcatatacatacagaaggcttggtttagtgcctggttcctagagatctctcccacacacctcctaatactgttaacaactctgggctgtgggtgttccctccctccttgaactgatgagggagctgaatctgagttgatgaacagatgtccaataatgggcggagcggggctgttccagagcctctgcccctggctcacctcgggtgtggtaggtgaccacggccacagtcaggtggatctctgtgggcgtgtgatcccgggaggagctgatggagtagaacctgggcttcagaatggggagctgggaaagcaggaagccagcagacacccgcagggacgggaactcctctagcacctccaggaatgtggggctgttggtgaacttccacttgctgtactctgagggctaaaagccaagggtgatgtcagtgactcagggcgcctgtcccgctttggggaaaagactgtcacaacccagtattcattcattcattcactcactcaacaaacaagtccatgtgccaggcactgtccatggtgctgagacgaccaaacttgagacaaaataagtgcctgtcctggaggaactcagtccagtacatttatctaacttgtgttacctccactgtcggcaactcatgggcaggggtcaaatctttattgtaatttatttggggcaaggctttggaaccaatcagacctgggttcaaatcttggctgggtgtcctcactagctgtggaatcttgtcccaattaccctttgtgaaacaaggacagtattgcctgtcttgtggggatgttatgggaattaaatgagaaagcatgtgccaagttagtacattgcagccacaaaataatggcggtggaggtagcttaaattgagataattagctataatgatgatgataataatgacaaccagtgccttttgcaaagacttggaaccagacagttgcaggtgagttaacgcattctctcattaacttattaattcagctctccatctgtcactctgcccattatgcccatttatccattcatccacatcatccatcccccaatacatcttaagtgacaagtttattccagttttggggaaagcaaagacggataagggcggctcccctttctagctaatgttcaaggcagtgcactctggttgcatcctgcagacacttggaaaaattcagttcactcaacacaactgtgtcctagacatgggccagcggacgatgcctggcttcccaccagcctttgacaagtctcactttgggaacctggtgtggatccctcttgaaagttaaaaccagctctggactctggctccccacatgctgggaccccccacatacccccactgcccaccagccctgatcttcacctggcacagggcctccagcctctgtctctcaggctcttctgtggccacctgggccagcttttggagcagcagctgggttgggggtgtggtgatgtccaggaagtaggtgagggcctggctgagtgagcaggggggcagcctcttgtcactgacccagtagctgcctggatggggaaggaaggtgtcaggatggaaaagaggccccattcaggattctcagcaggtagcgaggcccccaggcagacaccctctattctccagctgcaggggaaaggaggctctaaagacctggctctaagagcaaaaggcaggaagaaggtggcgcatggagaaaggactgccagggaaggctgatcaccagcccccaaaggggcagtaagttggcgtggtcctgataaactaaccacgtccttctctggcccagacctgaactttagcaagacactcactggtgatctacagactcacaatgactaaatccaccaaagactaatggatacctcacaggccagttgttcacttagagagaagcccatgactgcagtcccctgggtgctgtaccaagatgtgctgggtgagcagatgagcccaaagggaagtcccagggagcagcatgcagggcagtcacagcacagaccctggtgccagaagacacgggttcaaaccctggctccccacttctgaggtgactgaagcctctgggccttggcttccctccctgtgaaacagatctgatagcacaacctatcttgctgggttggcatgagcactgaatgaaattgtgccataatagttggtgccgtgcctggtacacagatgctttgtaaacactgggtaaaaataagagcctgacattctctcctctggaacagagtcctgatgaaccacatccttcaccctacagtcacaagggcaactgtcttgtcgtccccacgtctcaggttctctcactttctcctggacagtgggggtcactgccaggctctgtttctctgatcccactttcttttttatttttttgagatggagtctctgtcacccaggctggagtgtagtggcgagatctcatatcactgcaacctccacctcctggcttcaagcgattcttgtgcctcagcctcccgagtggctgggattgggataacaggcatgtaccaccattcctgtctaattatttttgtatttttagtagagacggggttttgccatgttggccaggattgtctcagactcctgacctcaagtgatccacccaccacggcctcccaaagtgctgagattacaggcgtgagccaccacacccagctcatcccactttctgactttggccactgcattgagggggatgttatcattagggaaactaaggccagggggccatgccttatgggctgggactgtttccacagctgctttcacataagctttggtggcccgccctctcagcaggaaggacagggatggctgtcaccggcaggcccaggctcagcccctcagccaggtggggcggccagagggtgccaggcagcactggctttctagcccggggctcaccactctcatccagggcctccaggcgcactgtctggtggggtgtggggccatccaccactcgctccaggataccttggaccagggccggctggttgcctgggcaaaccccaaggtgctcccccggcaggtagttcaggccttggccatcctcacaggagagttccaccaggatggtggcacggctggggaaggaaaatgaagcctcaggtgacattgcaggatttcctccagggctccttgatgggcaggcgagattccccaggaaatgtgccctcctttcattttcctggctggttccacaagctaggccaccaccctgagcttgggatgaggcatccccacaggcaggggtgcttaagagggccccaggagcttccctctggggaggaggggggcccaccccttcccagcagcccttggagccccccgagggcttggctacagcctctgcctaaagaaagcatccgccttggacatacatggatatcagaagaaacctttccttgttgccaggacaaaccctgttcttattagaaccaaggccagttttcctaatgcatgcggggaggacagcacagatcaatgaaacctgcagataatccacaaggctctttcccagagctgggaaatttccttccctgccaacacttttcctgaaagttcttaagaatgaatcaaacagtttaagtctctcttgcactctccttttagtgaaagagttcaatgaggaaggagaggaagtggagcagatgcttagagtccaagctggaaaagtggctcgtggttaaccaagagtagacgtaaaagcacaggtggccgcaggttaccaggtgggccggtcctaccgacagtgtggctgctaatgccagcatatgctcttctcctctcccttcaaagactgacttcttctgatctttcattcgttaaaataaaatcgacagagtatcacccacgaagccctgcactttcccttacctggatgtcggactttgtagattctgccgagatttgagcctcatggtgaacacgttcttggcatgcatgctgctgagggctgtggaggacacagagacggtgaaatggcaaagtggttcttgagcgtaggtaggggaagcccccagatgtctgagtcagtgacatctgaacaccacccttggggcatcctgctgaggtggccgttgggtttttctttttcttccttttattccacaaataaagatccttggtccaggccgggcatggtggctcatgcctgtaatcccagcacttttggaggccgcggtgggtggattactggaggtcaagagtttgagaccagactggccaacatactgaaaccaggcatggtggtagtgccaatagtcccagctacacgagaatcacttgaagccaggagacggaggttgcagtaagcgggaatcgcgccactgcactccagcctggacgacagaccgagactccgtctcaaaaaaaaaaaaaaaaaaaaaaaagctccttgatctgatacagggcagcttctaagtgttgcttctaggtgttgggaaattcaatttaggaacctaaaaatggaaagtcccgggacatctccatggcttgggatccacaggagagcattgttgacattggggatatcttaaatatgcagaaaccttcaggactatcttagacagggcacagggcacagcacctgggggtgcagagtggagagttcaccactacagcctggaatcgcctctgtgacaccttctctacgacacttggctgccttcgcagctggaaggctggggctcagaccccaacatggccacaggctagcagcttgcttcacctccttgaactgcaatttctccatctgtgcctttctcccaggaggagtgtccaggctcacttagcccatgtgggccaggagccccgcacagtgtctgacacacagtaggcccttggcggatgctgatcccttctgtctccaccaccctcctggggctccctcctgaaacagcctccctcagcaccttgagtattatgccctaacaggctcttgcatgcagtgagagggaggccccgaggccagctgtctctgttcagaaagacctgggggtctccttgaccatgggcttagggctcagaccccaacacaaacaagccccatgtgctgcagagaagcaggtacctttgctgaggtccaaaggctgtgagtcctgcacgagcctgtagtggtgcgggtcccaggtcacattggaggtgtagagcttggggatctgaatgtgctgtttgcctcggacatcaaacgtctcacaggctgcctggaagaaggtggagcagactgcggttaatggtcagcagcagcagcatccccaccactggggctaccactttttaggcccttaccatgggccaaacactgagccgtgtgtttcgtgtaacttcgaagcacacttacctgatagggtgacagcaaggactcaaagaggtgtctgggcttggcacatagtagctattgctactattataaatattgctttgtttttgttttgagacagggtctcactcagtcgcccaggctggagtacagtggtgccatcatagctcactgaagccccaaccacctgggctcaagcaatcctcccgcctcagtcccgtacctgtagctgagactacaggtacgcacaaccaagcccagccaattttctgtattttcggtagaggcaggtttcacatgttactcaggctagtttcaagctcctgggctcaagcaatctgcccacctcagcctcctaaagtgcttggattgcaggcatgctccactgtgcccgaccactatgaatgccaatattgacatgatcttgttgtatcctcatgcccacactggaagaggtctgattgctcttatgttcctggtgtggagccacatggaggaggcctgggctttggaaccaagcctgccccgatgcaagctgatgctcctaagcaccaccgctgagtgacgctctgcatgagtgacgagttccctaaatgactggggcaatcacagcaataaagagctatggagatactgagtggatccaaagtggatccgccattgtgtggtgcctgtccccaggctctctatggctgtgagactgaaacagacacagtagttaagaggtgacctaggtagtcactggaggaaagaccttaaactagaagctccctaagggcaggggggtatctgtgccaccttctcccccagagttgtgtgcacaaggtggagctcttctagggggctgagggagagcagcagtgacctaagccctcaggtttgtggccctggcccatgcctgggaccacatctacaggaccacaggcagaggtcatgccacctgggacgcccagcacagatgtcctaggcatgaatgcaccctgtcatgtccctgctgtggggcagctctggctgggaactgaccttgaaggtttgcacggcccagctgcggaaggcgtcctcctgcccactgagctcatccccttctcccatcggggtgagctgagaggcccccaggtgggacagcttctgatcaatgtcatgagcaaaggcgcagaaccgagggtacatgctggagccgaggccaaacacagcgtacctgcccgaggacacacacagagacacatgtcccatgcaagcagcacctggcacccagcacccagcaccaacagcctggagagccagccccagactctccccctccagctggagaatggagctggacccccttctggtcctctccatcccccagccttgtcaccaactcccatgaccaccccatgcttggcctgcagacctgtgagagggacttggtttctgtggtttgaccctcgacacattctgagtacagaccttagacccaggtctctgtgcctgcacggttctgagaagaccccctgtgcacacccagttccatcccctgaacccagactttgggtccacagggcagggctaggagtaggacaacggaaaaagctttacctgaatttgttgttgagctctttcagcatgaagagcgatttcttcagtttctagaaagagagggaatgacagagttctcaagccaggatgaataaaaacactgttttttcctttcttctggaaacctggccacagcttgcaggagaaggctcacagcccaccacggaagttggtttacatatggggctacctggccaaggacctggttgtcagctgacaagtttagcttgtctctcactccaactaaactgtagcaacttgtgtaggccctgcctcttaatgggggacctggaacagctgggcccccacccatccagccctggcagaggggcctgcctggtagcatgccttctccagaggggctccgaatgcgccgtgattctgtttggaacccggtagaagcaacaacaattggtcctacagtctctgaaataaggctggctttccaggaagccaagatgattccaggaggtatgtgacccagcagaactagcgtcaaatcccatggtgagtatttactcccttttcataatttattttttagttattccccttcaattacattttcattcttcggagcactggagagacagtctcatattggtgctaatagacctttaatacctctctcattgcctattagtcttttttaaaaacaaagatagctgagcacggtggctcacgcctataatcccagcactttgggaggctgaggcgggcggatcacctgaggttgggagttcaagaccagcctgaccaacatggagaaaccccatctctacttaaaaaaaagaatacaaaattggccaggcatggtggcgcgtgcctgtaatcgcagctactcgggaggctgaggcagaagaatcacttgaaccagggaggcagagtttgcagtgagctgagatcgcaccattgcactctaccctgggcaacaaaagcaaaactccacctaagtaaataaataaataaataaacaaacaaacaccaaaaaccaaaaaacaaacaaaacaaacaaagagcaggccttggccagggaatagtatctagctaaaattcattaacttcattttgttttcattgaatttatgttttgctgctttccatttgtgagagtgatacaagtttcctttaaatatgaatttgtaaatacaaacaggtaggccattcacagaaatatattaaatatgtcataggaaaggtggcactcccatatggcaataattacaatggaggccggcaaatgacctgagtgacccggagtggcctgagcactgactcccaaatgtcctccgtaggacctgccgcatcccccagaccctttcctgggtcctcccgtgccctaccaccccctagaccagccagacctcaagccacccacctctccattgccagggcagtctccattgccaaacgtactggtcaccaccaacagcagccgttcctcctccaggcagctcagcctgtacttatccatgcagacaacctggatggcacccaagtggacatcagccctcggctcccagtagggccctccctaccctgcgcagacaccctgggcttcctcccacactgcccccaggctgcggctcctgtgctcatctgtctctcccagggcccggcacaggcgtggcacagaggacgtgaatgaaccaatttgaacacatcatcctctgttctccttccccgcttaagccctatggttaacacattggcaagccctggaagatctgtcccaaaaatactgcctttcccatgtctaaatgcctctcaccatgtccactactatttgcaattttgtgtgtgtggaaatatttccacaaaattggaatgagcaggtcacagctgtgcctggagggaatggccggggaaatgtgccctcatcttgctgttctatccaggcccacccagcggaggatgggggacctgccaccactctcctggcagttccagactcctgggagccagcaggtgaggcttggacccaagaggtgttttcagcaacctggccgacctggtcatcagtcagtgcccccttggcctaagcctctacgtggcacagaccacagctcatcccatcgtggcattacactggcctgtgccctgtcctcagagtcacatccatctcccagaagccgtgcgaccctggaaaacccatcaacctgtctaacagtcaggttcctcctctgtgcattaacaatggcattgacgcctgctttgcagggcgctgggaggggagggtggggtgtatgctggagagctccccaagggcaaggcctggctctgcgccacccactgtcagatcctcagagcccggggctggtccagcacgtggccaccattccctaagagttggattttatccatcagtgctgaaggcaggggatagagcttagacagaccctctgcggcctttttttgtttatctgaagcttactcatctcttccccctttcacatgcagcactgcatcctgcagagcaggtgttcagtgagcctgaataaagttcagccagagcagctgatgtatgttgaggcctggattcattgtgaaagtgtttctcaaactgtgttctccagaacaccatgaaaaactcatctactgtgtaagttttggaatccctgcacacaagtctttccttctcttccagattaatgatgcacattaccacatccaagactctgagacatccaggagtggggaaaccgttttaacttttcctagactcccctgcacacaagcagaaaaaacaacagccatcccactgagccagtaccttggggttgaaggcacagctgaataaggcccccaggtcccaggccagcgcctctgattttcctgtctctgtcgcaaagaggatggtgactctgactcgggacgccattgtcttgcgcatcagcatacaggcaaagagcacagctctgtggggacagacagacaggcaggcgctgtgtgctgagtcagccttccagaagaaaatgggggaccaggggaatgggaggggacagcgtttaatgtggggcaggttgagggaaaagcaagctacgtttctcaccagccatgtaccgtgacaactcacccttttcctcccctcacccactactataggcccctttgggaaaagggaggagagagaaggttggaacaatggcagcaggggacccttggaattggcgactggacctctgggagccaaattccacgaagaatatacattcccaggctgggtgccgtggctcacatctgtaatcccagcactttgggaggctgagcaggaagatcgcttgagcccaggagttcaagaccaacctgggcaacactgcgagaccccgtctctacaaaaaaaacaaacaaaattagtcaggcgtgggtttgtacctgtggtcccagctacttgggaggctcaggtgggaggatcacttgagctcgggaagcagaggctgcagtgagctgtgatggcaccactacaccccagcctgggtgacagagtaagaccctgtctcaaaaacaaaagcaaaaagcataaccattccttgtgttcttgcccttcaaggactggaggcagaggctgactagaagggagagatatagttcacacatcactactagccactgccactgccactcacttgaccaagactttcaatggaatctctcttctcttgggtctccgcttctcgtcctgccagacatgggttttccaggcctctaccttcagaaaagaaaggagatgtgagggcagggcggggtcctggcttggctcagctcgcggatgctggagtggtagaggacccttcacacccatcatgagcctgggagagaaggctggcctgcgccccacccctgtcactgaactcccacaagggagggccgtggctgcctctcccccagagttgcctctccactgcccggcatggagcctgcacatcgagggggggcggctaacccttattctcattagtgtcctcctggaaggaacaggctgcactccttttgcttttagcatcttatcagcaggcagatgacataatgtacagtcccaaccgagacacctttgagagcaagaagagctgctactacaacaggcaagactccaggacatgccgtcaccctgcatgtgaggtcctgtagatggaggaagctcttcctttagatgaaggaagctcttaaaatccaacccaagtcctttttggcacaaaatgaactcttctcaatagctcatggttactgaatgcattctgcattgggcactgggctgagctgttccatacaccagctttgtgatccacacaaccccccgtcgtggagtgaagcccatcccttttagagatgagaagatcagggctcagaaaggtgaaaggacccacccaagaccctattgctaggaagtggcagaatcaatagcgtctgactcaggggccaatcccctcaacactctgctgagtgactgttgccgggccctcccgtggaagcaggtgtaatgacagaccccccatctgagccacaggccactagcggtgcagtggaagccacttccttcagtgccactctccgtggaaatgcacaaaggctgctcagcatccttgagtggggcctggaaagctctaacaacatactcctgcttcccgtcgtgccctacatgtgtaacaatgaggtgcacacacacacacacacatacagccacatctgagcccccaggaaggagagaagaggaaggcccctaacctgatagtagtagaaaggggacaggacgtagttcagcatctcctggtgaaacacgggggtgatgctcccagacatgggagggaccagccaaatccagtctgccgggcagcccccacgggaccggtattcattctgcatgtacttcatgaaggattctgcagccgagtggtggtccatgatggtcacattctgcttctgtatcagaaggcaagatagggagtggagataagggtgggggaatcaggagaggggccggttggccgcagggctcccagagagtgcctgggaaggcctggcaatctgtaacacgggagagcaactcctggccctctccttgcctgctgaaaataaacacccaaatggaaggcgcttgtggagcctctgacagagcccaagaaagcacattagtcagggtcagcccacaggtcccatgggggcctcccttaagtctttgagggcaatatccagcctctcatgcccacaggtctccccagactcccagcaaatggctctctctggatctgcataacttaaaccacagcaccttttggcaggtaaaggaaataccatcccagccatttatggtggagagttctaagagtcacttccatggggttgtagcctcctcagccaagaaggagccagacccctccgtggggaggtctgaagcattgggagaactgtagtgtatgggagacgaatcactcacagtctgaaattacgggctgtggggtctgaccccatggaatggtccagcaaggcagccactggcctctatgtctgtggcaacccattcagtcaagagacatttgtttaacacagtggttcaaactctggctttttatttgccatgtgaccttgagcaattagttctttaagttttctcatctgtgaaatgggagtgatatgaattcctataatggccaggtgcagtggctcatgcctgtaatcctagcactttgggaggccgaggtgggaggactgcttgagcccaggagtttgaaatcagcctaggaaacatggtgagaccctgtctctataaaaatttaaaaattagtaggccagctgtcggggcttacacctataatcccagcactttgggagggcgagacggggagatcacctgaggtcgggagtttgagaccagcctgaccaacatggtgaaaccccgtctctactaaaaaatataataattagccaggcatggtggaggcacacacctgtaatcccagctactcagaaggctaaagaaggacaatcgcttgaacccgggaggcggaggttgcagtgagccgagatcgcaccactgcactccagcctgggtgacagagcgagactctgtcttaaaaaaaacaaaaacaaaaaaatttaaaaattagccaggtttggtggcacatgccagtgttctcagatatgtgggaggctgatgtggtaagagcattgcttgagcccaggaggtcaaggctgcagtgagccatgattgggccactgcactccagtctgggtgacaaaacaagaccttgtctccaaaaaaaaagttcttacatcatgaggttgttgaaaggatgcaataagctggaataagcaaagcatagtaagtgcttaataaatattagctgctcttataatctccatctgccaggcactgcataaggcatcggcgttatagaagggaaagacacagccctttctctgcattagagtctagtctaactgggtggggaagccaaggaaaatcagtcattttcacacggcctgatagaaactctgatggaggcaggcaaaggtgctggggaagggcagaagagaggatacaacgccagacagaggcccagggtgagttggcgaatttcgggagcagctcctgagaggaagggagacttgtgagaggggtgcaagttagttgagtggggcactgcatgagtcaaggcagggaggcatgagaggatccagctggtgtcaggcctgggaaggccagccatgccttgtaaattcaatagacatacataagttatatatgtgtatataaaagtccggccaccactcttgtggacgtgggctctcattcacttattcttccaagacacagcaactcctgggagccagatcagttctaagctcagtggtagctacaggatgggtgcagtggctcacacctgtaatcccagcactttgggaggcagagacaggtggatcacttgaggccaggagtttgagaccagcctggccaacatggtgaaaccctatctctactaaaattacaaaaattagccgggtatggtggtgcaatcctgtaatcccagctacttgggaggctgaggcaggagaattgcttgaacccaggaggcggaggttgcagtgagccgagatcacaccatgcactccagcctaggcaatggagggagagttcatctccaaaggaaaaaaaaaaaaaaaaaaaaaactcagtggtagccacgatcatgatgacaacataataaacagtaagaaaatcattgcttactgagtgttgtctatgggccaggcagtgcagctaaacattttgcatgcatttttacttaactcttataatcctcagatgagggaggcactattatccccattttacagaggaggaaacaagggcccatcttttgaagtttaccctgaaggagggtggtgaggaagtcctggagacgctcaggcagtggaaggacacagtcagccacggtgaggagacagcagaggctcacagattttctgggacgtatccacatcctgccagcatcagggctggtgctgggagtccccaggagtcaggatgtgtggtgcaaagccagcccttccccaaacaccggggggctcttcctgttccatcttcccctcccccgtcaagcacaggcctgatctgcctatggggtgacccagtcatggggaacagtgtgtgctcatctgtactctgttcaccactcacatccttggcccagcctagtgcccagcaccaggcaaatgctgagtaaaacactgctgaatgaatgaatgactgaccagtggtggggtctccagggcatctgtcagctttgtggcaatggctgtggcttctaaagtagtatcttcctacatctcaggcctccttcccacagggaagggaagtgtttctatgagtaagagcagcggatgggcctgagggcttagccctacacaggagcccagcggctgtgtgtgggcttaagaggaggtggaggttgttatcctcgcacagcaagcaagagcagccacagctcacaggtcccctcccagcatcactcattcatgcacacgtcagacaaggactgaagacccagtttgtgccaggccagatcgcggatgtggccctcatcattcattcttcaaagacaaattattttagcatctaccatgtgccagacactcttctaggccagaggtgcagcagcaaacagacagacaaggtccctgccccatggagcttacattctagtgggggaaaccaaacaataaagaaagctggcccggcaccgtggcttatgcctataatcccagcgctttgggaggctgaggcaagcggatcacttgaggccaggagttcggaaccaacctgggcaacatggaaaaacaccatctctactaaaaatataaaaattaggcaagcgtggtatgcacctgtagtcccagctactcggcaggctaaggtgggaggatcacttgagcccaggaggtcaaggctgcagtgatcatgccactgcattccagcctgggtgaccgagtgagatgatgtctcaaaaaaaaaaaaaggaaaagaaagaagaaagtcgatgtcctatataatgccaggaactgtatgaggaagtattttgaagaaaaataaaatggttaaaggactaacacagagagacaggttggcagtggggtgagggcgtcattttacatggcacggtcagggaggccttcttttttttttgagcggagtcttgctctgtcgcccagccaggctgaagcagttgggggatgaggaagcacaatgagatctctccatagttttgctgtagaaaggagcagaagggtgaggcagtggctggagggagcctgaggtttggcaggagttatgtggttagctattcatttctttctttgaaatggggctaagagagcctgtctgtacactgctgggaatgactcagcagagagagagacattgatgatacagagagagaagaggaaaatttttgggagtaaagtccttgagcgattgagagaggacagaattcagtgctcaagtacagggattgaacagtcttgggaggtgaggagctgcctgttgctggacctctagagtgagcaagggggcttacttataaattaagcatcacataagtcactggatcagttaagcttctcaccaaaaagtcttcagactcacaaaactccagacatacctggaaactatggagcacagcaatgttgatctcaacgacagcctggtctttccagagcgaggccagcttgtgcgtttccaggcccattctcctgcccacttcctacagaggcagagtgatagcggcgagtcggtccctgaagccacccccaggcccacaccttcatctggccagctgggctggctgggttacctccaggatgttgtagcgctggacgtcacagaagtcccggactccgatctctgtgcccatgtaccagccattgaaggggcaccctgggaactccaggccgcccacctcaagcagcatgttggccactgcaggcagggcgtaccactttagctccagttcccgaaaccactcgtatctggcaaaaaggtagacacaatttaactggggccttccttatttttattttattattattttttttttagtgacttggtcttgctctgttgctcatgctggagtgcagtgcagtggcacaatcataactcactgcagcctttaactcctgggcccaagcgatcctcctgcctcagcctcccaagtagctaggactgcaggcatgtgccatcttgctcagctaatttttttccttattattattattattattattattattattattattattttatagagtctcactatgttgcccaggctggtctcgaactactggcctcaagtaatcctcctgcctcagcctcccaaagtgctggaattacaggtgtgaactaccttgtctggcgctgggggcttcacttttgcacctttggcctcccactgaccccccacctgcccaccaagaaaaaagcttcagaaagagtcaaacaagaaaactcctttccaagagttgattctgagttaatcccagccagagagacgtattggacacataggatgccatttattaaattatgcattcatgcactcattcattcatgcaaacctatattaagcttcaagtatgtgccaggctctgtgctaaatgctgagaatgcaacacaacagagcaattaaggactccagtgtcagaagctggggttcaagttctgcctctgactagcagtgtgaacttgggatgttttttagcctctctgagcctcaacatccccattatcagaatagaaacatagcatcaaattcatggaattattataaagctagtaatatcattcacttgtatattcagttattccacaaatatttataactgagcacctactatgtgccaggcactctgctagctgttgggtatacattgtgaacagaaaagaaaagatctccaccctcaaggaacttgtagtctaatgtctagactctagattataaatgggtaaacaaatatataagaaataaaaacaagatgatgtgttagactgagggtaagggggcctcctctagcttgagtggtcagagagccttctcagaggaggtgacattataggaaataccttttgacaagaagaaatgagcctagaaaaattctgaggaaaaggcatgtcatgcaaagggaacagggagtacaaaggctgtgagtcaggactgaatttggttcattcaacggccaggaagaagagcccagcccaatgtctgctacagagtaaatctcaatacgcagaggtatttttgcaattatgagaactgcctagagcaggggctgggccagtaggactcttgaagtcactgcagccctatatgatcccgacaggagagggacagctaagagcattggcaggagggcagaagaggcctgggctgctcctttctagaatctcaatgcattcctctcctgcagtcactgcttctcaagctcctggggccccatccgcctaagcctctgcttcccctcagcatcaacacatccacctgtctgaggcccgaggtgccagtgcagaatggctgcagaatgcaacagtgacaaaagagtccttgaaggccagccccctgagtgtcacctgctgctggctgggctttagggaaggtatggggaaggctggagccgcacttaggaaaggagaagggctgtgtgttgactcgcccttgcccccagcaccctcagcctacttacttgggatgttccatggccacctcaagcacaaggtcaggtgggatttcgaagagctcagggtcacggccattggcctgcaggaccagggggaccacatcgaagcggccgtacttgggcttccagcccaggtcgatgcacagctggggaacaagacgggccctgtgagtctgtaagcccgggctgcgtgtctcctctgggctccactctgtcactcgctcaccacggggctccccgccccaatggccggtggctgaggctgggccgggtacctgagtgaattccacgttggcagggtcccctctgatgctgccatctggcatctggtagccagcatagcggatgagctgagcattccacacccggaagtcgtgcttgccatcactccgctgggggaacacggtgatggccgaccttcccaggacaggaacagtactgtttaccacctagccctggtgggggcccagccatcctgcactggccctacctccctctccaccctacctgctctgcagcccctgatcccaagctgactgagccagcctcctgacatgccttctcctgctgccctccctcaagacccagggtccaccccctcaagggggccttcccatccatcaggcagggttagctgatctgccagtctttccctcactgcctatgtctccgtccttgcagttgagttagctggttgcctgtctagtgcaggtcataggctcacaccagctggagcagcatcttagggtcctagtcactcttcctcctacccccccataccccatcttccccctgctctcccagccttgtcctgtgttgagttgcattaggtaggcactgagtgtttgctgaacaaaaagatgaatgaatgaccaaatgagctgttcagtcctattagctttgacctcgttggttgggagaggaaggtaacctcttaatgtcccatgccttctaaaaaatgggagccccaaccctgaatagtgaagcagttacttcttaaagatcctccatcccctccaagtggctccacatcagaaggaaaccaagggcatgggttgccgatgggagctgagctcacaggggctgggctcactactggaagtccccgaagaagcctgagtctttctcttttttctttctccctggtttctcctggagctggagatgcctcccctagacccaagtgctgcatcttttggctaagattctcaggcaaggcaagcccctctgcagccctgggaaatgtgcaccgacctgatgttgccattgttggtggagtaacgcacgtgtctgcagatgtgttcaaacatttcccgggcagtggaacagctgcgggcatcgaagacctgcaacagcccatccagaccatgcccatcaaagactgggtagacagaggctttgaggctgatcagaagtcactgggaatcaatagtgcagccgaaacactcaacacacaaacactcaacaccatgccgaagagtgcagcctccaatctgcacctgccactcaacaccatgcagaagaattcagcctcccatctgcaccaccgctcccatttcatggctgccctctaggggagcacaggcaaccccactcagaagaccccagcaaacccagaattccaagaaaacctgacttgaaagcaccatgaatcgggattgccacagccccaggctaggtcctttcccacaccagcacatactacacagccagagttggtttcactggcagttctaatcgatttctctgtccaagtgcttttgatattatcctgcaacaaatgactccccaacaaaaagatggcctaggaggctaggctaaataaaatgttctcttggagccaagaattcaagcattggaggaaaacaacacatcttgcagcaccacggcctcaagtgtaataaaaacaaggtcgtggtttactctaatgatggcatttagtctcctgaatttaacacaatagagaatgggcagctagaaaccctggtgctgaaacaggattcaaggcttctagagagggaccctaggtgccccatctcaacatttgggaatgtaagagaggatccagggccatggcttcagtcccaggaggcctgggcacagctctgtgtggctgttccaagccatctgctcagggcctggccgcctccagctctctcccgctcaagtctaaggaagttcacctgcaggttggaccactggatcctcccaatgcagcgtggggcattgcgccaggcctgcttggtggcgaagatgagctcatctcccgtcagttggtaggttcctgttgtttctatctcctttgttaccgcttccaccctggccagatgttcctctatttttgccctgggggacaggaagacagcaggaagatcaacaatgagatggccttacatggggattaattcaatccacagaagcaggaactgaaataggacatcagaagggcaagatgcctctcaccagagacagctccctccagggctggctttggggacctcacacttagcctagaagcaccccacgctcaatgctctgctgtccccatcttgaaattcttaatcattttaaaataaagagcccacattttcatttttacattgagtcctataaatactacctagccagtcctgagcccatgggaacactgcttattttcaggtgctttttctttaatcatattcatctcagtaaggggtgcaatccttctcagacacttgcaagcctgggaggagtgtcccatccagccatagcaatgtcctgcctctccattagttagtagagtgccctccttccttgccatgactgagagatgagccagtccctccgtggcagctccggtgtactgggtacccacatgagccaggcactgggtgaagcaacccacaggcagggtctcactgagtctgggagggaggtatttttgcatctattttatagccaaggatctcaaggctctaggaggttaagcaccaggcccaggaacataagtacaggaaagagcaaagtcaggactcagacccaggtcgccctgatgccaagcccaggttcttaactgtcagcaacgtgaccttccatcttccaccctacggggacagtaagcccaaagatgttgagggcctgcccacccttctcaggagcctggtcaagccaggactgggacctggctatcggagctcagtctgcagggtcccagggcctggccagaaaagagctgcccacgtcatagtctctctaaaccgtgctgttcctcagcctcagaattgctcattcagtcactccatatgcaattctaaacttgcttctctcctcccctcccagacttcaaaaatatcccaaagggagggagggactcagtcagaaaggctttgaaacacacacacacacacacacacacacactaccaccaccaccaacaacaacaaccttgagatttccagtcagcaccaaagccttttgaaacagcatcaaaactatttaaagaatgaactcctgattaaggagagtgggtaactctgaacaaaatggcctttctgtggatgaagagtgtctcgacaacttttccttgattagagaaaaaaagcacaaagacaaaacactgatgatatttatccccaaggacacccctgccataaggagctgctgggtgaggtgggaaccaccccactgctgggagacctccctccccaaacaaagcttccagcaatattcccccctctgcacctgagcatgacaacagtaatgatcaccataacacccacaccaccaacacacatggtggtcagcacgtgcatatgcaagaaaggattattttttacattcacatccaattccttcagagttttagatgtgatagccattttcatatagtcattttctaaggcattagaaatgctatgatgtctatgagctctgaaaaaaaggagtggctttcatcatcagtgaacagatttaaatgtaacatgcagcaagtcacaatctctaaatatgctatgattcttcagatacaaaaggccactgaacctgctcagctggtcatgtcttagattgccagggtccctgtgaggagaggctgccattccccgttcacacccccaggttctcttgctcagggaagcaggggttcaaggtatgtacttgtgacttggaaaaatacaattttcctctgggagaagcaaatgggttccaacaccaccaagcatggccctgtagccctgctgggcttctgaacacccgcccaattgccttccacacaacagcaaggcctcccacgtccagaatagagttgggaacctgcaaggccacacagctaggtactagagcattgatgggaaaagcaagggctcaggcaccagctgcctaggttcgaatccccactctggggcttcgaagggtgtgactttgagcaaggtacatctcttctgtggatttcagttgccttgtctgtaagatgaggataacaataataccaatctcatagtgtcatggtgcacacgtgctttatgctaaataaataaataaataacataagtactgtggcccaatgaaacccatccaggaaaatccctggaccccgaaaggctcattaattaacaaccgagctgaaaggccaaggatcttgtgaagtttgcagtcaggaagcaggcagcccctaacaggctgctcaatccaggctgtgcattagaatcacctggggagcttttgaagcacacacatgtctgcctcatccccatctccccagattctgctgcccttggctcaagggaccctgccatcagaattttgcaggctttccaggtaagtctactgtgcagccagggttgaggaccaccgaccctggaagatgagggcctcttctgaagactcacggtccctcccagctcagacattctagggttgtagaaaactggaggtgggccaggtgcagcggcacacacctgtaatcccagcactttgggaggccaaggcaggaggatcgcttgagctcaggagttcaagaccagcctgggcaacatagtgagaccctgtctctactaaaaatttaaaaaattagctaagcatagtggtgcacacctgtggtcccagctcctcaggaagctgtggtgggaggattacttgatccaggagatcgaggctgcagtgagcagagattgtgccactgcactccagcctgggagacagagcaagaccgagtctcaaaaaaaaaaaaaaaaaaaaaaaaaagacaaagaaaagtggaggtggtaccaccagaagccactctttccgccagggagtcccaggtacatgtctgtggctcactttgtgtggtgcagtgctgtgtgaggctctcccctaatagcaacactccccttccagcctagaaaacaatccatttctgggccgggctcggtggctcacgcctgtaatcccagcactttgggaggctgaggtgggtggatcacaaggtcaggggattgagatcagcctggccaacatgatgaaaccccatctcaactaaaaataaaaaaaaaaaattagccaggtgtagtggcacacacctatagtcccagctactcaggaggccgaggcaggagaatcgcttaaacccgggaggcagaggttgaagtgagccaagatcgtgccactgcactccagcctgggcaacaagagcaaaactccatctcaaaaaacaaaaataaaaacaaacaaacaaacaaaaagaaaacaatccatttctttcagacattctttcttacaatctcttcatgcatttttgttttaaaagttttaaaggggcctaaattacatagccacatattttatttggacaacgcagtgttctaaaaacatgaaaatggctaatacttatgtcgtgcttactgtggcccaggcattgttcctagctctatattctttaactcctttcatctttataaactcccgccaactaccctcctcctgcaaggtgggtactattattatcaccattttcggtaagaaaatcaaggcatagagagacaaagtaacttgcccagggttactcagctcgtaaggggcacagctaggatttgaacctgggaaggccacaatgctatacaacctctctttggatgattaaaattgtatacgttggcaataaaattggcaaatgacaattaaaaatctttgtattccagtgtctgacacaaggctgaggcatactcagtgcctgatgtgtgtctgtcaaccaaatgaccaattggccatagaaatgaaggatgtctagcatggtcctcagggaagcctcactgggtagaaaaaagaagagaactccagaaatgtagaaccccaccagggtcagggcaaagagcaatggcctgccctctcctagtcaggtggcctggcttctagctggaatgaggtgacttcaggtatgtcaccagcttccatgttcacacctgtaggagtaggggcctgactaggtggtctcagagatcctttctgtctcaaacatttcataattaaagggtccatattaattcacaatcaaggtctctggatgccaggacactgttctgaacttactgaatcctcacaaaaatctcatgagccaccttctgttattatccccattttatattaggttggtgcaaaagtagttgcagtttttgccattaatatgatgaggacgttcagacacaatgcgggagccagggttcgaattcagtcacttttaactggtattgctatgtacaacgttatcccacaattatgagaacctgctccagggtcttctcccctagaaaaccgtgattttcgctctgcaaaatcatagtgatttaacaccacaaatctggaaagaaaagggatctagctaggatcagagggtgatgggtagagacaggagagcaggaggaagggcaggaggcagcgaccctgcaggaggcaagccttactctttgaaggagccgtaatattggttgacaaattcgatagcttgaggtagaagctcatctggaggggtaggcttgtccctgggtcctctggtcaaacttttgggagtcataatggaccccaggcaagatttggacctgcaagttaaaatctggaaagagagaggcaggtggtgagggtgggccattcggcctcttgccaccctcctctggggccagctctcctggccacctggctcctctctggctgctccggctccctgcacctcttctccacccctgtggcacctcctccttggggtccacctggctttctcccacctagatctcctctcttccactggctgaaccccagtgggtgtgagtcaggtttttctccctactccggactcagtctctggccctgctccccagccagctccagatccagagcaaagtcaatccaatatggctcctatgggaacatgatattggtatttggccaaaacaaactgaaatgagacataggaaggcacacgcgtgcacacacacacacacacacacacacacgaatgaagtacacccgaatgttaactgtggtggtttgggggcatgtgggaaataattttgctttcatggtcctgaaattgtccattctttcccactttacaaccatagaatgtatcctgaagttagaattaaaaacttacatgacagaaagaagcctgaaaagacatttagtccaaccactcacaggatacttgaacccctctgcaatctatcaaattgcatttaaatccttccagtgacagggagctcactacctcctgatgtaatacagtccagctctgagcagttctgttagaaaattctccctgacattatcctgaaatccggctaagaaagatatgaatttgtttctgcaatcatttatttgaccttctggattatccattatttactggggtggttaacagggagttggcatcatttacttcctactgtaatctgtgaggaatgtctctgttcctagacccctttgcccaagcagatgattgtttggtttgcccagggggacacctgtttgatccacaaagccctggcagcttcaccagcttgggacaaaggtggttctccctgaagccccagacttacccctttggccttatggtgaagtgtgtcttggaaagtcatcccgctgccccagtttttgatcctcacatgccgtggggaggacaatggggttgcatccagcttgaccagagattctggagacttctgcaaggggaaaaaacagggttttctctcaggtctctcctagaccccaggccctgccttgtcttagagtggggaaggatctatgggtggccacacagggcagggtgtccctccacgtccctcctctgtttccagcccccgggcgacctgggccagtgcctgctaagagctccaggtgcctgggccactggccagggtcctctcctcacccagggccttgccaaatgcttcctcagaggaactgctgtaggagggactaaagactaagttggaggtgagaaggatgactgtcagagccaatactactgagaacagaaaggcaccactgcattccttccctgggagcatgtaaggacatataagccaccctctctcccactcctacccatttctctgcccacctctctagaatggtatggaacccaggagatagaagctagctgtcagtgcaccttgggggccaccactcccgaccactgggggttgtctcactctcccaaagcccttcctcattcccacctgttccccccaacaccccgggtctgggactctccactctccatccaaaccctggcttcctgccctctccagcccagctctaacaggctgctatgtctgcatctgcctggaccagggaggaaggggcttcccacacccatgtgactcactgacctttcccgtctccacgaggggctgcggggactcattctgctgcttgctgaggttgtgatactgaaggtcatcctgtgtcactggactgtgaaaggaaacagctagcatgagatgcggtatccaagggtggaggggcatcttggagttccctctgccagtaacctcatttgacaaatagggaaactgagtccagagggtgggagtgaattgttcatggtcggtctgaccaactgcctccccatccccatcttccctcttcctcttatcttcttcctcccgcagagccaaaagggcatgagcagagctgagccaaagctactggcccaaacagcagcccatagaggatctggaaaatgctccttcaccctctggaaaagccaagtacaaacgtggcacaggggctgggtgggcagagaatggctccaacaactctgcctcccagactagcagcactgcccagcagccacttggattggttgactgattgactgattgatttatattttttgaaacagagccttgcactgtcatccaggctggagtgcagtggcatgatctcagctctctgcaacctccacttctcaggttcaagtgaacctcccacctcagcctcttgggtagctgggactacaggcgcacaccaccacacctggctaatttctgtatttttggtagagatggggtttcaccatgttgcccaggctggcctcaaactcctggcctcaagtgatcctcccaccttagcctcccaaagtgctgggattacaggcaggagccaccgcgcccagccccagccacttgtttttcatgacttaagctttatttaaaacacgtgatttatcttggtttatttacttattctacatttctattacagaaaatgttaagtgtacaaaagtagagagaatagtctaataaccttccatgggcccttcacccagattcaacccaattcaacagtgatcaatccacggccgatcttgtttcacctataccttccctgcccccatcattattttaaaacaaatctaagatgtcaacttaccttcaccaaggtaaaaacaagtattgtaaaaattagcattaaattacagattacttacaaattaaattaaacattacttaaagtaattttttaaattaaagattacttaaaaattcagtattctgaaaccggtcagaaaccgtttgtcctggtttgggatttcaggttcattccggcgaactgaatagtcaccactcacacatgcctcccctaggaagaatcatcccaccagggcaccttggtttgaaagcatattgtgttccatgttcttggcagtattttccagggtatgcatgcttgtgcatctgagggagaggtggttagagactattatgagggtctcacattttctacttgatatgtatctatgatgttgaaatgcgataaatgaccatgtattgcttttattatagaaaaaaatgactatatgttaccattatatcattaattatatacattacatagtagcagtaagtgattttttactataaagagatactgtcttaaaagaggatgtaagtatgtatcagaaagcaacttatatctgaatcaagggattccacaaccctgtaaagggccttccatgtgaccaaccattgccaactggcaaagatgctttgcttagccaaactttactcagacttctgaatcttctgcggggcccatccaagcatttccttgtaaaaatgcagttttagcaaaagaatccctacttagtcagtttaacaagaaccctccatccttgatagcttgtcgtcctccccatctgatcagggtcctcatcctccatcctcccccaggcgatgtctgatcaccttagcctgtcctaagcaagaatcctgttaggttgggtgaggcagaattccccctatgcctgaagtttttcttggtaattttccatccacagaccccaccctgatccttggctataaattcccacttgctcctgctgtatttggatttgggcccaatctctctcctccactgccagaccccactgcagtaggtccctgtacctattgagatggtcctaaataaagacttccatactgttctttaacaagtatcattgaatacttttttctttaacacacctggtctagactggtgtggcctgccagaaaaaaacaaaacaaaacaaaacaaaacaaaacaaaaatatatatatatatggcaatctggatattcagaacagataagccatggctgagcagtcaccatggggctgcttcaataggaagatgtatgggggatggggtccccaggggacaactgcccctcttgctcttttgccattccccctcaccccagcacccaccctgcacccattcttgcttcctctcacttacttctggaagtagagttggacaatttcagaagcttcagaccagaaacatctacagtactgtttagattgagaagcaatgtctttgtacatttaacataatcattttaccccatatattaaaaagtatcacttttaaaagttggctaaaaatccactctacagatgggccacgattagcctggtcccattgtccatggctggatgttagactgtgtctagttttgccctcatataaataatgccttaaactgtatacacagtctgatgctggctgtgtgtgtgtgtgtgcgtacaagcacagaaaaacacctcgagggaatgttagtaaatgttaacagtgactagcattaagtgataagatcataggcaatttaacttttcttatacttttccacatcttccaactttttacagtgacaatgaattcctttcataattggacaattctttaaatttgaaaaatgaataaacaaagttttgcccaagtcccctcccacaccatttcttctggctggctgctgggagacagggtgacagggtgaccagactccaggtgagcaccttcagaatctggacacttctttccaaattatcacattgttgattctgaccccgcagacgctcgtgcaatttgactaaacccacagctgtgcggggtcctgaggaccctcaaggacctgcattcaaagagagtgctggcagctgggcgcggtggctgacacctgtcatctcagcactttggaaggccgaggtgggcggatcactcgagcccaggagttcaagaccagcctgggcaacatggtgaaaccctatctctacaaaaaaaaaaaaaaaaaaaaaaaggtgccaagtgtgtggtgatgcgcgcctgtagtcccagctactcaggaggctaaggtgggaggatcacctgagccctggaggtggaggctacggtgaaccgagacggcgccactgtcgcctgggtgacagagtgagaccctatcaaaaaaaggggtcgggggcgtggggagtgctgtgagtgtggtggcttgcttagacccagaactttctacaagcccttgaccagccgagccggtcacagctgcagactgtggcttgcctgggctgacctgggggccagattcccctgcggttccgacgcctccctgcctgtttacctcgccttacaccaagagtggtggtggggacagatggggagggtgggggggatggcgccctgggaaatggatctcatttgtgaaacaagaacctccttgctctcttaaccatatttgatccaaagagtgtagcaactttcaaccccagtgctgaaaaatttcaaaagctcccaagtgctgaaacattcgcagcgcaactgaacccgcatagacgcctctcatagtgcatggggggtttctctgggccgcctgggctcctgaattgcgacctctcaagcgttctaaaagaggaaaacgcttttagtaaaccctcaggaatcccacagagcgcacatccccgccctccgatttctcccagagagagatggggccccgcccgggctcggagcccaccgcttccctcccagggcggacttcggcccgcggggtacctggctctgcgcgggcagcagtcaggcttcggccggaggatcctgcagagcagcctgcacagccggggcccggcggcctcgcacgccgcgcccagcccctcgcgcatggcccggctccttaggcgcagctccgctggctccacgcgcctcccagcgcccgcgctttatcgctcggagcctgcagcccccgccccgggggcggggcgagccgagcagcggcgggggctcttcacccacgcgatgtcccgggccgagctgcccctccttccctgccttccccggcgatcccgggacccaagcacccaggggcccgcagccggcgtctgccgcggagtttggatggcactggactgtgtctgggctaagatgcccgggtcgtgttgggcctcgtgtttgtttattccccataaaatcgcaataaagcttatgataattaccaccattgcgcggtgctttctagtttacagagcaaatttcctttctgtcatttcgtccagtctcgttaagcagacagctctacgttgtcatctccactttattgaggcattagctggggccagagagattaaggatcttgtccagggccacacagcagccccaggaccacagcccagcctaagctcacaccacctcacaaggctgtcccgggaagtgttatattacttgttacatacagcatgttctgtgcgattccagaaaagaaaccaaagaaagcactgggttaggggtgacttctagtaggaatattgcaggagggatcgtaagccccttccggtgccttgcaggccccttgtccttgctacctggactgcgcagagcctcctgactttgctccgcctgtaaggtgctccttatctcaagcaagataacaggtagggtccagacccagatggggcagcagccctggacctcaacatgcagggggcccttctggcagtatgcaagcacattaaatcccgaaccccagttaaagcagaatgaaagagggcacatatttatttctagccaactgcagcccagcaagcaacacatagctcgacttggttctgaattccatgtgtcttcacctagatgctcccacatttgcctttgtccctaaaatacacacatgcgtacacacacacgcgcacacacacacacacacacacacacacacacaccatgctcatttcctaattgtttacgaaggtatagaatcaccagtcacttagaataggctcttggtttcatgacacctgaacagattagaaaattactttttaaaatcaaaatatattcattggctctcattgctttcaggatgaaatcccctttgcacggctgcttcctcttccagtaacatcacccctccctcacctgagtggatgttgcactctgggtggacccacctcctccccccaactcctccccacactacagcctgcccaggcacctctgcagtctccccagcagaggcctctagcacggtcccagtacacccacaactacgacctcctgtctacactccatcccacctactcctgacacaatttctccaactcgcattcttcatgtacgtctcccaaaccagcgaggaagacatccgtttccccgtttccttcctgacagcaggaactgggtcatttttctctgtctcttgaacagtgcagcacacagtaggccctctgggagagcacagatagtgtagatgttatgagtttggacttgactcaaaccttagctccactattttccaggaattgacctccagtaagtcactcaatcttgtaaaacaaggataatagttcctgtctcattattgacacagtactaaggaccttataaatgttatctattgtttttagcagtggtgtatgtaagtaataagtggctcaaacaattcactgatgagcagaagggtttcattgatatcttaactcccagcacagagccaggggagtgggcatctgcatgtactcgtcaatggaatgtgacatgcccaggacacaaaggtggcagccccatgcaggggtccctctggactgcctggatctgaatcccagctctgtcatttgctaattatgagatcttgaaaattactttaactttctgggcttcagggtcctcatctttaaaatgggataactaaaattccttccttgtggatcgttgggagagttgaatgaattgatgtgtgatatttttttcttcatcccctcttctatctgactggatcctttccgccagcccaccccagcatgaagaggagggggaaaaacatggtcaagttagctggtttgtcacatgtccactgcagctcagggccctccctggagtctgaactcatatttcctgaatggtccaaagacacaagtcagcccccaaagacatagggataactggcaatgctattagagggtgtggggatgagggttaaaggctgcagccaacaccgtaagggttttgggagattcagggtgatccggaaagacagcagatagatcactagggcccagagttcgagggcagcctggacaacagagcaagattctcagctttaaaagagagagagaaagagagaaaagaaaaggaaagaaaaaagaagcctaggtgcggtggctcacacctgtaatagcactttgggaggccaaggcaggagaattgcttgaggtcaggggttcgagaccagcctggccaatatggtgaaacaccgtctctactaaaaatacaaaaattaaccaggcgtggtggcacatgcctgtagtcccagctactcaggaggctgaggtgggagaatcgcttgaacctggcaggtggaggttgcagtgcagtgagctgaaatggagccactacactgaagtctgggcaacagagtgagaccctgtctaaaaataataataataaaaaataaaaatagaaaagaaaaaaataggggatgagttgggtctggatgagctgagggaggaatgtgctatgcacaggcactggggagctgaggaaggctgggatgggcaggtgggatgggctctccatttgggtagggtgctcgctcagaactctgtggaagctttaatgtggtgacaggcaatggggagccactgcaggctctagaataggggcgtgacttgctagaagtccagttggcaagagagagctcagagccagtgtggggagctggagtcagggaggtcagctggcagcggctgcaatgatgagatgaagagaaggaaggggcgaagccactgcccctctacagcccttagaatctggacatctagaggccatcaactctgtgttctcacctcttggtctcttagccacctcagttgccatagcccagactgcctggtttcaaagtcctctcctccaattcacctaccacaccacagctagactgtcctgctcaagaacctacaatggctccctactgccagggagagcgagtccaaatctcctaccctgcattcaagcccttgtgctctggcctggctttgcctatcccagcccccagcccactcctggccaagcacagtcaggctcatttctgcctctgagcctctgctcacctctcccctcttcctgaatgtccttgctactcctctgcacctgtctacattctacccatacttcaaaggccacagagtccacctgacttctgtgaagcccctagagggaactctgttcaattttgctccatcacctcctaactgccaacaccaccgctaatgatagcaatgccaacagtgccatttgctgagcacttactccaggccaggccctgtgctaagttgctcacatgggaaagctcatttactccttcctccaactccaggaaatcaccattgttaccctcttcttatagaggaggaaattgaggctcagaggctaggggacttgccaaaggttacagggctactgagtggcaaagccacttgctcatccccaagcccatcctcttagccactggcattttttgccgcctctgacagatacactcagggccgtcatgctgtacacatccagggggcgccctaccctttgtagtccatgggaaaggctcctctggggcggtggggttgtgtggctatgtggtggtcttgtgtagatgggggctttggtttcagttgcactattgcgttattgcagattgctttgtctttccacctgagcgagcctcaagcctgtcccttgacttgttcattaactgactcattctcttggttctgccctgggaggaaggatgggcccggggaggagggacatgaagaatccaaatctgggctcaagttccaactctgtaaactctgtaattaccggtcattcaggctgggggaagtcataatcctattagagcttctatttccttatctgtgaaatgagtcaatcatatgaacttcaaagagccgccacagagatcagttgagatgcaaagtctgtcaagtggcctgtgtagctctggcaagggcaggtactcagtagatggtgatgaaccctgatgtggactaagtgtctgttctgtgtgggcacagtgctgagtgctgtagatctcatttcatcctcagcacactatggtgagggcccccttgggttcctcataccacaggtgagcactaagtttcagagaggttaacttaccctgcgtcacacagctggctagaggcagcacccggcaactagtgcggccagcacagggcttggaactagtttgtctaacatcctgatgcccttcaccaggatgatattcaaagcatctagcaagcggtgaattggaatggacgcaggccaggatgctgaccctccacccccacccctccgccatggtgcccaccacgaggctctactgcctcccattctcaggccaagctccttcccatattcttccccaaagattccaagcttcttcagggccagccccgcaccatgtcccttccttcttcacataccgtcaggcacagcgaggtgcccaataatcagcggctgcattgaattccaacagggactttcaagaagcaatgaggagatcagacctcaggtgagagaggaggagcagagagaggaattctgagtcatcggtgccgacagggcatgggtgcccaaggagacaagcaggaggttcccccagggaaaactcacctggaggtggcacagggggctttctccacattgttgttgatgtctttttccccattcattgcatactggtggaatttggtcttgaacagaaatttccaaggacaggccatctctatggctttacaaagcaggtcacttatgtcacttatctggatttgagctcagatgttcttcactgtggggctgaagaagggaagcagaggtgagggaaggttgaagaagagtttacagaacagggcttctcagtggggttggctcactggggaatcgatatgaacgccagcatgagccccttcatcaatgctttgcaacctctgtctctatttcggggttggaatggggagccttcggacacagacttcaattctgtaaagccacctaataatcttaaggaaaaacaatgaacccccccatccccatcgccacttaacaaggacaaactgtatgcaccatgagcctccttagcgggacctgctggcccaccagacccagccaggcttcatcactggccacaggcagggtgaggaagagcccggaggtcgttgaaacaggggtgcctttgcctgaccctgaaagacacttcccagcagctctcattatcaactgtagttttaggtctattactggggaaagcactcaagctgtagctacctttcttttaaagattttgcagttgttgttctaactcagcattattacttgctcctcttttattatgtggggaggaaatatttgtgaaaaatgcactcatccctcgaaatctgagatcatctcctttgaaacagacaagaggaagatcaaagaggcaaaaactctgtctttagaagacagtgtagtgaatatagtccatgtaagaattttaggccaagtgcagtggctcacgcctgtaatcccagcatttcgggagaccgaggcaggaggatcgcttgagtccaagagttcgagttcagcctgggcaacatagggagaccttgactacaaaaaattgaaaaattagccaggcaggcatggtgtcttgtgcctgtagtcccagctactcgggaggctgaggcagaaggattgcttgaggctgggcagtcaaggctgtttccaaaaaaaaaaaagaaagaaagggaagggaagggaggggaagggaggaaaggacaggaaggaaaggaaggaaagagggaaagaaagaaaagaacttatagccccatagacctgggttcaaatcctggaatcctagctctaccagttatgtgtcctggatcaagttatttaactgaaactcatttattcatccataaaatgggtttaatatatccatcttaccaggatttgtggggaggttacatgttataaggactgataagataatctctgttaaaaaaaattcttagcagatacaaactcccattttgaacttttcagaaagtttttccttgtcatgtaacaaccacagctaatatttgcagaaaagaaatctgaccaagatgccatgttcaaatgaccaaacctcatgtcggcatttttttacctagactttccaacaccttctctctgtaggcagaaaattcccacaattttctgagccttctcaaagaggaagaaacaaaaccctctcaaaacagccgcttccccagaaatgaaggcaactcaccttgcagctggctgcactgcctccccggggtaaggacagtcaaaccaggaagagacctgtgccttgagaacttcgggactgtctagaactgcccagtcccctcatcaaaggtggccgagagattttaaagcaggaatgaggctgagttctctgcggccggagcctcagttttcgactcgctacaaagttatgaacacactggcagccaagaagtatttatactcaccccatgccatccagagagttgtttttgcataaaggtctcttttttttttttttttttttggtttccaaagggagtgtccccagcttgtgtacagttagctaatttatgactgtgactgccagggcctgtgggtgacctgatcttgctgttacatcactctgtgtggtgcctcttcacccaacccacctctttctggtcccaagtccaaggtggcagttcatcagcagggtggctgctaagatagaggcaccacggagccaggttttatttggccaagccacatggcctcactttcagcatctgggagattttttcctcagcagggagctctctggttaatccctgtccatcctctcacttttccagcttgtgacaggagtccaggaagctgggggaatattgacagtatggaacacactggaaaatgcccaggttccagaaggccagtgatattagatgatgccattattccagacaggcctctgaactagtcacttgaggtttttggaagggaaaggaaggaaggctattggcacagagaggcctctgagatgttggtcttactaacacctcatcctttagcactaggtaagctttggtggaatggcaggtaggattaaattccttcccttataatggaaatgatcacttcttacatgaaaaagtggtaatttcttataatttcatttcatttattttatttatttatttatttttgagatggagtctcattctgtcaccaagactggagcggagtggcacgatctctgctcattgcaacttctgcctcccaggctcaagcgatcctcccacctcagcctcctgagtagctgggactacaggcatgcaccaccacacccagctaatatttttgtgtttttggtagagactgggtttcaccatgttgtccatgctggtctcgaactctgaagctcaagtgatctgcccacttcagcctctcaaagtgctaggattacaagggttagccaccatgcccggcctataatttcattccttataatgcctgaaatacagtattcccatacatatgcatttttccatcataaaagtaaaataacaactacattagggagaagttgagaagcaaagggaaaaaaagtcacccttgatctcaccgtcccaacactttgctaccactgtccactcatttccttcttgtctttcccaagttaaattcagcataatatattaactttgtcaaagataaactaagagccacaaaaaagacatggatatcagagctcaaggatctttgttgagtgaatgtgtggatgaatgaatgaaaaataggagtaaactggaatgagttttaacatatcaaagccatcttcccttcaggcttcagctcaatgctgcggctcaatgctgcggcttagagctctggcttatggagcatctgccacgaagagcaatgcattttagaaagtgacaagatggccgggcgtggtggttcatgccggttcagttcaggtaaacagcactcagggaaagagtcagaatcctggccttagggagtgcaaggcctagaaaaataaatggataattatagtacagagtgatatctgtagtgatggagggatggtatggtgctgatgtagctggttctgccttgttcaaggaaagtttacaagggagaggacaccagggcagggttttgagggatgattaggagtttgctacactctattccctgtagctgctttcacatattctggctatacctgcagcaaacacatgtattaacacatttaatgcccccacccaagagcctctgagggggatactactatttgctattcatggaatcctcaagaatcccatctacttggcctcaaagcacttcgttgattttccttcagaaaatgcagttattcagttatgtgatctgtcaaacatgtcacctcaggtgcccctctaacacagcgtctgcttggtgtggcattcttgacctgtggcagggtttgagtccaagcaccaggcttgaggacacatttggaattagccagctgggaaggaaatataatttctcctcaatcatcttaagttatttgttgggaaagacactgtaacaaaagacagattaataatagagaaaaaaaagcaagtttgttaatgtctgtgatgcacaccacgcaggagaagcctcagtgaaagtaactcaatgctgcggcttagaactctggcttatggagcatctgccatgaagagcaatgcattttagaaagtgacaagatggctgggcacggtggttcatgcctggaatcccagcaatttgggaggctgaggtgggcagattgcttgaggccaggagttcgagaccagcctgggcaacatagtgagatccaatctctacagaaaaattgaaaataaattagctgggtatggtggcatgcccagctactgtagtcctagctactcaggaggatcaggtgggaggatcaattgagtctgggagttagaggctgcagagagctatggtcgcgccactgcaccctagcctgtctcagaaaaaaaaggaaaaaaaagagggaaaggaggaaaggaaaggaaaggaaaggaaaggaaaggaaaggaaaggaaaggaaaggaaaggaaaggaaaagaaaggaaaggaaagagagaaagaaagaaagaagagaaaggtgacaagacgaatgcagttgtaggcttccaggggtggcaagctgtgagaaggtaaacatgtggaagtgagctaaggagtaggatagcaaggtttgtttgtatttgtagatggctctggtgccatctctgagctgataaaagttatcttctcatcttgatgctgggaaagcatctttctccaaggaatgtttatggcttgctgcaggcaggacagggcagttcagatagccctctctctagctgctctttctcaaatgccttcagctggagataatcaggatgccatatcggcatatctgggcgtggcatgttctggtttcctttagcctgaaaggtcccaggttcgaggcacagcttcactgctgacctaccatgtgatcttggaaaagccacttctctctgggcctgacttttctcgtgacaaaaggtgataaaaataaaagtaaataaacgagatacaagaaaatgctttgagccattgagcaaaaagtcagccaagtaagaatttattatccttgctgggagtcagagagtaacaatttcccttttgacaaagagcaagtggatgccaggacttgccttctgaagtatccttcctcagacaggtccccgtcccaccacaaacacctgcttttaaattcccggtcctctctctccctttgtaacattaaattcatgtatgtatttttaatttttaattggaataaatttcacataacataaaattaccaattaactattttaaagtgtaaaatgcaatggtttttagaatattcacaatgttgtataaccatgaccactatcccaatcctctcttaaccggcaaataggaagaggaggagagtaaaaatagtgataatgattgtgatgatacacagaggttaggagactcaccaaggtcacacagcaaacagccttcaagccgggacaaacagtgagcttctggtaccctccccagagcgcctgtctgcagggtccagggctggccccctcctggaggttggcaagtctcctacggcttcctgggcctgcacggctgccttcttcttggccctgcccacccagcaggccgcattcctggcctcttctgatgatggatgcagcaggtgaattctgctggggagaaatcaaagagcaccttgaagggcaggcgggggacaggtgggccccccggagggtggcgatagaggttccgaggctgctgtggagaaatgccctcctccctgccttcccaggctgtcctaggcaccatgctgacagcaatcagcacgggaatgtcttgggctcattatcctggaattgattttccactggattgatttgatctgtttctatgccaggctgtgaagatggttcagatccagttagccctcggaagttagcaactttgaaggcttttctacaaaaacaattgatcagagctaacgtttactgagcaaggcacttaaacataccaagcatttaatccacacaacagtcctatggggtgggaatgatcactagccccattttatagatggggaaattgaggctaagcaaagccaaatgacttgtccaagaccacacagtcaggtggtggcagggctacaaccctggagtcccagcttgcacttgctctggtgcttgctccataaatacagccaggccctgggctttccatttattatctcagttaatcctccaacaacgttgtgtggcaggtactattataatactatccccaattcaccaataaggaaaccgagacacagaaagtttgagcgaccagctcaatgtcactcagtttgtaagtggcaaagctgaggactgacccaggtctgagaccttgactggaccccacttgtgcgcccactgtgtgcttacccattttgctacaggtgagtacacccaggggcaaggagaggtgacaatgagagagtgggaaactgagatcaaggtaggagctgaggattacctggggttgaatgttctcccctccatgtcagtgtctggtgccagctctgtgcaaggccatggaaggaggagacggagttcctactgtgtgccccgcctcacagaaagtgataaaaattttattttagaaatatatttaaggtttaaagagagccagtttaaggtaaaatgtttaagcaaataacagtataggtgcatgcagatgtgacacaaatgataaaggtgggacgtgaaggaaacacaggggcgtggctcttactctctatttgtgtgtgtgcatgtcccgtggcgagtcacgtgtcctgtggggccttcactccactcagctgtcaaaggaaggcattgagaagaggcattcctgggaccctcccggttctgaggccctaacaagctcaagtccccagaccgtgagccctatgtcatttgtcaagtgaccactggagggcgtgagtcacacaaattccagcagctccgaagctgaggtcttccccggtttccaccaccttgcctgactcggagatgacggaagtgaagtgaagggatttttactggaaacgcccggtggtcactccaggatggacaggcccagggtggaccaggaaggtggacaagctttgctgagtccctggagtgtgactcaagctggctggtgcaggcctggcactttgctgctgtttcttttaaaagcctcgtttcttgtcaagcctccccagaacaccctcccttccctgccctcccgttttgcttcctgggtgcccgtccttctaggcggagaaacaacaaacagagtggcacctggggtagtcccagggtgtgatggttcaaggggcaagattcaggggcaagtagtgtatctgggaagtgcaggggacatcaggaggggaatgaggaagtgagacagagaaagggaggcagctggtaaaagtactatagggcagctttcacgatttgcaaccagactcagccccaccagggaacttgaaaaatggtgcagaaaacagggcttcaaatgatcccatccaaggggccaggggctgggctactgacccagcagttccagagcatcactcattgaggctgctcccagggaggttaaattcccaaggcctgctgtgggcagagcagccttccttggttctgggaaagccctctagaattcacaaagtgcactggaatgagacgaagacagacgtcactgctacagggggtttagaattgggtcaaatggaggaagagaggtacctcctccccaggtacagcctctgagggtgctggatgctgatccaagaagttctaaatgccaagagcttcagctaagcccaccctcttgtatccttccctaagaagcgctgcatctttggttaggccacataagccctccgggcctccctttcctcagttaagggtctctgaggcctttccagctgtggtatctgggctttctgagtctccccacctcatacatatctggtctttgatgctcttggaaaagttatcttccccaccagagtgcccctgaaaggcagaatgtagcaacgactggcaacttctgcagagcctgaccactgtgtgcacagagatgtctggagcatcgctctctacgagagccagaaacaacccaagtgatcagcatagagaaaggcacaccaggccagctttatagtgtaccattacgtggccaaaaaaaaagtgtattaagaattttttggccaggtgtggtagctcatgcctgtaaattctagcattttgggaggctaagatgggaggattgcttgagcccaggagtttgagaccagccttgccaacatggcaaaaccctgtctgtacaaaaaatacaaaaattacaaaaattagccaagtgtggtggtgaacacctgcagtcccagctactcgagaggctggggtgggaggatcgcttgagaccaggaggtcgatgctgcagtgagctgtgattgtaccattgcaccccagcctgggcaacaaagtgagaccctgtttcaaaaaaaaggatttatttagctgggcatcgtgacaagcacctgtagacctagctactaactgaggtgggaggatcacttgagctgaggaattaagagaccgcagtgagctatgattgcaccattgcactctccagcctaggcaacagtgagactctgatctcttaaaaaaaaaaaaaaagagtctttatgataagaaataatgttcatgatatcagtagaaaaatgataacccctaacatgtaaaataaagacagaaaaaatgtgagaaaaaaatactaaaatattaaaaagtggtagaattatgggcaatagttatcttttctttacccttctctgtatttgctatctgtgcatatgttagttttataatataaaaagtgactccttactttttgaaatactatctcagacgtgtggctgtgccagtaattcacctttcccaaaggaaaagagcctctagcacccattttcattacaggaggcacagagagggcggctgcccacgcccctggctgttctgcaggagttacaccggttttctgagcatcgtccgtgagttttccaagctgggccctggggatccagtggtgagtgatggcagacaagctggctgtctctctccctccccactctgctgccaatagagatggcaagatgagtcccagagcctgttggaggcggtgcatttgggggacaggggctgcctccttaaaaacagagtggaaaccagggggacaggccaagtgcatcgaaatgctaggacggttggccaaagctgagcggctcactcttgaaacagaggtggggtctggctcttggtgggtgaaggaagaattggaggaagggaggaagggtaattcctccagcaatttgagtgggaggctgtggggggcaagctattaataacggcagaggccactgtcaggcagactcagggacaagtctcaggcctgccacctaaattcatctgtgggcattggagaggttacttttggtttcagtttgcttatctgtaaagtgtggataatcacagagtccccacctcctcaagccttggggaggagtagcgagaggatgtacgcagcacctgccctgcagttcagcttgagcacgctcctatcttgccagagccggagcgtagctgtagctttagcagagcagcagcaccgagctggctggctctgcctactgctattctcccagcacctggcacatgctggagctcaagaaacaatgcccagctgactgccagggcctttttaatgtgttatctcatgcattccacaccaccacactgtgaggtgagctgtgtcaaccctattttacagatgacaaaattgaggcttaggaagattaaagaatttttcccaagatcatacagctaaagctgcagaaccaagattcaggtccatttgattccaaatgcttctgggaggagcatcttagggacaccaccggtctgaggctcaaggcagccctctctcacccaagaccaagctcttgtagacaacaactgtttaaagggcaaagacagctcctggggaagaggcaggaaaatcaatgaaatgatctgtgatgctgtcagggccctggaggaaggactgagcaatgcaggagttccccaggggctcccgggagggcccaagaggccaggtgggggcggggttgggcagggaagcctgggggagtactctcagaggaggtgacagtccccaacaggaggaaaggctgtcctcctgctgagcttacaagggaagctcccatgccggaaggaagaaccttgaacttctgcttccaatacctgtgtctagcctgtctcatcctggcctgcatttcagagctccccccaactccaggagctctctagtaagccatgcctttgcacacactgttccctgttgctggaatgtccttcccgacagctccctagcccacccttgccctgactcgaatccccttcagactctgctttacttcatggcattgttgcctctaggggctcccctgaccctccatcctcaagccccttggggttccccaaatcccttaggctcccctctcctcaacactgatcccactgttttaattgtggtctcactgctctgtctcttttactagacacaagcttcccaaggccaaggtcatgtctcacatctctgcagcaccagcagcatgcttggcccagactaggtgctcagtggattttgttttgttgagataaaaagtcacgtgccattaaattcccccatttaaagagtataattcagtggtttttagtatattcacaaagttatgaaactatcaccactatctaattccagaacgtttttgtcaccccaaatagaaaccctgtacccattaacagtcactccctatttcttacccccaactcttagaaaccactaaatctacttttttttcactatagatttgcttattttggatgttttatacaaatggaatcatataatatttggccctttgtgtctagcttgtttcacttataatgtttttggggttcattcacgtggcatgtttaagttcttcattctttttatggcttagtaatattccactgtatggatataccacattttatgcatccattcatcaattgatggccatttgggttgtttctattttttggtcactgtgaatattgctgtccaaatataaatgtttgagtacaggtttttgtgtgaacatgtactttcaattatcttggaatagaattgttgggtcatagggaaactttatgttcaacttttttggggtactgccagactattttctgaagtggctgctgcaccattttatatcactaccagcataggaagagggtcccaatcagtccacatccttgacaacaccagtaatgggctttcttttttgtgtcatatagcttctagtggatgtgaagtagccaatggaccattgacgaatgagtgagtgacaaacggatggatggatggatggatggatggatggatggatggacatatgggagtagatacctgaccctgagttctgagctctgattttggtgggacagcctgcttccctgaggacctgcatcaagtagagacatagcaacctgcagcctgcccctgaccatcgtggttccagatgtccaatctgccttcgtcctcggtggctcttaagcacacaggtgacctggattcccctaagcctgtggaaggccctggagggggccccattctgggtgacacaagttttattactcatcagtttcagtttcttcttctgtgaaatggaaggagaaagagaaaagtcacacaagtttctgcagagggtgtggcacatagcaggtccacaataaataggagtttcgtttttccagtctgtttctcatccagtgtgtatataaatgccactccactaattatctactggaaaagagcttgagaagtggggcagtggaaaggaggggggacggggaaagccaggcaagagatttacatatggcaggaagctgcagcaggtcatcgagggagtgggagtcacagagcaaccctgggaggattgtcacaggcttcggaagaggatatcctcccagccatttgcatacccaagcaccactcttactgtcaggaagacctgagcctcctatttaggcctttggatgggtgtggagagagggcagcatctaactagtgctgatctgagatcaactgcaggtccccaggcaaaccccacagaagggtgaatcagattccaaggccagtgaattttatccattgtgccaaccttcatagacaaggaatggctgctcagagccttgcagtgagatcaatgttaaagttctgtagttttaaggctcagtaggaaaaagtagccacccgcatctgccctgggcagatgaccaatgacctgtatttactgcccctgttctagaccagtacaagattagctgtctgttgggtagtttccatgtaaatagtttgttacctggggcagttgaaaggtcaggaggaaaagaagaggggattaaaataaattgctggaacgggggactggcggcggcagtggtgctcacttgaggctccagcctgcagtgagcattgagcacttaatttatggagtgctcagctgaggttgccactagctgcttacgagaggccagcagcgccgtgagccaggcccacttagggaagattcttggctcatccatgtgactcaccttcctcatacgtaacatggggacaatggggatatatgtcacagggctgctgtttatcttgccggatgatctaatgcacatggatgaagaagggaatgtgggaaatacatgctctcagtcttaacagaagggaaaactgaagcccgcagtgggttggaagaggtggagaaaatgcaagaaacacagacagctttgtgattgcaactttgttccaggtcctggttgtgtctttaattccatattaattgttttgtctttagtacttaactcctatctggaagaactcctcattagcccatctgccctggcatggcccacttctgaggggtttccttggcctgtttccctgatgtgtatcatgtgacccctccagttgcctggacaagggtgggcatgtcaccagggagcagccagtggcctcagaggtaccctgatggacctcaggccctgcaggtttgaactggagacagaagagtggtgattgatagcagagttggtatccaaaagccccccagagagatgacggatggagctgcaggcagaagaagcctgcagtggggagtacagggcaggttgctgcaggaggagcagggagtgaggggttgagctccagaccaggacgccacaggcaggcagggtggagagaacccaggccctggagagaactgccttcagggtcccaaagtctccccaggctgcatgattcccgccatgtggcccagcttccttattgcccagagttttaccgtaacctcttgctctctggaagaacctgttcagggaacttgcatttttgcagctccatgacagagtctggcacaggtactattggttaattgaggaagtctgctattttatggcacaaatgagagccagcagtttgcaggcattgaggaaggagaggcgtatggactatgataagggacgaccctggaaagcaccctgatacggcattggctagggttctgaggaagctaataactttgccgaattttgttggaaaggcaatcaacctttttacagccctattaatgactttatcaaatcactgcttttatgatctcttggtgaacccatcaatccacgtagaacatgaagactcttgggttttctctccatgaggcgagagaggttgaaccactcgcataacgttatgtggcagttactgtctaactcagggaatccttcaggaagatcctcagctcccctgctaccttccacatacccaagctgctgtctgtctgaatatgcttttccccatgtaactcagagtgtcgggaggatgggggaggattcccagaacaagtggcacctaaacagggtttggtttgtttttgtttttgttttaagacagcgtctcactctgttgcccaggctggagtacagtagtgtggtcacagctcactgcagcctcgacctcctgggctcaagtgatcctcctgcctcggcctcctgagtagctaggactacaggtgcatgccaaaacctggctaactttttttttttttttggtagagatgggatctagatttgttgcccaggatgatctcaaactcctgggctcaagagatccacctacctcagcatcccaaagtgctgggattacaagtgtgagccacggcacctggcctgagcatctttttacatgcttgtaaaaagcatgtaaaatgtctttatcttcattggtgaagggtctattcaaacctttgctcaatctttaaatttgtttttgtttttgttttgagaaggagcctcgctctgtcacctaggctggattgcagtggcacaatcttggctcactgcaacctctgcctcccagattcaagcgattctcctgcctccgcctcctgagtagctgagactacaggtgcctgccaccatgcctggctactttttgtatttttaatagagacagggttttgccatgttggccaggctggtctcaaactcctgacctcaagtgatccacccgcctcgggctcccaaagtgctaggattctaccatgcctggccccggtctttaaatgtgttatttattttcttactgttgagttttgtggattctttctgtagtctgggtacaaatcttttgttggatatggggtttgcaaatattttctcccagtctttgcagtttcttaaagtgtatcttgcaaagcaaaagtgttaaattttattgaactacaacttgtcaatgttgttttgtgtggatcactgcacaagggtttgaagaacgtattcactgagctgacaaggaaagaaagggagttctaggcaaagcaaacagcacatgcaaaggaatggaggcctgaaaaagtgggctggggttggggaattctaagtacagttgaccctcgaacaatgtggggggttagaggaactgacccctgcacagtctgaaatccgtgtataacatttgattccccaaaaactttactaatagtctactattgaccagaaatcttaccgataacatagttaattaacacatatttttatatgtattatatactgtgttcttacaataaagtaagctagagaagaaaagttattaaggcaatcataagtggccgggcagggtggcttatacctgtaatcccaatactttgggaagttgaggcaagaggatcacttaaggtcagaagtttgagactagcctgggcaacaaagtgagaccccatctttacaaaaaaaatttttttttaattaaccgagcacaatggtggatgccaagtagctagtcccagctacttgggagactgaggtgggaggatcacttgagcccaggagttcaagagtgcagtgagctatgattgcaccactgtactccagcctgggcaacagagcaagaccctgtctcaaaaaatatatatttttttatatattagaagggaaaatatatttacaattcattaagtggaagtggatcacaattaaggtcttcatcctcatcatcttcaggttgagtaggttgaggaagaggagtggttggttttgctgtctcctctcagggatggcagaggcagaagaaaatccacttataagtgggcccatgcagttcaaacccctgttgttcaagagtcaactgtagctcaatattgttgaactacaatattgtacacttaatattggtacactacaaaattggtatactcctcagtaccaattttctgtattagttgatatgcattgttataaaggaatacatgagactgggtaatttgcaaagaaaagaggtttatttggctcacattttgcaggctgtacacaaagcatagtgttggcatctgcttctggtgagggcctcaggaagcttccaatcatggcagaaggcaaatgggagccagcacatcacatggtgacagggagcaagagagagatgggtgtcggagggcagtcctcaactctttttaaaaaccagatcttgcataaactcaagagtgagaactcactcattaacatgaggatagcctcaagccatttatgaggaatccgccccatgacccaaatacctccccaccagggcccacctccaacattggggatcacatttcagcatgagatttggaggggacactatgattctagtgtgtgcatccatgtgtgtatctgtgcatgtgtgtggctgggaggggaagggaaatgactggatcatagaagggaccagatcataggctcttgggggctgtgccaaggagtgtgggcccctcatcctataggtaagagagctaccaaagggtacggcaattgctcacttttatttttgttaacttttttattataagaattatatgtggctgggcacagtggctcatgcctgtaatcccagcactttgggaggctgaggagagtggatcacttgaccccaggatttcgagaccagcttgagaaatatggtgaaaccccgtctctacagggcaaaatgctggttaaaaaattagcaggtgtggtggtgcatgcctatagtgccagctactcaggaggctgaggtgggcggaacacttgagcccgggaagttgaggctgtggtgagccaaaatcacaccactgcactccagcccagatgacaggagtgaaactctttctccaaaaaaaaaaaaaattatatataatttattgtagaaaaatatagatcaatgaaaatatgattaaaactctatatacttctaccacccagagggtagttacactattaccattttggctggttcttctgttcacacacaaacacacacacacacacacgtaatttttctataaaaatgagatcactccatatacactgttttgtaatgtggtattgtttttgcttaataaataatatatgtgactggttttccatgccattattcttttactgtatcattttttgttaagataaagttttttcctttattataatttaatgtaatatttcatgcattcaaattaaagcattacattacaaatgtaatcttatgaattaccatggactcccatctggctacaggatgagaaaaacctactcccatcatctctgtaggctagcatatatttagttcttattatgcgctagatactgttctttatgtgcagtaactcctacagccatcgaaaaaattctctgagatagtgatattattatcaccatgttactatggggaaaggcacagagaggttcatcagcttacccaaggtcacacaggggatgagtggaagaagtagggctagaaatgttggagatggacttgaagatgaatttatatggaagataagggagcagaggtgacagcagtgacttctgggtttctggctgttgagactggggggcagggatgcccctgactgagataggaaaggcaggccgaggaactaatttaggacaaggataaaatcaggaagtctattttggaggcgttaatgttaaaggacctgtgggacatgaaaatggaaatgaacaaatgactatagaatgaacaagtgcatgaatgaattgatgaaagacaaatcctcaatgatatggtttggatctgtgtcctcacccaaatctcatgtcaaattgtcaccttcagtgttggaggtgggcctggtgggaagtgattggatggtgggggtggatttccctgttggtctgttttcgtaatagtgagtgagttctcatgagatccagctgttttaaaagtgtgcagcacctcccccttctccgtcagtcctgctcctgccatgtaagatgcctgctttgccttctgcaatgaataaaagcttcctgaagcctccccagaagcaaatgccagcatcatggttcctgtacagcctgcagaactgtgagccaattaaacctcttttctttataaattacctagtcccaggtatttccttcttttcttttctttttttggcaaggtcttgctctgttgcccaggctggagtgcagtggtgcaatcttggctcactgcaacctccgcctcccgggttcaagcgattctcctgcctcagcctcctgagtagctgcgactacaggtgcctgccatcacaccggctaatttttgtatttttagtaaagtctgggtttcgctatattagtcaggctggttttgaactcctgacctcaggtaatccacttgccttggcgtcccaaagtgttgggattacaggcatgagccaccgcacccggcctagtctcaggtatttctttatagaaatgtgagcatggactaatcaggcaagattattctcagcacagcaacagacagatctggcccatcatgttaggaaaccataagttgaagtgtaatgatgggaaggttcacagctgcttacaacaggccagggcagagaggatgcatagacacacaggatgggggcaagttgttttcagtgatgtttatcaagtacccaggatccagcagctgctccatctgggagtggctgctgctttctctcctgggaagattccttctgatcttgggagtaggatttggggtgaaggctgtgtccagaactgggtcctgccaccccaacaccctgctgagaacaaggcacattccttagcactcagggtaggagagaggcggagggatggagaggcttcaatcccccttctcgggagacaaagggctcttcatgatttaattaaaatgcccatcaaaacctcagaagataggggttagttgagatgaaataaaaatagttggagtgaacacattacaatttcatttcatttcagggatatctatgaggttctaatattagagagagacttaatctatcactaaattcctatctggcctgccacactgggggcatttctgaagatgtccaaattctgttttgagaggcctggagagtcctccgtcctttgtttctctgttcgaggagaaccacagagtgtgacagtgtttggtatgtggcaggcactcaatcaatatgtgcagaaggaaggagggagtttcttctttactcctttaagcttcagatttagaatttgcctaatcttggggtgagacaaagccttgagacctccacagaggtttcctttgcatggacaacttttcctccagataacacatgactcacttccctgcacttccagtctttgctcatgtgtcacctcctcactgaggccagccttgccacccctatttaaaatgacagccctccttcccccaaccctcctgacccccccaccttgctctgtgcatctcccccagagctcaacctcacaccttacatcatttacttactatttgttcattgtttctcctcactagaatgaaggccccacaaaagcagggatttttgtctgttttgcttatggatgtataagtgctatgcatagtgcctgcctcaataaatatctgttggacaaatgcattgattaataaggcagagaagaaatttttgttaattgcaattactattgctttccaaagagagcagactccaaggataatcccgggatagtacagcccacagatccgaaggtgccttattcccaggcgcagcttccccacagactccgggtgttcaggaataaaccccgagtcagggaatgaaatactcctccagcatcaacctgagggccaaggccctacctccacacccactccctggctcggtgcccatcagccaagtccaaacccacacaagttgttcccactctggaaatggtgggggaaaaaaaccaacatccagtgccagaaatcagtgtgatgtacacaaaagctgcttttttgccttttgcagatgcagagtggctgcccaggctcacgtagccagagagcaggccctggggagtggtccaggaaagcactggacagtagtcatctgtctggaaaatattagcccacagtctgatgtcttcaaaacctgagccaggtcaccctttctctttcactgacagaaataaacagttcttggtcttgaaatacacaggactttccccagttactgctgctgctagcactggcttacttagtctttcatggtttagagcctgttttcctccggaagcaaaatttggcttgagcgatatgaagagtaatgaaaacattttaaaatgcaaagctgggttctttttcccaagggtagcttcttggaaatctctggtttttccagcaacagctgaaaaattgaggcctaatggatactctgtgttacaaagtcagagaactccacagatggaaagaatcaacctcctttccctggatgcccttcattttagagctgatgacgccagatgacgctagatgcctagagcatctagacccactccccttcatttcttcctcctgacccatagtctagagactttgaaaacactacaggtgttcagattccatttaaagactagagctcgacattaaatgcagccttcctatcactatgtatgggggagagatagtcttttcactaaatggtactggaacaattggatttttatatgaactataatgaacattgactcctacctcaaatcaaacagaaaaaggaattaagacagatcataaacctagatgtgaaaagtaaagcaataaagcttctagaggaaaacaggagaatatcttcatgacctttgggcaggcaaagatttcttaaacagaacacaaaaagcactaaccataaagaagagattgttaaattggacctcattaaaattaaaaaccatccttcattaaaagatattattaagaaagtggccaggtgtggggctcacacctataatcctagcactttgggaggctgaggcaagagggttgcttgagcccgggaggtcgagaccagcctgggcaacatggtgagacaacatctctacaaaaaaaaaaagtaataataaccaagcatggtggtggatgcctgtagtcccagctacttgggaggcttagatgggaggatcacttgagcccaggaggctgaggttgcagtgagctgagccaagatcatgccactgtattgcagccttggcaacagagcaagactctgtctcctgtcttaaaaaaaaaaaaaaaaaaaagtgaaaaggcaaactgtggactgatatctaacaaagcatttgtgtccaaaatatatatcaaacttctataaatcaacaagaaaaagacaagaaaacgacaaataattcaatgtttagaaatactagaaaattcttacttcacaaaggagaatatccaagtgaccaacaagcatttgaaaaggtgctcaacatcattcatcttgaggaaaatgcagattaaaatctgcaatgagataccacgatgcacctacctaaatggctatagctaaagaatttgactgtatcaagtgttcttgagaatgtgaagcaactttactagtggagtgtgctttggtaccactgctttgagaaactgcttggcagtaactactaagacaatagcacaaaatgtgtgcataatatgttcaccaaacacagtgcagcactaatcattgcatcccagactggaaataacccagatacccataacaggagaatgggtaactgaatacatagtgctttattcataattatttctaattctcatcataactcattcttccatgtcgtatattcatgattatttataacaaaatattgtacagcagagattggtgaactgtgacccacaggccaaactgtagcctactttttgtttgtgtgttgcttattactgcatttgagctatgatggcagggttgaatagttgtgacagagactatagctttcataaccaaaggtatttattatctggttctttgcagaaatagtttgctgaccgctggtaaacagcaatgaaaaagacagactctctattatgtgcaaaaacatggatggatctcacagacataatgtggggtaaaagaagccaaatatggttgggcatggtggctcgtgcctgtcatcccagcattttggggggccaaggcaggagaatcacttgaacacaggaggtgtaggttgcagtgagctgagattgtgccactgcactccagcctgggagacagagtgagactctgcctcaaaaaataagaagccaaatacaaaagagtacagcccgtgtgattccatttacacaaagtccaaaaacaaacaaaattaatccatggtgatagaggtcagaatagtagttacagtagtggcagggaggtatgaggaaggggctcaaggaagcctatgctgtagatcttgatgggggcagtggttcaacaggagtataattatgtcaaaatcaatcaagctgcacaattagattttgtgcactgcatatcacttctaccctaatttttgaaaaggggatcaattgctggcctccaagcagggcctgccacatgcaaagcagagtgatgcagtttcctccttcaccccagagattggacgcagtaatagtagggccatgtcacagtccctttctgaatagagcacccccagatggtggctttcctacccacaggccaccacgtataataccacttgaccccatcaacctggctccaaattcctggatccagctgatggtgccaaagccaaccttccatgtctggtcctcaaactttagtccacttacaaaccacctgggggcttgctagcaacaccgattccaggtccctcttctcccaattcctagtttttttgagtcctgggtgggatccaggaaactgcattgtcagcaagtcctgcaggtgattttgatgcagatggaccagagactacactggagaaacgctctgtgcaggctgagggagtgggcatgaggaggcaaaggggagaaggcaagagggtgaaattgctcttgaccagaaagggaagcaggagagagctaagaggaagatgtagcggaagaggaggtgagtgaggctgagggagagcaggcgatgaggtggccttgatgaagtctggccatgtggtgtgtcatggaaaacctgtttcctacctctccaccaaagagttggtaaaatatctgtttatcaagggaaccacccccaatatttcaacgtaagttctcttctatttccctaagtgtcagccagtctgagaaataaagggaaagagtacaaaagagagaaattttaaagctgggtgtccaggggagacatcacatgtcggcaggttctgtgatggcccccaggccgcaaaaccattaagtttttattagtgattttcaaaggggagggagtgtatgaatagggtgtgggtcacagagatcacatgcttcacaaggcaataaaatatcataaggcaaatgggggcagagcgagatcacaggaccagggcaaaatttaaattgctaatgaagttttgggcacgcattgtcattgataacaccttatcaggagacagcgcttgagagcagacaaccagtctgactaaaatttactaggcaggaatttcctcgtcctaataggcctgggagcgctacgggagaccggggcttatttcatcgcttatccacaaccgtataagacagacattcctagagcagccattttagagacctccccctaggaatgtgtctctttctcagggctgttccttgctgagaaaaagaattcagcgatatttctcctattcgcttttgtaagaagagaaatatggctctgttctgcctggctctcaggcagtcagacctaatggttatctcccttgttccctgagcatcgctgttatcctgttcttttttcaaggtgcccagatttcatattgtttaaacacacatgctttatgaacaatttgtgaagttaacgcaatcatcacagggtcccgaggcaacatacatcctcagcttacgaagatgatgggattaaaagattaaagtaaagacaggcataggaaatcacaagagtattgattggggaagtgataaatgtccatgaaatctttgcaatttttgttcagagattgcagtaaagacaggtgtaaaaaattataaaagtattaattcgaggaactaataaatgtccatgaaatcttcacaatttatgttcttttgccatggcttcagccagtccctctgttcagggtccctgacttcccacaacatctgtttcttattaatatgctatgaggttggcatttgcagggtgtgttgaagggctgagtccacctttacagggtgaaaaaagaggctgtccagagaggttctgttctgtcacatgtggatttgattactgtagaggaaaatgagtatcaccctcctcccagcacttgggctcagtcattcccatcaaaccttctgttgttttttttgtttttgttttctggtttttggtttttttttagatggagtctcactttgttgcccaggcaggagtacgatgactcgatcttggctcactgcaacctccacctcctgggttcaagccattctcctgcctcagcctcccaagtagctgggattacaggcgtgccaccacgcctggctaatttttgtatttttagtagagacgggaattcaccatattggccaggcttgtcttgaactcctgacctcatgatccacccaccttggcctcccaaagtgctaggattacaggtgtgagccactgcacctggcaaccctctggttttcttcatttggttcaggttgaaagttgcctcagcccatattcagatacatatatgtgtaggtgtgtatgtagattcctgagcaggcccctatgggcaagaccaaccaagccagatgatgtgttgtgaatgaaagagtgggcatgagataaattacattaaagaaattgacgacgtcttttgtagcttacagacaaacagcaggtctttagggactgatacgctataacctccaaggtagatatttaagtttaaaaagcaaggtgttttagtataatatgatttctgaacaattagtgggggagggatgcatctgtaggtttttatacattaaacgtcttaggaagaaacattgaaaactagcagagtgatgggctccaagaagagaaattgggcatggaagataatcaagcttgggaataagacaattctttttaaggaacagggtctcattctagctcctggctgggggcctcctggctggagggcagcagtgccattatagttcactgcaacctcaagcttgcaggctcaactgatcctcccacctcagcctccctagtagctaggactacaagcatgcaccagcacacccagctaatttttaaaaagtttttgtagagatggggtctcaccacattgccctagttggtcttgaactcctgggctcaagcaatcctcccgcctcggccttccaaagcactgagatcacaggggcatgagccgctgtgcccggcccaacttttattctatattctttgatgctgtttttttccctacataaattttatctatttaaaacattttaaattgagggaggaaagaaggaaaaaaggacaagaggaagggaaggaaggaggaaacagtattcctagatgggtattggctcctgaagagccagtgcagagttgagagctgttccagcacatgaagcaagcacgggccctggaggatactcctctggccaccactgatgcccatgggcaggaagccaagttggttcaccaagccagggctccctcctccatcacttcatccatggactgtgggaagactgtgttgtcttaaagtcatgcccacaccctgggtggaaatgccgtggtgtactaatgtggaatttccagcagcttctctagcacagtacagttcagagattgttttctttgtgttaacatggccaaggttgaagcaagaaggctgaagtgtggaaagtgcagaaatttggaaaatacatgaagggctgtcaaacggcctcctcctggtcactgtcagagcaccaagacctgacaggcaggatggcagtggcaaaggctctaggcccagactatctggaatcaaatcctggcttttctacttgcagactgtaagaccctcccgagcatcatttcccctgagaacctaacagaatctaccacatagggctgtggtgagaatgaagtgcattaatgtatacaatgtttaagcagggcctggcagaacaaacacacagcagatgttagctgctcaggctattgcatcttatctgaattctaacagctccttgtaaagcaggttactttgatactccgaagcttaggtaaatagacttcataagggctacccaacggagtctctgctaccaagcccagggtttggcccacgacatcagcatttctcaaaccctatttgagaacccttagtatcccaccaaaattttaataaatactactccaaaacagggttttgtgaccaataagtttgggtaacactggtttaagtgaaattaagcaggttatttgctgcagaatttatcagaacctttacagtgccaatgaatgtgtggctcttagatggagtagcatttcccaagtcattcgaccattaaataattttctttttgtagtacactattaacacctagaggaacattagggttgtatatagaaaataactttggaggtgctatgttactgtataggacaggcatggttagaggaaatcatctgttttcaggccagtctccatcaaagagaacgtcttcaaagtagactgtagaacatacctgggcatggtggttaagagggagataatgagttgtggaaagaacatgagtactaacagcgggatctgaattcaaattctgactcctccaactaggggaaggcatttgctttcgcagaacctcaatttcctcatctattggttggcagactagcatctacttcattttgttgatttaagatttaaatgtcagtggccccaaagtgattgccacatgatagtttctcagggtcagtttcctttttgtatcagtcaggatgttttcagtggcaaatagcagattgccaaacttaaattggctcaaacaaaaaaaaaaagcggggggaatttatcaacgtaagcaaacgtccaggttggggtttatcagagtttgtggtgtccttagagcaggtgggaattctccccaccagagagcttaatgcccaggcctggaaaattctagactgacaattgtatatagatgatctgcaagtgccttgatccctaggggtcaacacatatccaccaaaaacaagtcatttgcacttttgcagatcatctttagtttcccctttttcaaaaggattattttttcagggggccgtgatgaagcaatcagtctcaagccacgtgggcaggcagccccaacagctatgagaattttcagaaggagtttacaaaagcatagcccaagagcctacagttttactaatagaaactgctgaaagtaaagcctacaaaatctctttggcttgtagaaatattttaaaaactgaaataattaattattgcatagggcatgggcaataagaaataaaaagtttaagaagcacaaccatggatgtactgtagcagaataatagcagctgccactggtggaaacactgctatagttccatgtaaaacacatagaattttgacttttctattaaagcacaattttaagtgtttttattaaggtgcattagttgtatgtatgctgctcagtggattttttttttttcagtttttatttctttttctcagtcagggtctcactctctcacccaggctggagtgccgtggcacaatctcggctcactacagccttgacctcccaagctcaggtgatcctcccacctcaggctcccgagcagctgggaatacaggcatgcaccatcacacctgactaatttttgtattttttgtagaggtgaggttttgctgtgttacccaggctggacttgaactcctggcctcagctcagtcttccaaagtgctgcgataacaggcgtaagcctccgcaccaggccagctcagtgaatttttatgcacatacacatacacatctatacctaccattcagatcaagatataacacatctccagctccccttcatgaatgcaccagttgatgaaagttcattcatgctccttccttgtccatcccctgcttcaggaaccagtattctgattcattttgcctgttcttgaacttcataaaagtggaatcacatagaatgtactcttgtgtctggctttcttagcttattgttacagccaagagaggtaccatactgctgaatgtattattaatttcttctttttcattgctatgtaaatttatttgatagacattagggttttgcagacattttgttgctaccttttatcataatgtctacaaaatacttatgctaacatccaatttagtggtaaactattgaataattttcttctgagtttgaaaacaagacaaaaatatctactactgtctgagtgcgggtgactcatgcctgtaatcccagcacttcaagaggctgagacaggtggattgcttgagcccaggagttcaagatcagcctagacaacatagcaaaaccctgtctctgcaaaataaataactaaataaaatagaaattagccaggtgtgttggagtgcacctgtagtgccagctactcaggaggctgaggtgggagaatcactcttgcctggaggtcaaggctgcagtgagacacagtggtggctctgcactccagcctgttgacagagcaagactctgtctcaaaaaaaaaaaaaaaaaaaaattctactatcagcagttctattcaatactttactaagagacctgagttctggcccttacccctgcctctaactacctagggaccttgaataagtcaattaaccactctgcttcttagtttcatcatctttgaaaataactagataatgcttaagatcccttctgcatatctaagttccaagtaaatgcacctttttttaaaaatgtgataccacaacatggatgaatcttgaaaacattattctaagtgaaataagccagacacaaaaggactttttccacttacataaggtatctagcatagtcaaatacacagagacaagctagatcaactaggaggaaaaaagagagaagactcaaataactaaaatgagaaatgaaacattagtactaattttacagaaatcaaaagattatgacagtgctttgaacaatcctaaaccaacaaactggataacctagatgaaacggacaaatcctagaaatacatgctaccaagattgaatcatgaagaaaccagaaaatctaaatagacctatagttttaataaggagatggaataagtaatcaaaataactcccaacaaataaaagctcagggccttatgtcttcattgatgaattctaccaaacaattgaaggagaattaacaccaatccttctccaactcttccaaaaaattgaagagaaggaaacacttcctaacttattgcatggagccctaataccacagacacaggcattacaagaaaactgcagaccaatacccctgatgaatattgacacaaaagttcttaacaaaatattagcaaactggatttaacatcatatttaaaagattatattgaaagaatgatccagagtaattaggcaagaaaaagaaattaaaggtatctaaattcggaagtaaaaagtaaaattgtctgttcacaaatggcataattgtatatgtagaaaaccccaaagagtccacaaaaaaaactgttagaattaacaaaggatttggcaaaattccaggacacaaaataacacagaaaaatcagctgcatttctatacactaaaaatgaacaattggaaaagaaaattaagaaaacaattccatttacaatagcaccaaaaagaataaaatacttaggaattaactgagggagtgaaagacttatacactgaaaactaccaaaatatagctaaaaaattaaagaagacacgattaaatgaaaagacattcagtgtcttttcactggattggaagactaatgcagttaagatcaatgcaattcctatcaaaatcccaaatgatattttttgtagaaatagaaaaatttattataaaattcatatgaaatcccaaaggaccctgaatagtcaaaacaactcgaaagagcagaacaaagttgcaggtttcacagacattttttccaccactgtgtgaccttgggcaagttactgaatctctctgttcccaagtttcctcatctgtagatgaggataacaacatcacccacatcatagggcttttatgaatatcaaatgaataaggatatggaaaatgcctacaacacagactgaaacatagtaagtactatatacagaatagcagctattattaattttttattaacattactccctggatggctgggggagcctagagaatttctgcagagatatctggctcccttgtaggtgatttagccaagtggtacccatttctgagaagcatattttttctagagtcaagtcaagacttagagccaagtgccaagaaagaaaattagccagagggctgaatcacttgactcacctggacagggaaagccccacatctgctatgctcagacctgcacagggtgccgcagaatgcaccagtttaataaactggggaaattggggagcttgtttcctgtcctttgacttgactttggcagcaaggggacagccccagcccttgcttcccaaagccaactcatgcccttccattgaccacaaaagagcacactgtcctagagacaagactgttactcaccaccgctgccattcacctggcaccctaagaaggaatctgtgccttccaaaagacacacagctaaaaagaatcaatcactgagctgtccccacaagtggaaactttctcattaagaaggctggaagcaggagatggagtgtctgcatttaattctgtaccctttccttgtgggaagcattggttgtcaatccagagaagctggaatcttccctgggaatcgccgacaagagtgataggggtcactgtgattctaaacactccctacaggcatttgcaatgcagttctcacagctg

SEQ ID NO: 5 - DNA, 43763 bp, Homo sapiens
ataactttgtagcgagtcgaaaactgaggctccggccgcagagaactcagcctcattcctgctttaaaatctctcggccacctttgatgaggggactgggcagttctagacagtcccgaagttctcaaggcacaggtctcttcctggtttgactgtccttaccccggggaggcagtgcagccagctgcaaggtgagttgccttcatttctggggaagcggctgttttgagagggttttgtttcttcctctttgagaaggctcagaaaattgtgggaattttctgcctacagagagaaggtgttggaaagtctaggtaaaaaaatgccgacatgaggtttggtcatttgaacatggcatcttggtcagatttcttttctgcaaatattagctgtggttgttacatgacaaggaaaaactttctgaaaagttcaaaatgggagtttgtatctgctaagaattttttttaacagagattatcttatcagtccttataacatgtaacctccccacaaatcctggtaagatggatatattaaacccattttatggatgaataaatgagtttcagttaaataacttgatccaggacacataactggtagagctaggattccaggatttgaacccaggtctatggggctataagttcttttctttctttccctctttccttcctttccttcctgtcctttcctcccttcccctcccttcccttccctttctttcttttttttttttggaaacagccttgactgcccagcctcaagcaatccttctgcctcagcctcccgagtagctgggactacaggcacaagacaccatgcctgcctggctaatttttcaattttttgtagtcaaggtctccctatgttgcccaggctgaactcgaactcttggactcaagcgatcctcctgcctcggtctcccgaaatgctgggattacaggcgtgagccactgcacttggcctaaaattcttacatggactatattcactacactgtcttctaaagacagagtttttgcctctttgatcttcctcttgtctgtttcaaaggagatgatctcagatttcgagggatgagtgcatttttcacaaatatttcctccccacataataaaagaggagcaagtaataatgctgagttagaacaacaactgcaaaatctttaaaagaaaggtagctacagcttgagtgctttccccagtaatagacctaaaactacagttgataatgagagctgctgggaagtgtctttcagggtcaggcaaaggcacccctgtttcaacgacctccgggctcttcctcaccctgcctgtggccagtgatgaagcctggctgggtctggtgggccagcaggtcccgctaaggaggctcatggtgcatacagtttgtccttgttaagtggcgatggggatgggggggttcattgtttttccttaagattattaggtggctttacagaattgaagtctgtgtccgaaggctccccattccaaccccgaaatagagacagaggttgcaaagcattgatgaaggggctcatgctggcgttcatatcgattccccagtgagccaaccccactgagaagccctgttctgtaaactcttcttcaaccttccctcacctctgcttcccttcttcagccccacagtgaagaacatctgagctcaaatccagataagtgacataagtgacctgctttgtaaagccatagagatggcctgtccttggaaatttctgttcaagaccaaattccaccagtatgcaatgaatggggaaaaagacatcaacaacaatgtggagaaagccccctgtgccacctccaggtgagttttccctgggggaacctcctgcttgtctccttgggcacccatgccctgtcggcaccgatgactcagaattcctctctctgctcctcctctctcacctgaggtctgatctcctcattgcttcttgaaagtccctgttggaattcaatgcagccgctgattattgggcacctcgctgtgcctgacggtatgtgaagaaggaagggacatggtgcggggctggccctgaagaagcttggaatctttggggaagaatatgggaaggagcttggcctgagaatgggaggcagtagagcctcgtggtgggcaccatggcggaggggtgggggtggagggtcagcatcctggcctgcgtccattccaattcaccgcttgctagatgctttgaatatcatcctggtgaagggcatcaggatgttagacaaactagttccaagccctgtgctggccgcactagttgccgggtgctgcctctagccagctgtgtgacgcagggtaagttaacctctctgaaacttagtgctcacctgtggtatgaggaacccaagggggccctcaccatagtgtgctgaggatgaaatgagatctacagcactcagcactgtgcccacacagaacagacacttagtccacatcagggttcatcaccatctactgagtacctgcccttgccagagctacacaggccacttgacagactttgcatctcaactgatctctgtggcggctctttgaagttcatatgattgactcatttcacagataaggaaatagaagctctaataggattatgacttcccccagcctgaatgaccggtaattacagagtttacagagttggaacttgagcccagatttggattcttcatgtccctcctccccgggcccatccttcctcccagggcagaaccaagagaatgagtcagttaatgaacaagtcaagggacaggcttgaggctcgctcaggtggaaagacaaagcaatctgcaataacgcaatagtgcaactgaaaccaaagcccccatctacacaagaccaccacatagccacacaaccccaccgccccagaggagcctttcccatggactacaaagggtagggcgccccctggatgtgtacagcatgacggccctgagtgtatctgtcagaggcggcaaaaaatgccagtggctaagaggatgggcttggggatgagcaagtggctttgccactcagtagccctgtaacctttggcaagtcccctagcctctgagcctcaatttcctcctctataagaagagggtaacaatggtgatttcctggagttggaggaaggagtaaatgagctttcccatgtgagcaacttagcacagggcctggcctggagtaagtgctcagcaaatggcactgttggcattgctatcattagcggtggtgttggcagttaggaggtgatggagcaaaattgaacagagttccctctaggggcttcacagaagtcaggtggactctgtggcctttgaagtatgggtagaatgtagacaggtgcagaggagtagcaaggacattcaggaagaggggagaggtgagcagaggctcagaggcagaaatgagcctgactgtgcttggccaggagtgggctgggggctgggataggcaaagccaggccagagcacaagggcttgaatgcagggtaggagatttggactcgctctccctggcagtagggagccattgtaggttcttgagcaggacagtctagctgtggtgtggtaggtgaattggaggagaggactttgaaaccaggcagtctgggctatggcaactgaggtggctaagagaccaagaggtgagaacacagagttgatggcctctagatgtccagattctaagggctgtagaggggcagtggcttcgccccttccttctcttcatctcatcattgcagccgctgccagctgacctccctgactccagctccccacactggctctgagctctctcttgccaactggacttctagcaagtcacgcccctattctagagcctgcagtggctccccattgcctgtcaccacattaaagcttccacagagttctgagcgagcaccctacccaaatggagagcccatcccacctgcccatcccagccttcctcagctccccagtgcctgtgcatagcacattcctccctcagctcatccagacccaactcatcccctatttttttcttttctatttttattttttattattattatttttagacagggtctcactctgttgcccagacttcagtgtagtggctccatttcagctcactgcactgcaacctccacctgccaggttcaagcgattctcccacctcagcctcctgagtagctgggactacaggcatgtgccaccacgcctggttaatttttgtatttttagtagagacggtgtttcaccatattggccaggctggtctcgaacccctgacctcaagcaattctcctgccttggcctcccaaagtgctattacaggtgtgagccaccgcacctaggcttcttttttctttccttttcttttctctctttctctctctcttttaaagctgagaatcttgctctgttgtccaggctgccctcgaactctgggccctagtgatctatctgctgtctttccggatcaccctgaatctcccaaaacccttacggtgttggctgcagcctttaaccctcatccccacaccctctaatagcattgccagttatccctatgtctttgggggctgacttgtgtctttggaccattcaggaaatatgagttcagactccagggagggccctgagctgcagtggacatgtgacaaaccagctaacttgaccatgtttttccccctcctcttcatgctggggtgggctggcggaaaggatccagtcagatagaagaggggatgaagaaaaaaatatcacacatcaattcattcaactctcccaacgatccacaaggaaggaattttagttatcccattttaaagatgaggaccctgaagcccagaaagttaaagtaattttcaagatctcataattagcaaatgacagagctgggattcagatccaggcagtccagagggacccctgcatggggctgccacctttgtgtcctgggcatgtcacattccattgacgagtacatgcagatgcccactcccctggctctgtgctgggagttaagatatcaatgaaacccttctgctcatcagtgaattgtttgagccacttattacttacatacaccactgctaaaaacaatagataacatttataaggtccttagtactgtgtcaataatgagacaggaactattatccttgttttacaagattgagtgacttactggaggtcaattcctggaaaatagtggagctaaggtttgagtcaagtccaaactcataacatctacactatctgtgctctcccagagggcctactgtgtgctgcactgttcaagagacagagaaaaatgacccagttcctgctgtcaggaaggaaacggggaaacggatgtcttcctcgctggtttgggagacgtacatgaagaatgcgagttggagaaattgtgtcaggagtaggtgggatggagtgtagacaggaggtcgtagttgtgggtgtactgggaccgtgctagaggcctctgctggggagactgcagaggtgcctgggcaggctgtagtgtggggaggagttggggggaggaggtgggtccacccagagtgcaacatccactcaggtgagggaggggtgatgttactggaagaggaagcagccgtgcaaaggggatttcatcctgaaagcaatgagagccaatgaatatattttgattttaaaaagtaattttctaatctgttcaggtgtcatgaaaccaagagcctattctaagtgactggtgattctataccttcgtaaacaattaggaaatgagcatggtgtgtgtgtgtgtgtgtgtgtgtgtgtgtgtgcgcgtgtgtgtgtacgcatgtgtgtattttagggacaaaggcaaatgtgggagcatctaggtgaagacacatggaattcagaaccaagtcgagctatgtgttgcttgctgggctgcagttggctagaaataaatatgtgccctctttcattctgctttaactggggttcgggatttaatgtgcttgcatactgccagaagggccccctgcatgttgaggtccagggctgctgccccatctgggtctggaccctacctgttatcttgcttgagataaggagcaccttacaggcggagcaaagtcaggaggctctgcgcagtccaggtagcaaggacaaggggcctgcaaggcaccggaaggggcttacgatccctcctgcaatattcctactagaagtcacccctaacccagtgctttctttggtttcttttctggaatcgcacagaacatgctgtatgtaacaagtaatataacacttcccgggacagccttgtgaggtggtgtgagcttaggctgggctgtggtcctggggctgctgtgtggccctggacaagatccttaatctctctggccccagctaatgcctcaataaagtggagatgacaacgtagagctgtctgcttaacgagactggacgaaatgacagaaaggaaatttgctctgtaaactagaaagcaccgcgcaatggtggtaattatcataagctttattgcgattttatggggaataaacaaacacgaggcccaacacgacccgggcatcttagcccagacacagtccagtgccatccaaactccgcggcagacgccggctgcgggcccctgggtgcttgggtcccgggatcgccggggaaggcagggaaggaggggcagctcggcccgggacatcgcgtgggtgaagagcccccgccgctgctcggctcgccccgcccccggggcgggggctgcaggctccgagcgataaagcgcgggcgctgggaggcgcgtggagccagcggagctgcgcctaaggagccgggccatgcgcgaggggctgggcgcggcgtgcgaggccgccgggccccggctgtgcaggctgctctgcaggatcctccggccgaagcctgactgctgcccgcgcagagccaggtaccccgcgggccgaagtccgccctgggagggaagcggtgggctccgagcccgggcggggccccatctctctctgggagaaatcggagggcggggatgtgcgctctgtgggattcctgagggtttactaaaagcgttttcctcttttagaacgcttgagaggtcgcaattcaggagcccaggcggcccagagaaaccccccatgcactatgagaggcgtctatgcgggttcagttgcgctgcgaatgtttcagcacttgggagcttttgaaatttttcagcactggggttgaaagttgctacactctttggatcaaatatggttaagagagcaaggaggttcttgtttcacaaatgagatccatttcccagggcgccatcccccccaccctccccatctgtccccaccaccactcttggtgtaaggcgaggtaaacaggcagggaggcgtcggaaccgcaggggaatctggcccccaggtcagcccaggcaagccacagtctgcagctgtgaccggctcggctggtcaagggcttgtagaaagttctgggtctaagcaagccaccacactcacagcactccccacgcccccgacccctttttttgatagggtctcactctgtcacccaggcgacagtggcgccgtctcggttcaccgtagcctccacctccagggctcaggtgatcctcccaccttagcctcctgagtagctgggactacaggcgcgcatcaccacacacttggcacctttttttttttttttttttttttgtagagatagggtttcaccatgttgcccaggctggtcttgaactcctgggctcgagtgatccgcccacctcggccttccaaagtgctgagatgacaggtgtcagccaccgcgcccagctgccagcactctctttgaatgcaggtccttgagggtcctcaggaccccgcacagctgtgggtttagtcaaattgcacgagcgtctgcggggtcagaatcaacaatgtgataatttggaaagaagtgtccagattctgaaggtgctcacctggagtctggtcaccctgtcaccctgtctcccagcagccagccagaagaaatggtgtgggaggggacttgggcaaaactttgtttattcatttttcaaatttaaagaattgtccaattatgaaaggaattcattgtcactgtaaaaagttggaagatgtggaaaagtataagaaaagttaaattgcctatgatcttatcacttaatgctagtcactgttaacatttactaacattccctcgaggtgtttttctgtgcttgtacgcacacacacacacacagccagcatcagactgtgtatacagtttaaggcattatttatatgagggcaaaactagacacagtctaacatccagccatggacaatgggaccaggctaatcgtggcccatctgtagagtggatttttagccaacttttaaaagtgatactttttaatatatggggtaaaatgattatgttaaatgtacaaagacattgcttctcaatctaaacagtactgtagatgtttctggtctgaagcttctgaaattgtccaactctacttccagaagtaagtgagaggaagcaagaatgggtgcagggtgggtgctggggtgagggggaatggcaaaagagcaagaggggcagttgtcccctggggaccccatcccccatacatcttcctattgaagcagccccatggtgactgctcagccatggcttatctgttctgaatatccagattgccatatatatatatatttttgttttgttttgttttgttttgttttgtttttttctggcaggccacaccagtctagaccaggtgtgttaaagaaaaaagtattcaatgatacttgttaaagaacagtatggaagtctttatttaggaccatctcaataggtacagggacctactgcagtggggtctggcagtggaggagagagattgggcccaaatccaaatacagcaggagcaagtgggaatttatagccaaggatcagggtggggtctgtggatggaaaattaccaagaaaaacttcaggcatagggggaattctgcctcacccaacctaacaggattcttgcttaggacaggctaaggtgatcagacatcgcctgggggaggatggaggatgaggaccctgatcagatggggaggacgacaagctatcaaggatggagggttcttgttaaactgactaagtagggattcttttgctaaaactgcatttttacaaggaaatgcttggatgggccccgcagaagattcagaagtctgagtaaagtttggctaagcaaagcatctttgccagttggcaatggttggtcacatggaaggccctttacagggttgtggaatcccttgattcagatataagttgctttctgatacatacttacatcctcttttaagacagtatctctttatagtaaaaaatcacttactgctactatgtaatgtatataattaatgatataatggtaacatatagtcatttttttctataataaaagcaatacatggtcatttatcgcatttcaacatcatagatacatatcaagtagaaaatgtgagaccctcataatagtctctaaccacctctccctcagatgcacaagcatgcataccctggaaaatactgccaagaacatggaacacaatatgctttcaaaccaaggtgccctggtgggatgattcttcctaggggaggcatgtgtgagtggtgactattcagttcgccggaatgaacctgaaatcccaaaccaggacaaacggtttctgaccggtttcagaatactgaatttttaagtaatctttaatttaaaaaattactttaagtaatgtttaatttaatttgtaagtaatctgtaatttaatgctaatttttacaatacttgtttttaccttggtgaaggtaagttgacatcttagatttgttttaaaataatgatgggggcagggaaggtataggtgaaacaagatcggccgtggattgatcactgttgaattgggttgaatctgggtgaagggcccatggaaggttattagactattctctctacttttgtacacttaacattttctgtaatagaaatgtagaataagtaaataaaccaagataaatcacgtgttttaaataaagcttaagtcatgaaaaacaagtggctggggctgggcgcggtggctcctgcctgtaatcccagcactttgggaggctaaggtgggaggatcacttgaggccaggagtttgaggccagcctgggcaacatggtgaaaccccatctctaccaaaaatacagaaattagccaggtgtggtggtgtgcgcctgtagtcccagctacccaagaggctgaggtgggaggttcacttgaacctgagaagtggaggttgcagagagctgagatcatgccactgcactccagcctggatgacagtgcaaggctctgtttcaaaaaatataaatcaatcagtcaatcagtcaaccaatccaagtggctgctgggcagtgctgctagtctgggaggcagagttgttggagccattctctgcccacccagcccctgtgccacgtttgtacttggcttttccagagggtgaaggagcattttccagatcctctatgggctgctgtttgggccagtagctttggctcagctctgctcatgcccttttggctctgcgggaggaagaagataagaggaagagggaagatggggatggggaggcagttggtcagaccgaccatgaacaattcactcccaccctctggactcagtttccctatttgtcaaatgaggttactggcagagggaactccaagatgcccctccacccttggataccgcatctcatgctagctgtttcctttcacagtccagtgacacaggatgaccttcagtatcacaacctcagcaagcagcagaatgagtccccgcagcccctcgtggagacgggaaaggtcagtgagtcacatgggtgtgggaagccccttcctccctggtccaggcagatgcagacatagcagcctgttagagctgggctggagagggcaggaagccagggtttggatggagagtggagagtcccagacccggggtgttggggggaacaggtgggaatgaggaagggctttgggagagtgagacaacccccagtggtcgggagtggtggcccccaaggtgcactgacagctagcttctatctcctgggttccataccattctagagaggtgggcagagaaatgggtaggagtgggagagagggtggcttatatgtccttacatgctcccagggaaggaatgcagtggtgcctttctgttctcagtagtattggctctgacagtcatccttctcacctccaacttagtctttagtccctcctacagcagttcctctgaggaagcatttggcaaggccctgggtgaggagaggaccctggccagtggcccaggcacctggagctcttagcaggcactggcccaggtcgcccgggggctggaaacagaggagggacgtggagggacaccctgccctgtgtggccacccatagatccttccccactctaagacaaggcagggcctggggtctaggagagacctgagagaaaaccctgttttttccccttgcagaagtctccagaatctctggtcaagctggatgcaaccccattgtcctccccacggcatgtgaggatcaaaaactggggcagcgggatgactttccaagacacacttcaccataaggccaaaggggtaagtctggggcttcagggagaaccacctttgtcccaagctggtgaagctgccagggctttgtggatcaaacaggtgtccccctgggcaaaccaaacaatcatctgcttgggcaaaggggtctaggaacagagacattcctcacagattacagtaggaagtaaatgatgccaactccctgttaaccaccccagtaaataatggataatccagaaggtcaaataaatgattgcagaaacaaattcatatctttcttagccggatttcaggataatgtcagggagaattttctaacagaactgctcagagctggactgtattacatcaggaggtagtgagctccctgtcactggaaggatttaaatgcaatttgatagattgcagaggggttcaagtatcctgtgagtggttggactaaatgtcttttcaggcttctttctgtcatgtaagtttttaattctaacttcaggatacattctatggttgtaaagtgggaaagaatggacaatttcaggaccatgaaagcaaaattatttcccacatgcccccaaaccaccacagttaacattcgggtgtacttcattcgtgtgtgtgtgtgtgtgtgtgtgtgtgcacgcgtgtgccttcctatgtctcatttcagtttgttttggccaaataccaatatcatgttcccataggagccatattggattgactttgctctggatctggagctggctggggagcagggccagagactgagtccggagtagggagaaaaacctgactcacacccactggggttcagccagtggaagagaggagatctaggtgggagaaagccaggtggaccccaaggaggaggtgccacaggggtggagaagaggtgcagggagccggagcagccagagaggagccaggtggccaggagagctggccccagaggagggtggcaagaggccgaatggcccaccctcaccacctgcctctctctttccagattttaacttgcaggtccaaatcttgcctggggtccattatgactcccaaaagtttgaccagaggacccagggacaagcctacccctccagatgagcttctacctcaagctatcgaatttgtcaaccaatattacggctccttcaaagagtaaggcttgcctcctgcagggtcgctgcctcctgcccttcctcctgctctcctgtctctacccatcaccctctgatcctagctagatcccttttctttccagatttgtggtgttaaatcactatgattttgcagagcgaaaatcacggttttctaggggagaagaccctggagcaggttctcataattgtgggataacgttgtacatagcaataccagttaaaagtgactgaattcgaaccctggctcccgcattgtgtctgaacgtcctcatcatattaatggcaaaaactgcaactacttttgcaccaacctaatataaaatggggataataacagaaggtggctcatgagatttttgtgaggattcagtaagttcagaacagtgtcctggcatccagagaccttgattgtgaattaatatggaccctttaattatgaaatgtttgagacagaaaggatctctgagaccacctagtcaggcccctactcctacaggtgtgaacatggaagctggtgacatacctgaagtcacctcattccagctagaagccaggccacctgactaggagagggcaggccattgctctttgccctgaccctggtggggttctacatttctggagttctcttcttttttctacccagtgaggcttccctgaggaccatgctagacatccttcatttctatggccaattggtcatttggttgacagacacacatcaggcactgagtatgcctcagccttgtgtcagacactggaatacaaagatttttaattgtcatttgccaattttattgccaacgtatacaattttaatcatccaaagagaggttgtatagcattgtggccttcccaggttcaaatcctagctgtgccccttacgagctgagtaaccctgggcaagttactttgtctctctatgccttgattttcttaccgaaaatggtgataataatagtacccaccttgcaggaggagggtagttggcgggagtttataaagatgaaaggagttaaagaatatagagctaggaacaatgcctgggccacagtaagcacgacataagtattagccattttcatgtttttagaacactgcgttgtccaaataaaatatgtggctatgtaatttaggcccctttaaaacttttaaaacaaaaatgcatgaagagattgtaagaaagaatgtctgaaagaaatggattgttttctttttgtttgtttgtttgtttttatttttgttttttgagatggagttttgctcttgttgcccaggctggagtgcagtggcacgatcttggctcacttcaacctctgcctcccgggtttaagcgattctcctgcctcggcctcctgagtagctgggactataggtgtgtgccactacacctggctaattttttttttttatttttagttgagatggggtttcatcatgttggccaggctgatctcaatcccctgaccttgtgatccacccacctcagcctcccaaagtgctgggattacaggcgtgagccaccgagcccggcccagaaatggattgttttctaggctggaaggggagtgttgctattaggggagagcctcacacagcactgcaccacacaaagtgagccacagacatgtacctgggactccctggcggaaagagtggcttctggtggtaccacctccacttttctttgtcttttttttttttttttttttttttttgagactcggtcttgctctgtctcccaggctggagtgcagtggcacaatctctgctcactgcagcctcgatctcctggatcaagtaatcctcccaccacagcttcctgaggagctgggaccacaggtgtgcaccactatgcttagctaattttttaaatttttagtagagacagggtctcactatgttgcccaggctggtcttgaactcctgagctcaagcgatcctcctgccttggcctcccaaagtgctgggattacaggtgtgtgccgctgcacctggcccacctccagttttctacaaccctagaatgtctgagctgggagggaccgtgagtcttcagaagaggccctcatcttccagggtcggtggtcctcaaccctggctgcacagtagacttacctggaaagcctgcaaaattctgatggcagggtcccttgagccaagggcagcagaatctggggagatggggatgaggcagacatgtgtgtgcttcaaaagctccccaggtgattctaatgcacagcctggattgagcagcctgttaggggctgcctgcttcctgactgcaaacttcacaagatccttggcctttcagctcggttgttaattaatgagcctttcggggtccagggattttcctggatgggtttcattgggccacagtacttatgttatttatttatttatttagcataaagcacgtgtgcaccatgacactatgagattggtattattgttatcctcatcttacagacaaggcaactgaaatccacagaagagatgtaccttgctcaaagtcacacccttcgaagccccagagtggggattcgaacctaggcagctggtgcctgagcccttgcttttcccatcaatgctctagtacctagctgtgtggccttgcaggttcccaactctattctggacgtgggaggccttgctgttgtgtggaaggcaattgggcgggtgttcagaagcccagcagggctacagggccatgcttggtggtgttggaacccatttgcttctcccagaggaaaattgtatttttccaagtcacaagtacataccttgaacccctgcttccctgagcaagagaacctgggggtgtgaacggggaatggcagcctctcctcacagggaccctggcaatctaagacatgaccagctgagcaggttcagtggccttttgtatctgaagaatcatagcatatttagagattgtgacttgctgcatgttacatttaaatctgttcactgatgatgaaagccactcctttttttcagagctcatagacatcatagcatttctaatgccttagaaaatgactatatgaaaatggctatcacatctaaaactctgaaggaattggatgtgaatgtaaaaaataatcctttcttgcatatgcacgtgctgaccaccatgtgtgttggtggtgtgggtgttatggtgatcattactgttgtcatgctcaggtgcagaggggggaatattgctggaagctttgtttggggagggaggtctcccagcagtggggtggttcccacctcacccagcagctccttatggcaggggtgtccttggggataaatatcatcagtgttttgtctttgtgctttttttctctaatcaaggaaaagttgtcgagacactcttcatccacagaaaggccattttgttcagagttacccactctccttaatcaggagttcattctttaaatagttttgatgctgtttcaaaaggctttggtgctgactggaaatctcaaggttgttgttgttggtggtggtggtagtgtgtgtgtgtgtgtgtgtgtgtgtgtttcaaagcctttctgactgagtccctccctccctttgggatatttttgaagtctgggaggggaggagagaagcaagtttagaattgcatatggagtgactgaatgagcaattctgaggctgaggaacagcacggtttagagagactatgacgtgggcagctcttttctggccaggccctgggaccctgcagactgagctccgatagccaggtcccagtcctggcttgaccaggctcctgagaagggtgggcaggccctcaacatctttgggcttactgtccccgtagggtggaagatggaaggtcacgttgctgacagttaagaacctgggcttggcatcagggcgacctgggtctgagtcctgactttgctctttcctgtacttatgttcctgggcctggtgcttaacctcctagagccttgagatccttggctataaaatagatgcaaaaatacctccctcccagactcagtgagaccctgcctgtgggttgcttcacccagtgcctggctcatgtgggtacccagtacaccggagctgccacggagggactggctcatctctcagtcatggcaaggaaggagggcactctactaactaatggagaggcaggacattgctatggctggatgggacactcctcccaggcttgcaagtgtctgagaaggattgcaccccttactgagatgaatatgattaaagaaaaagcacctgaaaataagcagtgttcccatgggctcaggactggctaggtagtatttataggactcaatgtaaaaatgaaaatgtgggctctttattttaaaatgattaagaatttcaagatggggacagcagagcattgagcgtggggtgcttctaggctaagtgtgaggtccccaaagccagccctggagggagctgtctctggtgagaggcatcttgcccttctgatgtcctatttcagttcctgcttctgtggattgaattaatccccatgtaaggccatctcattgttgatcttcctgctgtcttcctgtcccccagggcaaaaatagaggaacatctggccagggtggaagcggtaacaaaggagatagaaacaacaggaacctaccaactgacgggagatgagctcatcttcgccaccaagcaggcctggcgcaatgccccacgctgcattgggaggatccagtggtccaacctgcaggtgaacttccttagacttgagcgggagagagctggaggcggccaggccctgagcagatggcttggaacagccacacagagctgtgcccaggcctcctgggactgaagccatggccctggatcctctcttacattcccaaatgttgagatggggcacctagggtccctctctagaagccttgaatcctgtttcagcaccagggtttctagctgcccattctctattgtgttaaattcaggagactaaatgccatcattagagtaaaccacgaccttgtttttattacacttgaggccgtggtgctgcaagatgtgttgttttcctccaatgcttgaattcttggctccaagagaacattttatttagcctagcctcctaggccatctttttgttggggagtcatttgttgcaggataatatcaaaagcacttggacagagaaatcgattagaactgccagtgaaaccaactctggctgtgtagtatgtgctggtgtgggaaaggacctagcctggggctgtggcaatcccgattcatggtgctttcaagtcaggttttcttggaattctgggtttgctggggtcttctgagtggggttgcctgtgctcccctagagggcagccatgaaatgggagcggtggtgcagatgggaggctgaattcttctgcatggtgttgagtggcaggtgcagattggaggctgcactcttcggcatggtgttgagtgtttgtgtgttgagtgtttcggctgcactattgattcccagtgacttctgatcagcctcaaagcctctgtctacccagtctttgatgggcatggtctggatgggctgttgcaggtcttcgatgcccgcagctgttccactgcccgggaaatgtttgaacacatctgcagacacgtgcgttactccaccaacaatggcaacatcaggtcggtgcacatttcccagggctgcagaggggcttgccttgcctgagaatcttagccaaaagatgcagcacttgggtctaggggaggcatctccagctccaggagaaaccagggagaaagaaaaaagagaaagactcaggcttcttcggggacttccagtagtgagcccagcccctgtgagctcagctcccatcggcaacccatgcccttggtttccttctgatgtggagccacttggaggggatggaggatctttaagaagtaactgcttcactattcagggttggggctcccattttttagaaggcatgggacattaagaggttaccttcctctcccaaccaacgaggtcaaagctaataggactgaacagctcatttggtcattcattcatctttttgttcagcaaacactcagtgcctacctaatgcaactcaacacaggacaaggctgggagagcagggggaagatggggtatgggggggtaggaggaagagtgactaggaccctaagatgctgctccagctggtgtgagcctatgacctgcactagacaggcaaccagctaactcaactgcaaggacggagacataggcagtgagggaaagactggcagatcagctaaccctgcctgatggatgggaaggcccccttgagggggtggaccctgggtcttgagggagggcagcaggagaaggcatgtcaggaggctggctcagtcagcttgggatcaggggctgcagagcaggtagggtggagagggaggtagggccagtgcaggatggctgggcccccaccagggctaggtggtaaacagtactgttcctgtcctgggaaggtcggccatcaccgtgttcccccagcggagtgatggcaagcacgacttccgggtgtggaatgctcagctcatccgctatgctggctaccagatgccagatggcagcatcagaggggaccctgccaacgtggaattcactcaggtacccggcccagcctcagccaccggccattggggcggggagccccgtggtgagcgagtgacagagtggagcccagaggagacacgcagcccgggcttacagactcacagggcccgtcttgttccccagctgtgcatcgacctgggctggaagcccaagtacggccgcttcgatgtggtccccctggtcctgcaggccaatggccgtgaccctgagctcttcgaaatcccacctgaccttgtgcttgaggtggccatggaacatcccaagtaagtaggctgagggtgctgggggcaagggcgagtcaacacacagcccttctcctttcctaagtgcggctccagccttccccataccttccctaaagcccagccagcagcaggtgacactcagggggctggccttcaaggactcttttgtcactgttgcattctgcagccattctgcactggcacctcgggcctcagacaggtggatgtgttgatgctgaggggaagcagaggcttaggcggatggggccccaggagcttgagaagcagtgactgcaggagaggaatgcattgagattctagaaaggagcagcccaggcctcttctgccctcctgccaatgctcttagctgtccctctcctgtcgggatcatatagggctgcagtgacttcaagagtcctactggcccagcccctgctctaggcagttctcataattgcaaaaatacctctgcgtattgagatttactctgtagcagacattgggctgggctcttcttcctggccgttgaatgaaccaaattcagtcctgactcacagcctttgtactccctgttccctttgcatgacatgccttttcctcagaatttttctaggctcatttcttcttgtcaaaaggtatttcctataatgtcacctcctctgagaaggctctctgaccactcaagctagaggaggcccccttaccctcagtctaacacatcatcttgtttttatttcttatatatttgtttacccatttataatctagagtctagacattagactacaagttccttgagggtggagatcttttcttttctgttcacaatgtatacccaacagctagcagagtgcctggcacatagtaggtgctcagttataaatatttgtggaataactgaatatacaagtgaatgatattactagctttataataattccatgaatttgatgctatgtttctattctgataatggggatgttgaggctcagagaggctaaaaaacatcccaagttcacactgctagtcagaggcagaacttgaaccccagcttctgacactggagtccttaattgctctgttgtgttgcattctcagcatttagcacagagcctggcacatacttgaagcttaatataggtttgcatgaatgaatgagtgcatgaatgcataatttaataaatggcatcctatgtgtccaatacgtctctctggctgggattaactcagaatcaactcttggaaaggagttttcttgtttgactctttctgaagcttttttcttggtgggcaggtggggggtcagtgggaggccaaaggtgcaaaagtgaagcccccagcgccagacaaggtagttcacacctgtaattccagcactttgggaggctgaggcaggaggattacttgaggccagtagttcgagaccagcctgggcaacatagtgagactctataaaataataataataataataataataataataataataataaggaaaaaaattagctgagcaagatggcacatgcctgcagtcctagctacttgggaggctgaggcaggaggatcgcttgggcccaggagttaaaggctgcagtgagttatgattgtgccactgcactgcactccagcatgagcaacagagcaagaccaagtcactaaaaaaaaaataataataaaataaaaataaggaaggccccagttaaattgtgtctacctttttgccagatacgagtggtttcgggaactggagctaaagtggtacgccctgcctgcagtggccaacatgctgcttgaggtgggcggcctggagttcccagggtgccccttcaatggctggtacatgggcacagagatcggagtccgggacttctgtgacgtccagcgctacaacatcctggaggtaacccagccagcccagctggccagatgaaggtgtgggcctgggggtggcttcagggaccgactcgccgctatcactctgcctctgtaggaagtgggcaggagaatgggcctggaaacgcacaagctggcctcgctctggaaagaccaggctgtcgttgagatcaacattgctgtgctccatagtttccaggtatgtctggagttttgtgagtctgaagactttttggtgagaagcttaactgatccagtgacttatgtgatgcttaatttataagtaagcccccttgctcactctagaggtccagcaacaggcagctcctcacctcccaagactgttcaatccctgtacttgagcactgaattctgtcctctctcaatcgctcaaggactttactcccaaaaattttcctcttctctctctgtatcatcaatgtctctctctctgctgagtcattcccagcagtgtacagacaggctctcttagccccatttcaaagaaagaaatgaatagctaaccacataactcctgccaaacctcaggctccctccagccactgcctcacccttctgctcctttctacagcaaaactatggagagatctcattgtgcttcctcatcccccaactgcttcagcctggctgggcgacagagcaagactccgctcaaaaaaaaaagaaggcctccctgaccgtgccatgtaaaatgacgccctcaccccactgccaacctgtctctctgtgttagtcctttaaccattttatttttcttcaaaatacttcctcatacagttcctggcattatataggacatcgactttcttctttcttttcctttttttttttttgagacatcatctcactcggtcacccaggctggaatgcagtggcatgatcactgcagccttgacctcctgggctcaagtgatcctcccaccttagcctgccgagtagctgggactacaggtgcataccacgcttgcctaatttttatatttttagtagagatggtgtttttccatgttgcccaggttggttccgaactcctggcctcaagtgatccgcttgcctcagcctcccaaagcgctgggattataggcataagccacggtgccgggccagctttctttattgtttggtttcccccactagaatgtaagctccatggggcagggaccttgtctgtctgtttgctgctgcacctctggcctagaagagtgtctggcacatggtagatgctaaaataatttgtctttgaagaatgaatgatgagggccacatccgcgatctggcctggcacaaactgggtcttcagtccttgtctgacgtgtgcatgaatgagtgatgctgggaggggacctgtgagctgtggctgctcttgcttgctgtgcgaggataacaacctccacctcctcttaagcccacacacagccgctgggctcctgtgtagggctaagccctcaggcccatccgctgctcttactcatagaaacacttcccttccctgtgggaaggaggcctgagatgtaggaagatactactttagaagccacagccattgccacaaagctgacagatgccctggagaccccaccactggtcagtcattcattcattcagcagtgttttactcagcatttgcctggtgctgggcactaggctgggccaaggatgtgagtggtgaacagagtacagatgagcacacactgttccccatgactgggtcaccccataggcagatcaggcctgtgcttgacgggggaggggaagatggaacaggaagagccccccggtgtttggggaagggctggctttgcaccacacatcctgactcctggggactcccagcaccagccctgatgctggcaggatgtggatacgtcccagaaaatctgtgagcctctgctgtctcctcaccgtggctgactgtgtccttccactgcctgagcgtctccaggacttcctcaccaccctccttcagggtaaacttcaaaagatgggcccttgtttcctcctctgtaaaatggggataatagtgcctccctcatctgaggattataagagttaagtaaaaatgcatgcaaaatgtttagctgcactgcctggcccatagacaacactcagtaagcaatgattttcttactgtttattatgttgtcatcatgatcgtggctaccactgagttttttttttttttttttttttcctttggagatgaactctccctccattgcctaggctggagtgcatggtgtgatctcggctcactgcaacctccgcctcctgggttcaagcaattctcctgcctcagcctcccaagtagctgggattacaggattgcaccaccatacccggctaatttttgtaattttagtagagatagggtttcaccatgttggccaggctggtctcaaactcctggcctcaagtgatccacctgtctctgcctcccaaagtgctgggattacaggtgtgagccactgcacccatcctgtagctaccactgagcttagaactgatctggctcccaggagttgctgtgtcttggaagaataagtgaatgagagcccacgtccacaagagtggtggccggacttttatatacacatatataacttatgtatgtctattgaatttacaaggcatggctggccttcccaggcctgacaccagctggatcctctcatgcctccctgccttgactcatgcagtgccccactcaactaacttgcacccctctcacaagtctcccttcctctcaggagctgctcccgaaattcgccaactcaccctgggcctctgtctggcgttgtatcctctcttctgcccttccccagcacctttgcctgcctccatcagagtttctatcaggccgtgtgaaaatgactgattttccttggcttccccacccagttagactagactctaatgcagagaaagggctgtgtctttcccttctataacgccgatgccttatgcagtgcctggcagatggagattataagagcagctaatatttattaagcacttactatgctttgcttattccagcttattgcatcctttcaacaacctcatgatgtaagaactttttttttggagacaaggtcttgttttgtcacccagactggagtgcagtggcccaatcatggctcactgcagccttgacctcctgggctcaagcaatgctcttaccacatcagcctcccacatatctgagaacactggcatgtgccaccaaacctggctaatttttaaatttttttgtttttgttttttttaagacagagtctcgctctgtcacccaggctggagtgcagtggtgcgatctcggctcactgcaacctccgcctcccgggttcaagcgattgtccttctttagccttctgagtagctgggattacaggtgtgtgcctccaccatgcctggctaattattatattttttagtagagacggggtttcaccatgttggtcaggctggtctcaaactcccgacctcaggtgatctccccgtctcgccctcccaaagtgctgggattataggtgtaagccccgacagctggcctactaatttttaaatttttatagagacagggtctcaccatgtttcctaggctgatttcaaactcctgggctcaagcagtcctcccacctcggcctcccaaagtgctaggattacaggcatgagccactgcacctggccattataggaattcatatcactcccatttcacagatgagaaaacttaaagaactaattgctcaaggtcacatggcaaataaaaagccagagtttgaaccactgtgttaaacaaatgtctcttgactgaatgggttgccacagacatagaggccagtggctgccttgctggaccattccatggggtcagaccccacagcccgtaatttcagactgtgagtgattcgtctcccatacactacagttctcccaatgcttcagacctccccacggaggggtctggctccttcttggctgaggaggctacaaccccatggaagtgactcttagaactctccaccataaatggctgggatggtatttcctttacctgccaaaaggtgctgtggtttaagttatgcagatccagagagagccatttgctgggagtctggggagacctgtgggcatgagaggctggatattgccctcaaagacttaagggaggcccccatgggacctgtgggctgaccctgactaatgtgctttcttgggctctgtcagaggctccacaagcgccttccatttgggtgtttattttcagcaggcaaggagagggccaggagttgctctcccgtgttacagattgccaggccttcccaggcactctctgggagccctgcggccaaccggcccctctcctgattcccccacccttatctccactccctatcttgccttctgatacagaagcagaatgtgaccatcatggaccaccactcggctgcagaatccttcatgaagtacatgcagaatgaataccggtcccgtgggggctgcccggcagactggatttggctggtccctcccatgtctgggagcatcacccccgtgtttcaccaggagatgctgaactacgtcctgtcccctttctactactatcaggttaggggccttcctcttctctccttcctgggggctcagatgtggctgtatgtgtgtgtgtgtgtgtgcacctcattgttacacatgtagggcacgacgggaagcaggagtatgttgttagagctttccaggccccactcaaggatgctgagcagcctttgtgcatttccacggagagtggcactgaaggaagtggcttccactgcaccgctagtggcctgtggctcagatggggggtctgtcattacacctgcttccacgggagggcccggcaacagtcactcagcagagtgttgaggggattggcccctgagtcagacgctattgattctgccacttcctagcaatagggtcttgggtgggtcctttcacctttctgagccctgatcttctcatctctaaaagggatgggcttcactccacgacggggggttgtgtggatcacaaagctggtgtatggaacagctcagcccagtgcccaatgcagaatgcattcagtaaccatgagctattgagaagagttcattttgtgccaaaaaggacttgggttggattttaagagcttccttcatctaaaggaagagcttcctccatctacaggacctcacatgcagggtgacggcatgtcctggagtcttgcctgttgtagtagcagctcttcttgctctcaaaggtgtctcggttgggactgtacattatgtcatctgcctgctgataagatgctaaaagcaaaaggagtgcagcctgttccttccaggaggacactaatgagaataagggttagccgccccccctcgatgtgcaggctccatgccgggcagtggagaggcaactctgggggagaggcagccacggccctcccttgtgggagttcagtgacaggggtggggcgcaggccagccttctctcccaggctcatgatgggtgtgaagggtcctctaccactccagcatccgcgagctgagccaagccaggaccccgccctgccctcacatctcctttcttttctgaaggtagaggcctggaaaacccatgtctggcaggacgagaagcggagacccaagagaagagagattccattgaaagtcttggtcaagtgagtggcagtggcagtggctagtagtgatgtgtgaactatatctctcccttctagtcagcctctgcctccagtccttgaagggcaagaacacaaggaatggttatgctttttgcttttgtttttgagacagggtcttactctgtcacccaggctggggtgtagtggtgccatcacagctcactgcagcctctgcttcccgagctcaagtgatcctcccacctgagcctcccaagtagctgggaccacaggtacaaacccacgcctgactaattttgtttgttttttttgtagagacggggtctcgcagtgttgcccaggttggtcttgaactcctgggctcaagcgatcttcctgctcagcctcccaaagtgctgggattacagatgtgagccacggcacccagcctgggaatgtatattcttcgtggaatttggctcccagaggtccagtcgccaattccaagggtcccctgctgccattgttccaaccttctctctcctcccttttcccaaaggggcctatagtagtgggtgaggggaggaaaagggtgagttgtcacggtacatggctggtgagaaacgtagcttgcttttccctcaacctgccccacattaaacgctgtcccctcccattcccctggtcccccattttcttctggaaggctgactcagcacacagcgcctgcctgtctgtctgtccccacagagctgtgctctttgcctgtatgctgatgcgcaagacaatggcgtcccgagtcagagtcaccatcctctttgcgacagagacaggaaaatcagaggcgctggcctgggacctgggggccttattcagctgtgccttcaaccccaaggtactggctcagtgggatggctgttgttttttctgcttgtgtgcaggggagtctaggaaaagttaaaacggtttccccactcctggatgtctcagagtcttggatgtggtaatgtgcatcattaatctggaagagaaggaaagacttgtgtgcagggattccaaaacttacacagtagatgagtttttcatggtgttctggagaacacagtttgagaaacactttcacaatgaatccaggcctcaacatacatcagctgctctggctgaactttattcaggctcactgaacacctgctctgcaggatgcagtgctgcatgtgaaagggggaagagatgagtaagcttcagataaacaaaaaaaggccgcagagggtctgtctaagctctatcccctgccttcagcactgatggataaaatccaactcttagggaatggtggccacgtgctggaccagccccgggctctgaggatctgacagtgggtggcgcagagccaggccttgcccttggggagctctccagcatacaccccaccctcccctcccagcgccctgcaaagcaggcgtcaatgccattgttaatgcacagaggaggaacctgactgttagacaggttgatgggttttccagggtcgcacggcttctgggagatggatgtgactctgaggacagggcacaggccagtgtaatgccacgatgggatgagctgtggtctgtgccacgtagaggcttaggccaagggggcactgactgatgaccaggtcggccaggttgctgaaaacacctcttgggtccaagcctcacctgctggctcccaggagtctggaactgccaggagagtggtggcaggtcccccatcctccgctgggtgggcctggatagaacagcaagatgagggcacatttccccggccattccctccaggcacagctgtgacctgctcattccaattttgtggaaatatttccacacacacaaaattgcaaatagtagtggacatggtgagaggcatttagacatgggaaaggcagtatttttgggacagatcttccagggcttgccaatgtgttaaccatagggcttaagcggggaaggagaacagaggatgatgtgttcaaattggttcattcacgtcctctgtgccacgcctgtgccgggccctgggagagacagatgagcacaggagccgcagcctgggggcagtgtgggaggaagcccagggtgtctgcgcagggtagggagggccctactgggagccgagggctgatgtccacttgggtgccatccaggttgtctgcatggataagtacaggctgagctgcctggaggaggaacggctgctgttggtggtgaccagtacgtttggcaatggagactgccctggcaatggagaggtgggtggcttgaggtctggctggtctagggggtggtagggcacgggaggacccaggaaagggtctgggggatgcggcaggtcctacggaggacatttgggagtcagtgctcaggccactccgggtcactcaggtcatttgccggcctccattgtaattattgccatatgggagtgccacctttcctatgacatatttaatatatttctgtgaatggcctacctgtttgtatttacaaattcatatttaaaggaaacttgtatcactctcacaaatggaaagcagcaaaacataaattcaatgaaaacaaaatgaagttaatgaattttagctagatactattccctggccaaggcctgctctttgtttgttttgtttgttttttggtttttggtgtttgtttgtttatttatttatttatttacttaggtggagttttgcttttgttgcccagggtagagtgcaatggtgcgatctcagctcactgcaaactctgcctccctggttcaagtgattcttctgcctcagcctcccgagtagctgcgattacaggcacgcgccaccatgcctggccaattttgtattcttttttttaagtagagatggggtttctccatgttggtcaggctggtcttgaactcccaacctcaggtgatccgcccgcctcagcctcccaaagtgctgggattataggcgtgagccaccgtgctcagctatctttgtttttaaaaaagactaataggcaatgagagaggtattaaaggtctattagcaccaatatgagactgtctctccagtgctccgaagaatgaaaatgtaattgaaggggaataactaaaaaataaattatgaaaagggagtaaatactcaccatgggatttgacgctagttctgctgggtcacatacctcctggaatcatcttggcttcctggaaagccagccttatttcagagactgtaggaccaattgttgttgcttctaccgggttccaaacagaatcacggcgcattcggagcccctctggagaaggcatgctaccaggcaggcccctctgccagggctggatgggtgggggcccagctgttccaggtcccccattaagaggcagggcctacacaagttgctacagtttagttggagtgagagacaagctaaacttgtcagctgacaaccaggtccttggccaggtagccccatatgtaaaccaacttccgtggtgggctgtgagccttctcctgcaagctgtggccaggtttccagaagaaaggaaaaaacagtgtttttattcatcctggcttgagaactctgtcattccctctctttctagaaactgaagaaatcgctcttcatgctgaaagagctcaacaacaaattcaggtaaagctttttccgttgtcctactcctagccctgccctgtggacccaaagtctgggttcaggggatggaactgggtgtgcacagggggtcttctcagaaccgtgcaggcacagagacctgggtctaaggtctgtactcagaatgtgtcgagggtcaaaccacagaaaccaagtccctctcacaggtctgcaggccaagcatggggtggtcatgggagttggtgacaaggctgggggatggagaggaccagaagggggtccagctccattctccagctggagggggagagtctggggctggctctccaggctgttggtgctgggtgctgggtgccaggtgctgcttgcatgggacatgtgtctctgtgtgtgtcctcgggcaggtacgctgtgtttggcctcggctccagcatgtaccctcggttctgcgcctttgctcatgacattgatcagaagctgtcccacctgggggcctctcagctcaccccgatgggagaaggggatgagctcagtgggcaggaggacgccttccgcagctgggccgtgcaaaccttcaaggtcagttcccagccagagctgccccacagcagggacatgacagggtgcattcatgcctaggacatctgtgctgggcgtcccaggtggcatgacctctgcctgtggtcctgtagatgtggtcccaggcatgggccagggccacaaacctgagggcttaggtcactgctgctctccctcagccccctagaagagctccaccttgtgcacacaactctgggggagaaggtggcacagatacccccctgcccttagggagcttctagtttaaggtctttcctccagtgactacctaggtcacctcttaactactgtgtctgtttcagtctcacagccatagagagcctggggacaggcaccacacaatggcggatccactttggatccactcagtatctccatagctctttattgctgtgattgccccagtcatttagggaactcgtcactcatgcagagcgtcactcagcggtggtgcttaggagcatcagcttgcatcggggcaggcttggttccaaagcccaggcctcctccatgtggctccacaccaggaacataagagcaatcagacctcttccagtgtgggcatgaggatacaacaagatcatgtcaatattggcattcatagtggtcgggcacagtggagcatgcctgcaatccaagcactttaggaggctgaggtgggcagattgcttgagcccaggagcttgaaactagcctgagtaacatgtgaaacctgcctctaccgaaaatacagaaaattggctgggcttggttgtgcgtacctgtagtctcagctacaggtacgggactgaggcgggaggattgcttgagcccaggtggttggggcttcagtgagctatgatggcaccactgtactccagcctgggcgactgagtgagaccctgtctcaaaacaaaaacaaagcaatatttataatagtagcaatagctactatgtgccaagcccagacacctctttgagtccttgctgtcaccctatcaggtaagtgtgcttcgaagttacacgaaacacacggctcagtgtttggcccatggtaagggcctaaaaagtggtagccccagtggtggggatgctgctgctgctgaccattaaccgcagtctgctccaccttcttccaggcagcctgtgagacgtttgatgtccgaggcaaacagcacattcagatccccaagctctacacctccaatgtgacctgggacccgcaccactacaggctcgtgcaggactcacagcctttggacctcagcaaaggtacctgcttctctgcagcacatggggcttgtttgtgttggggtctgagccctaagcccatggtcaaggagacccccaggtctttctgaacagagacagctggcctcggggcctccctctcactgcatgcaagagcctgttagggcataatactcaaggtgctgagggaggctgtttcaggagggagccccaggagggtggtggagacagaagggatcagcatccgccaagggcctactgtgtgtcagacactgtgcggggctcctggcccacatgggctaagtgagcctggacactcctcctgggagaaaggcacagatggagaaattgcagttcaaggaggtgaagcaagctgctagcctgtggccatgttggggtctgagccccagccttccagctgcgaaggcagccaagtgtcgtagagaaggtgtcacagaggcgattccaggctgtagtggtgaactctccactctgcacccccaggtgctgtgccctgtgccctgtctaagatagtcctgaaggtttctgcatatttaagatatccccaatgtcaacaatgctctcctgtggatcccaagccatggagatgtcccgggactttccatttttaggttcctaaattgaatttcccaacacctagaagcaacacttagaagctgccctgtatcagatcaaggagcttttttttttttttttttttttttgagacggagtctcggtctgtcgtccaggctggagtgcagtggcgcgattcccgcttactgcaacctccgtctcctggcttcaagtgattctcgtgtagctgggactattggcactaccaccatgcctggtttcagtatgttggccagtctggtctcaaactcttgacctccagtaatccacccaccgcggcctccaaaagtgctgggattacaggcatgagccaccatgcccggcctggaccaaggatctttatttgtggaataaaaggaagaaaaagaaaaacccaacggccacctcagcaggatgccccaagggtggtgttcagatgtcactgactcagacatctgggggcttcccctacctacgctcaagaaccactttgccatttcaccgtctctgtgtcctccacagccctcagcagcatgcatgccaagaacgtgttcaccatgaggctcaaatctcggcagaatctacaaagtccgacatccaggtaagggaaagtgcagggcttcgtgggtgatactctgtcgattttattttaacgaatgaaagatcagaagaagtcagtctttgaagggagaggagaagagcatatgctggcattagcagccacactgtcggtaggaccggcccacctggtaacctgcggccacctgtgcttttacgtctactcttggttaaccacgagccacttttccagcttggactctaagcatctgctccacttcctctccttcctcattgaactctttcactaaaaggagagtgcaagagagacttaaactgtttgattcattcttaagaactttcaggaaaagtgttggcagggaaggaaatttcccagctctgggaaagagccttgtggattatctgcaggtttcattgatctgtgctgtcctccccgcatgcattaggaaaactggccttggttctaataagaacagggtttgtcctggcaacaaggaaaggtttcttctgatatccatgtatgtccaaggcggatgctttctttaggcagaggctgtagccaagccctcggggggctccaagggctgctgggaaggggtgggcccccctcctccccagagggaagctcctggggccctcttaagcacccctgcctgtggggatgcctcatcccaagctcagggtggtggcctagcttgtggaaccagccaggaaaatgaaaggagggcacatttcctggggaatctcgcctgcccatcaaggagccctggaggaaatcctgcaatgtcacctgaggcttcattttccttccccagccgtgccaccatcctggtggaactctcctgtgaggatggccaaggcctgaactacctgccgggggagcaccttggggtttgcccaggcaaccagccggccctggtccaaggtatcctggagcgagtggtggatggccccacaccccaccagacagtgcgcctggaggccctggatgagagtggtgagccccgggctagaaagccagtgctgcctggcaccctctggccgccccacctggctgaggggctgagcctgggcctgccggtgacagccatccctgtccttcctgctgagagggcgggccaccaaagcttatgtgaaagcagctgtggaaacagtcccagcccataaggcatggccccctggccttagtttccctaatgataacatccccctcaatgcagtggccaaagtcagaaagtgggatgagctgggtgtggtggctcacgcctgtaatctcagcactttgggaggccgtggtgggtggatcacttgaggtcaggagtctgagacaatcctggccaacatggcaaaaccccgtctctactaaaaatacaaaaataattagacaggaatggtggtacatgcctgttatcccaatcccagccactcgggaggctgaggcacaagaatcgcttgaagccaggaggtggaggttgcagtgatatgagatctcgccactacactccagcctgggtgacagagactccatctcaaaaaaataaaaaagaaagtgggatcagagaaacagagcctggcagtgacccccactgtccaggagaaagtgagagaacctgagacgtggggacgacaagacagttgcccttgtgactgtagggtgaaggatgtggttcatcaggactctgttccagaggagagaatgtcaggctcttatttttacccagtgtttacaaagcatctgtgtaccaggcacggcaccaactattatggcacaatttcattcagtgctcatgccaacccagcaagataggttgtgctatcagatctgtttcacagggagggaagccaaggcccagaggcttcagtcacctcagaagtggggagccagggtttgaacccgtgtcttctggcaccagggtctgtgctgtgactgccctgcatgctgctccctgggacttccctttgggctcatctgctcacccagcacatcttggtacagcacccaggggactgcagtcatgggcttctctctaagtgaacaactggcctgtgaggtatccattagtctttggtggatttagtcattgtgagtctgtagatcaccagtgagtgtcttgctaaagttcaggtctgggccagagaaggacgtggttagtttatcaggaccacgccaacttactgcccctttgggggctggtgatcagccttccctggcagtcctttctccatgcgccaccttcttcctgccttttgctcttagagccaggtctttagagcctcctttcccctgcagctggagaatagagggtgtctgcctgggggcctcgctacctgctgagaatcctgaatggggcctcttttccatcctgacaccttccttccccatccaggcagctactgggtcagtgacaagaggctgcccccctgctcactcagccaggccctcacctacttcctggacatcaccacacccccaacccagctgctgctccaaaagctggcccaggtggccacagaagagcctgagagacagaggctggaggccctgtgccaggtgaagatcagggctggtgggcagtgggggtatgtggggggtcccagcatgtggggagccagagtccagagctggttttaactttcaagagggatccacaccaggttcccaaagtgagacttgtcaaaggctggtgggaagccaggcatcgtccgctggcccatgtctaggacacagttgtgttgagtgaactgaatttttccaagtgtctgcaggatgcaaccagagtgcactgccttgaacattagctagaaaggggagccgcccttatccgtctttgctttccccaaaactggaataaacttgtcacttaagatgtattgggggatggatgatgtggatgaatggataaatgggcataatgggcagagtgacagatggagagctgaattaataagttaatgagagaatgcgttaactcacctgcaactgtctggttccaagtctttgcaaaaggcactggttgtcattattatcatcatcattatagctaattatctcaatttaagctacctccaccgccattattttgtggctgcaatgtactaacttggcacatgctttctcatttaattcccataacatccccacaagacaggcaatactgtccttgtttcacaaagggtaattgggacaagattccacagctagtgaggacacccagccaagatttgaacccaggtctgattggttccaaagccttgccccaaataaattacaataaagatttgacccctgcccatgagttgccgacagtggaggtaacacaagttagataaatgtactggactgagttcctccaggacaggcacttattttgtctcaagtttggtcgtctcagcaccatggacagtgcctggcacatggacttgtttgttgagtgagtgaatgaatgaatgaatactgggttgtgacagtcttttccccaaagcgggacaggcgccctgagtcactgacatcacccttggcttttagccctcagagtacagcaagtggaagttcaccaacagccccacattcctggaggtgctagaggagttcccgtccctgcgggtgtctgctggcttcctgctttcccagctccccattctgaagcccaggttctactccatcagctcctcccgggatcacacgcccacagagatccacctgactgtggccgtggtcacctaccacacccgaggtgagccaggggcagaggctctggaacagccccgctccgcccattattggacatctgttcatcaactcagattcagctccctcatcagttcaaggagggagggaacacccacagcccagagttgttaacagtattaggaggtgtgtgggagagatctctaggaaccaggcactaaaccaagccttctgtatgtatatgatcagttacattgtggggtaggcactgtgtgcattttacaggtggcacagaaagtcacactgttagtaagtggagagctgagattcagacctggatagtctaactgctgagtccaagtcctccaccactccactgtcccactactcttgggacaatttaatgagatcagatgtgaagagcactcaacccttggtaggaggccagtggttcttagtcttcccaatcatgcagcctgaacgcaggggagattctgcattgattattcattcactcactcagctattcagtgggtgcctggcactggggtgggtatggaaatacaacagagatcagccctgccctcagggagcatctagaggcggcagtggagagataggcatagaaataattagatgattttcattgtgctgaagccaagaaggaaaggtcagggtcactgagggaagcagagcagggggtgacgctgccatagagcgggaggccaacatctcgtccaggtggtctcggctccaccactggccgctgccccctgccctgccctctgtaactgagcattgcctgggttggcaaagctgggagctgcccacacttccgagccctctgtcctcaagaagcaaaaacacagtttctaaaaccttggcagagactctatttgccctgctagagaacagccacccagaccaggagaggagaggcagacgtgtcctgtgagatcaaagtttaatcccgaggaaaaccagggggacctctgcagttggttggaggccttcctcaagtgctgggccattgtaaagggaacacaacccagagagacctcctgggtttctacaagcccaggagggcccactgaggaggaaagcaaggtgccagagcaggctgaaatgcgcggaactcatccaccacccagggttgctaccagcaagggccacgctcatgagcacgcacagcagtagccaccctgaggggccacttcatgctgctgtcagtggagctcagtggtgagctgggagtgttcccggagaaagtctttggaaggagcactggagtaggagtcagggaagctgagctgtccacctgattccaccacgacctgagtgacctttttcaagtcacttttcctctccccgtctcagtttctgcttgggcagaaggagcaacttgcagcctctctaagtccctttcaacgttcctcagctccgcgcttacagagggtggatctcagaaccaggaggacaaacagctgctcattgagtcctgagagtgcctgagtgggggcgtccaaggagggccgtggtgtgggcggagcagctggtacagtttgcgcatgggcagtggggggacgttggtcactttgtggtatcgttgcagatggccagggtcccctgcaccacggcgtctgcagcacatggctcaacagcctgaagccccaagacccagtgccctgctttgtgcggaagtaagcaccccttccactctgtcccctgtggggccgccgacccctgggagttccgggaggaaagggagggatctggggtttaggagacgctcggagccccttggcccataaagaaggcactggggtcacactggtgtggctgcctgtagcagggacagcgatgacaggtgggtctcaagagtccagtgagcttggcccttttacagaaggacctgggggaggtgctggtgcctggacagaagtgaagcagcctgcaggacgtgggaaatcaaataggagcttcttgtcctctccaccccctcacgaccgcctcccacaggcccagctccccagccatcctgtgtgtgcgtgggcctggggtgtctgaggactggccccctcttctcctgtcctcttcagtgccagcggcttccacctccccgaggatccctcccatccttgcatcctcatcgggcctggcacaggcatcgcgcccttccgcagtttctggcagcaacggctccatgactcccagcacaagggtaaggctggaggcttcccaggtgggaggggcaccagggggccagcgcctgcctaggagcagaactgcagccccacaaacggttcctgacctctctcgagggaaggcctcccaagtttgcttccctgtgcgggcctctgcgggttagaatggctgcctttccgtctcccgcatagtagccagggctccctctcctgggagtggaggtaacaggaaagtccctgcttttgatctccgattggctgagggaattctgcatccgcactttcagttctgtgactatggatgagtgatctgaatgcgctgggcctcagtttcctcatctgtaaagtggggataatgctattaaatagctagtgggagtgaagtgagaattccagttgggcttggagccctggcgccctctggtggccaacatggtaacaacagcccgggactcctgcaggagtgcggggaggccgcatgaccttggtgtttgggtgccgccgcccagatgaggaccacatctaccaggaggagatgctggagatggcccagaagggggtgctgcatgcggtgcacacagcctattcccgcctgcctggcaagcccaaggtaaatgcagcctcaaagcaggtgaagcattacacacacaagctccactgtgagccctgggtccccacaggcccctcgcctccccgagccttcacctcctgagctcgaaaatgctggtaattcacaaccatcagagtgatgctgtgaagagcattacaccctctcatacagcagaagcgacagacctggagaggttcaggactaacctaggtcacacgcgggttaaccccagagcccagcctgctgaaggtccccagcaggccccctgggacacgcgactgcccggggagaggtggtccaggctggctacgctcaagccttgcaaggctggagggcggccgatgcttccaccacggccatcagaggggctcttctgtccccagggccgcgttaccccctgagcctggggtgtcagaatagactctcggccttcacacatttgtcccatcagggcacctcttgggcccagtgctctgtgcccagtgctggggccactgggcatcctgtgtggagcaggagcagagagtccctgactgactcctggaccttgtatctggggagcccgggatccataccctgcagagacctaatgggtagactgcattgagaggctcccttttctggggtgtaggcagagccctgggcagggggactgccttcattgatgcctttttgaagcatggcaaattcagagacagtctgggatccccactggatggtcagcccctcgagggcaggactgtgtccagcatttatctctctaatcccagccgagccccaggggcatccatgcacctcatcctgagcctggccccttgcaaggtcctggctggaaggggactcactcctcaccccttagtgcccctctccccgcctccttgaggccagcaggcctgcctgcaagccttgtcctgagcagctgaggactgaacccactgtttcctctggtttcaggtctatgttcaggacatcctgcggcagcagctggccagcgaggtgctccgtgtgctccacaaggagccaggccacctctatgtttgcggggatgtgcgcatggcccgggacgtggcccacaccctgaagcagctggtggctgccaagctgaaattgaatgaggagcaggtcgaggactatttctttcagctcaaggtatagtagtgggtgtgtgggctgaaggtgccggccaagggcacaggccgttggagccaggaccaggggtggcaggtagacccaggggaatcaggcccagaaaggttctggaccccaagagaatcttagcagtggctgagatctgagctgggtgggctgtcagatggccttactgcacatcctggaccagttggcagctttgagaagtcgggtgaatgtgttccatttttccatctgtagaatgggggcaggagagtctccctggcctctccccaaacccccctttcagaaagtatctgtgtggccaaagcaaggctgtgaacacaggggagagcaaggctgccccccgtcagcctcgtgtgaacctcacacttcttatttacacccaggctggtgtctgctcaccctcatccccccagcaggaagggcttgcattttattgatgaagataatagtgatatttatcaagggcttacactgggcatactgcttgccaaggattctcatgtgacatttcattcagtcctgccgaggactttatgagggaggcacccccttttacagagggaagggggcctacggggaccacagcagtaaatgtgagagcctagagttcagtctgcctctctctggttccaaagcctgtgattgtcacttccatactctccctaagacacattcatacgttcaactgaacatttattgagcacctactatgtgctagggatagagcagtaaatgaatcaggcaaaaattctttacattctactggggctgacaaacccagacaagaaagaggtgtagtgcttcagagaaaaacagcaggaaacagcccagggaacgctggggagtgtaattttaaatagggtggccagggaaggcagagctggaaggtggcatctgagaataaacctgagatggtgaggaggagccctgtggatactgaaggaaacactctgggctgttctggaggggcagcaggtacaaaggccccgggggtgggagttaagctggcgtgtttgaagaacagtgaggagataacaggcatttccccaaatgtgttcagtgggtcggtagacagttggcatcaaaaagattccaaggtcgaatagcctgggacatgcttagttaaggcaaggcaaacacgttgctttactacaagacttctgggagccttcagtatgctgctgttcatcaggacccttcaaggctggaaggaaagaaaggacgtagcatttcccaaacatatttatctcagagctcacacttcctggagtatcttgggtgggggatgaacgctgggcaggaaatgggtatgctaagtaaagctttggagatggttagagggcccgcaccagagcaggtgctgagcagggttagggtctaaaccagattcaagcacaatctaagtataacagttgggaagacagatggctcatgaaggtccatgtgatagggaagctgggcaaccccaggggctcatcccagcctggacttgacacgttgctctctgtttttacctaaaagagccagaagcgctatcacgaagatatctttggtgctgtatttccttacgaggcgaagaaggacagggtggcggtgcagcccagcagcctggagatgtcagcgctctgagggcctacaggaggggttaaagctgccggcacagaacttaaggatggagccagctctgcattatctgaggtcacagggcctggggagatggaggaaagtgatatcccccagcctcaagtcttatttcctcaacgttgctccccatcaagccctttacttgacctcctaacaagtagcaccctggattgatcggagcctcctctctcaaactggggcctccctggtcccttggagacaaaatcttaaatgccaggcctggcaagtgggtgaaagatggaacttgctgctgagtgcaccacttcaagtgaccaccaggaggtgctatcgcaccactgtgtatttaactgccttgtgtacagttatttatgcctctgtatttaaaaaactaacacccagtctgttccccatggccacttgggtcttccctgtatgattccttgatggagatatttacatgaattgcattttactttaatcac

SEQ ID NO: 6 - DNA, 129 bp, Homo sapiens
gtacccggcccagcctcagccgccggccattggggcggggagccccgtggtgagcgagtgacagagtggagcccagaggagacacgcagcccgggcttacagactcacagggcccgtcttgttccccag

(19) United States
(12) Patent Application Publication
(10) Pub. No.: US 2010/0216121 A1
(43) Pub. Date: Aug. 26, 2010

(54) Method for the detection of predisposition to high altitude pulmonary edema

(75) Inventors: Abdul Qadar Mohammad, New Delhi (IN); Aarif Ahsan, New Delhi (IN)

Correspondence Address: DRINKER BIDDLE & REATH;ATTN: INTELLECTUAL PROPERTY GROUP, ONE LOGAN SQUARE, SUITE 2000, PHILADELPHIA, PA, 19103-6996, US

(73) Assignee: Council of Scientific and Industrial Research, New Delhi (IN)

(21) Appl. No.: 12/222,495

(22) Filed: Aug. 11, 2008

Related U.S. Application Data
(62) Division of application No. 11/299,541, filed on Dec. 12, 2005, now abandoned.
Application No. 11/299,541 is a continuation-in-part of application No. 10/713,137, filed on Nov. 13, 2003, now abandoned.

(30) Foreign Application Priority Data
Nov. 14, 2003 (IB) PCT/IB2003/005158
Nov. 24, 2003 (IN) 1455/DEL/2003

(51) Int. Cl. C12Q 1/68

(52) U.S. Cl. 435/6

(57) ABSTRACT

The present invention encompasses methods, compositions and kits for the detection of susceptibility to high altitude pulmonary edema in a human subject. The present invention further encompasses isolated polynucleotides for the detection of susceptibility to high altitude pulmonary edema in a human subject.

### CROSS-REFERENCE TO RELATED APPLICATIONS

This application is a continuation-in-part of U.S. patent application Ser. No. 10/713,137, filed on Nov. 13, 2003, which is hereby incorporated by reference in its entirety herein.

### FIELD OF INVENTION

The present invention relates to a method for the detection of predisposition to high altitude pulmonary edema (HAPE). It particularly relates to the allelic variants of iNOS (inducible nitric oxide synthase) gene, which has been found to be related to the prevalence of HAPE.

### BACKGROUND OF THE WORK

High altitude pulmonary edema (HAPE) is a form of noncardiogenic pulmonary edema that develops in approximately 10% of randomly selected mountaineers within 24 hours after rapid ascent to an altitude above 4,000 m. A similar phenomenon is observed in the lowlander inductees ascending to a height above 3000 m. An even higher incidence rate of about 60% has been demonstrated in subjects who are susceptible to HAPE as documented by a previous occurrence of the disease (Houston, 1960, N. Engl. J. Med., 263:478-480; Bartsch, 1997, Respiration, 64: 435-443; Bartsch, et al., 1990, Hypoxia: The Adaptations, 241-245). HAPE can be effectively prevented by prophylactic use of vasodilators or slow ascent. Nevertheless, it remains the most common cause of death related to high altitude exposure during trekking or mountaineering (Hackett, et al., 1990, J. Wilderness Med., 1: 3-26). The morbidity rate in Himalayan mountaineers was estimated to be 50% if immediate treatment with supplemental oxygen or rapid descent was impossible (Lobenhoffer, et al., High Altitude Physiology and Medicine, Springer-Verlag, New York, N.Y., 1982: 219-231.). Observed differences in clinical presentations and severity of the disease between racial and ethnic groups together with familial clustering favor a significant hereditary predisposition to the disease.

Although knowledge of the factors influencing the development of HAPE is still incomplete, there is experimental evidence that an exaggerated hypoxic pulmonary vasoconstriction (HPV) plays an important role (Scherrer, et al., 1996, N. Engl. J. Med., 334: 624-629). An excessive rise in pulmonary artery pressure has been demonstrated by invasive and noninvasive measurements at high altitude in individuals with HAPE. The uneven vasoconstriction in the capillaries sometimes results in “capillary leakage” followed by edema formation (Bartsch, et al., 1991, N. Engl. J. Med., 325: 1284-1289). Human subjects who are susceptible to the disease demonstrate an increased pulmonary vascular response even during a brief exposure of high altitude. The underlying pathophysiological mechanism for this exaggerated HPV is still unknown. There is, however, evidence that the endogenous vasodilator nitric oxide (NO) modulates vascular reactivity (Palmer, et al., 1987, Nature, 327: 524-526). Regulation of vascular tone by NO is attributed to the intermediates of cGMP pathway (Bellamy, et al., 2002, Proc. Nat'l. Acad. Sci. USA, 99: 507-510.). The following studies emphasize the involvement of NO in HAPE:

NO exerts its effect mainly via improvement of ventilation/perfusion ratio and lowering of alveolar to arterial oxygen tension difference by increasing arterial oxygen saturation (Scherrer, et al., 1996, N. Engl. J. Med., 334: 624-629). However, in the healthy volunteers, administration of the NO synthesis antagonist N^{G}-monomethyl-L-arginine (L-NMMA) during hypoxia increases pulmonary artery pressure and vascular resistance which is similar to that observed in HAPE. Due to this observation, NO has been used as an inhalation therapy for the treatment of HAPE in the affected individuals (Anand, et al., 1998, Circulation 98: 2441-2445.). Phosphodiesterase 5 is the key enzyme responsible for cGMP hydrolysis in the lungs. Inhibitors of phosphodiesterase 5 inhibit hypoxia induced pulmonary hypertension (Goldstein, et al., 1998, N. Engl. J. Med., 338: 1397-1404). Hypoxia decreases exhaled NO in mountaineers susceptible to HAPE indicating decreased NO production in such cases (Busch T, et al., 2001, Am. J. Respir. Crit. Care. Med. 163: 368-373). Thus defective NO synthesizing machinery imparting lower NO level may be envisaged to be responsible for the pathogenesis of HAPE. NO is synthesized by three isozymes nNOS (neuronal nitric oxide synthase; NOS1), iNOS (inducible nitric oxide synthase; NOS2) and eNOS (endothelial nitric oxide synthase; NOS3) (Michel, et al., 1997, J. Clin. Invest., 100: 2146-2152.). NOS1 and NOS3 are constitutively expressed while NOS2 is expressed upon induction. Among these enzymes, the best candidate which is supposed to be defective in HAPE is eNOS (endothelial nitric oxide synthase) while induction of iNOS (inducible nitric oxide synthase) seems to be inevitable for the immediate recovery of the total NO reserve (Xia, et al., 1998, J. Biol. Chem., 273: 22635-22639). Moreover, robust cell signaling mechanisms generally favor the recruitment of inducible genes for immediate early physiological responses. It can be speculated that a defect in iNOS which does not permit its activation may not recover the reduced NO level in individuals exposed to hypoxia resulting in HAPE. The defect in iNOS may occur at genetic level in HAPE patients. In numerous cases, the expression of the genes is altered by polymorphisms in the gene sequence (Qadar Pasha, et al., 2001, Ann. Hum. Genet., 65: 531-536). Hence, it is always possible that a polymorphism in the iNOS gene may alter its expression. A variety of therapies are currently used in the treatment of HAPE. NO therapy is used as an inhalation therapy for the treatment of HAPE. It exerts its effect mainly via improvement of the ventilation/perfusion ratio and lowering of alveolar to arterial oxygen tension difference by increasing arterial oxygen saturation. NO induced improvement in arterial oxygenation in subjects with HAPE was accompanied by a shift in blood flow in the lung away from edematous segments and toward nonedematous segments results in evening/homogeneity of the vasoconstriction throughout the capillaries (Scherrer, et al., 1996, N. Engl. J. Med., 334: 624-629; Anand, et al., 1998, Circulation 98: 2441-2445).

Rapid descent of HAPE patients not only prevents exacerbation of HAPE but also improves the pathogenesis of the disease (Hackett, et al., 2001, N. Engl. J. Med., 345: 107-113).

Portable Air Chambers (PACs) in the form of small cylinders filled with oxygen are often used as inhalation therapy for HAPE (Hackett, et al., 2001, N. Engl. J. Med., 345: 107-113).

Genetic predisposition: The only study in this context suggests that genetic variation in endothelial nitric oxide synthase gene (eNOS) and angiotensin converting enzyme gene (ACE) may predispose individuals to HAPE (Droma, et al., 2002, Circulation 106: 826-830). The results are as follows (Table 1):

TABLE 1

| | Controls | Patients |
|---|---|---|
| Glu298Asp (eNOS) | 9.8% | 25.6% |
| B/A (eNOS) | 6.9% | 32.2% |
| I/D (ACE) | 4% | 22% |

Despite a number of therapies for HAPE, all available therapies have limitations. As an example, HAPE patients do not have a homogenous response to NO inhalation. Moreover, concentration of required NO varies with the severity of the disease. Sometimes inadequate inhalation results in hypotension or even septic shock in patients. Further, immediate descent of a HAPE patient often remains impossible due to severe weather and rugged terrain (Anand, et al., 1998, Circulation 98: 2441-2445; Hackett, et al., 2001, N. Engl. J. Med., 345: 107-113). PACs are sometimes not feasible to carry on long treks due to overloading problems and the improvement observed in HAPE patients is often temporary as removal of chambers renders the patient worse (Hackett, et al., 2001, N. Engl. J. Med., 345: 107-113).

The reported polymorphisms associated with HAPE are not specific but have also been shown to be associated with the disorders like diabetes, coronary artery disease, hypertension and myocardial infarction where elevated blood pressure is observed (Monti, et al., 2003, Diabetes, 52: 1270-1275; Via, et al., 2003, Am. J. Med. Genet., 116: 243-248). The allelic frequency difference appears to be the same with other diseases. Hence the possibility of allelic contribution to the disease may be due to other related pathophysiology like hypertension, which exacerbates HAPE. Moreover, the study does not include HA natives (high landers), a population residing in the same environment where the disease occurs.

### SUMMARY OF THE INVENTION

The present invention relates to methods of detecting and predicting of predisposition to HAPE. It particularly relates with the allelic variants of iNOS gene, which has been related to the prevalence of HAPE. Defective Nitric Oxide (NO) synthesizing machinery imparting lower NO level has been discovered to be responsible for the pathogenesis of HAPE. The data disclosed herein demonstrates that the iNOS gene is responsible for NO production as the inhibitors of NO production increased the severity of HAPE. The present invention provides a method for detection of predisposition to HAPE as the novel allelic variants of iNOS gene in the disclosed marker region was shown to be negatively associated with the prevalence of HAPE in a population.

Another embodiment of the present invention provides oligonucleotides primers the for amplification of intron 8 of human iNOS gene and the detection of a predisposition to HAPE.

Another embodiment of the present invention provides allelic variants wherein the allelic variants of the iNOS gene have AA, AG and GG genotypes.

Still another embodiment of the present invention provides a diagnostic kit for the detection of SNP genotypes having predisposition to HAPE.

Yet another embodiment of the present invention provides primers suitable for amplification of an iNOS gene region containing one or more polymorphic sites.

### BRIEF DESCRIPTION OF ACCOMPANYING FIGURES

For the purpose of illustrating the invention, there are depicted in the drawings certain embodiments of the invention. However, the invention is not limited to the precise arrangements and instrumentalities of the embodiments depicted in the drawings.

FIG. 1 is a schematic representation of the gene map of the iNOS gene. Vertical bars indicate the exon regions. The polymorphism is in intron 8 of the iNOS gene, position 87793 of SEQ ID NO:4 or position 19516 of SEQ ID NO:5.

FIG. 2 is an image of a sequencing chromatogram depicting a sequence from an AA homozygote individual.

FIG. 3 is an image of a sequencing chromatograms depicting a sequence from a GG homozygote individual.

FIG. 4 is an image of a sequencing chromatogram depicting a sequence from an AG heterozygote individual.

FIG. 5 is an image of a sequencing chromatogram depicting a sequence from a TC heterozygote individual.

FIG. 6 is a graph depicting the NO level in high altitude dwelling individuals (HA Natives), HAPE controls and HAPE patients.

FIG. 7 is an image of an electrophoresis gel. Lanes 1-3 and 5-8 are 258 by PCR products, lane 4 depicts a 100 bp ladder.

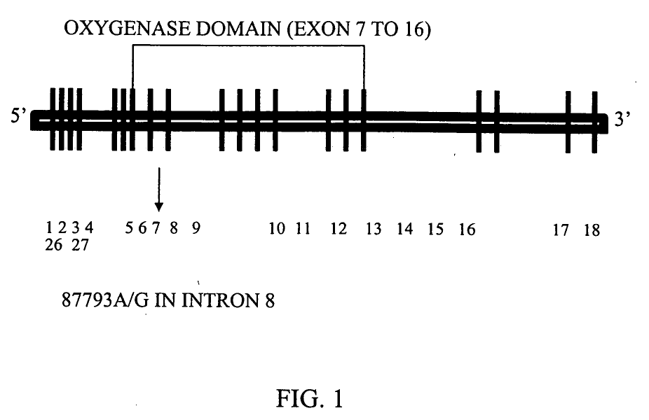

Other and further aspects, features, and advantages of the present invention will be apparent from the following description of the preferred embodiments of the invention given for the purpose of disclosure. Alternative embodiments of the invention can be envisaged by those skilled in the art. All such alternative embodiments are intended to lie within the scope of this invention.

### DETAILED DESCRIPTION OF THE INVENTION

The present invention encompasses methods for detecting a predisposition to high altitude pulmonary edema (HAPE) in a human subject. The method of the present invention comprises identifying a polymorphism in intron 8 of the iNOS gene of a human subject. This is because, as demonstrated by the data disclosed herein, a polymorphism in intron 8 of the iNOS gene in a human subject, specifically a G to A polymorphism, causes a predisposition to HAPE. The polymorphism of the present invention occurs at position 87793 of SEQ ID NO:4. The present polymorphism can also be identified at position 19516 or SEQ ID NO:5, the human iNOS gene. The polymorphism of the present invention can further be identified in the complement of SEQ ID NO:4 at the position complementary to position 87793, or in the complement of SEQ ID NO:5 at the position complementary to position 19516. The sequence of the complement of SEQ ID NO:4 and/or SEQ ID NO:5 will be apparent to one of skill in the art provided with the sequences disclosed herein. The position of the polymorphism of the present invention can be readily identified in the complement of SEQ ID NO:4 and/or SEQ ID NO:5 using the present disclosure, specifically by using the position of the polymorphism in SEQ ID NO:4 and SEQ ID NO:5.

The present invention further encompasses a novel SNP in a human iNOS gene, novel primers and probes for amplification and detection of a predisposition to HAPE. The present invention further encompasses a method of performing an association analysis for an allelic variant between individuals dwelling at lower altitudes and HAPE patients so that the relation with the disease could be scored.

The methods of the present invention further comprise identifying the polymorphism disclosed herein by various methods. Such methods include, but are not limited to, sequencing the iNOS gene from a human subject and amplifying and then sequencing the iNOS gene from a human subject. The present method is not limited to amplifying the entire iNOS gene from a human subject, but rather can encompass amplifying a portion of the iNOS gene comprising the polymorphism disclosed herein. Methods of amplifying and sequencing a gene, or a portion thereof, are disclosed elsewhere herein. Additional methods of identifying a polymorphism include, but are not limited to, hybridizing an allele specific probe to a portion of a gene comprising the polymorphism disclose herein. Methods and compositions for hybridizing a probe, including an allele specific probe, are disclosed elsewhere herein. The present invention further includes isolated polynucleotides for detecting a predisposition to HAPE in a human subject. This is because, as disclosed herein, primers were generated that identified the polymorphism of the present invention, and the polymorphism is useful in the detection of a predisposition to HAPE.

The present invention is useful in the diagnosis, detection of a predisposition and the treatment of HAPE. That is, the present invention is useful in that it identifies the inducible nitric oxide synthase gene as a novel marker for HAPE studies. Further, the present invention discloses novel primer sequences for use in the amplification of a PCR product containing novel SNP related to HAPE. The present invention further comprises a novel SNP that can be used for further studies in determining a predisposition to HAPE in a human subject, and identifies a significant association of to the wild type allele (A) to the disease, a significant association of the mutant allele (G) to adaptation, and a significant difference between the frequency of alleles with respect to HA native and HAPE controls. Specifically, as demonstrated by the data disclosed herein, the presence of the G allele predisposes an individual to less chances of developing HAPE. Further, individuals susceptible to HAPE, as disclosed herein, can prepare for HAPE using methods of treatment disclosed herein and known in the art, thereby decreasing the levels of morbidity and mortality associated with this disease.

The allelic variants of human iNOS gene comprising the following single nucleotide polymorphism was found on comparison with the human iNOS gene sequence, which comprises part of SEQ ID NO:4.

TABLE 1

| Site of change | Base change | Mutation type |
|---|---|---|
| 87793 | A/G | Transition |

The invention also provides a method of analyzing a nucleic acid from an individual for the presence of base at the polymorphic site shown in Table 1. This type of analysis can be performed on a plurality of individuals who are tested either for the presence or for the predisposition to HAPE. The susceptibility to the disease can then be established based depending on the base or set of bases present at the polymorphic site in the individual tested.

The invention also provides oligonucleotide sequences (as listed in SEQ ID NO:2 and SEQ ID NO:3), suitable for use as primers and probes for the detection of the polymorphic site disclosed in Table 1.

The present invention further includes a diagnostic kit comprising one or more primers or probes along with the required buffers and accessories suitable for identification of iNOS allelic variants to establish an individual's susceptibility to HAPE.

The present invention further encompasses vectors comprising a DNA sequence coding for a protein or a peptide according to the invention. A host cell, for example, as well as cloned human cell lines, can be transformed using the new vectors and are also included in the invention.

The present invention relates to the method of detection of predisposition to HAPE. It particularly relates with the allelic variants of iNOS gene, which has been found to be related to the prevalence of HAPE.

I. Identification of the Marker Region on the iNOS Gene:

Taking in consideration the important functions of NO at high altitude, iNOS, the inducible nitric oxide synthase gene was selected as the candidate gene for the study.

### II. Selection of the Study Subjects:

Clinical severity of HAPE was assessed by the Lake Louise acute mountain sickness (AMS) scoring system. Briefly, patients were assessed for the presence of five symptoms: headache, gastrointestinal upset, fatigue, weakness, or both, dizziness, lightheadedness, or both, and difficulty in sleeping. Change in mental status, ataxia and peripheral edema were also assessed. Each of these symptoms were rated between 0 and 3. A score of 0 indicated no symptoms; 1, mild symptoms; 2, moderate symptoms; and 3, severe symptoms. HAPE score is the sum of all 8 symptoms and patients were characterized by HAPE score >6 (Anand, et al., 1998, Circulation 98: 2441-2445). Lowlanders (LLs) were subjects who never demonstrated any of the symptoms of HAPE even after induction to high altitudes at least three times. High altitude (HA) natives were the permanent residents of HA from ancient times.

III. Extraction of Genomic DNA from Leukocytes:

Genomic DNA was extracted from blood using a salting out method. Lysis of red blood cells in presence of high salt was followed by treatment with nucleus lysis buffer (NLB). Proteins were precipitated and extraction of DNA was obtained in ethanol (Miller, et al., 1988, Nucleic Acids Research 16: 1215).

IV. Identification of the Allelic Variants of the iNOS Gene: Novel Polymorphism of the Invention

As a first step to the present invention, a marker region of the iNOS gene was amplified by PCR using self designed oligonucleotide primers. The primers were designed in accordance with the human iNOS gene sequence, which comprises a portion of Gene Bank Accession Number AC130289 (SEQ ID NO:4). A BLAST search of the selected marker region of the present study revealed the homology of with iNOS gene sequence region having with the Homo sapiens chromosome 17, clone RP1-66C13, complete sequence (Gene Bank Accession Number AC130289). It was observed that the NT_{—}010799 denotes the whole chromosome 17 contig, whereas, AC130289 (SEQ ID NO:4) is the fragment of the same contig defining the presence of the sequence in a clone. The sequencing of the purified PCR product revealed a novel single nucleotide polymorphism in intron 8 of the human iNOS gene (SEQ ID NO:5). The position of the polymorphism of the present invention is at position 19516 of SEQ ID NO:5, the human iNOS gene. It was apparent, therefore that there is a hitherto unrecognized allele or subtype of the human iNOS gene.

The present invention provides a sequence for the allelic variants of human iNOS gene comprising the following novel single nucleotide polymorphism compared with the human iNOS gene sequence in the database.

TABLE 2

| Site of change | Base change | Mutation type |
|---|---|---|
| 87793 | A/G | Transition |

For example, the nucleotide sequence of the allelic variant of human iNOS gene (SEQ ID NO:1) having the polymorphic site listed in Table 2 may be—

5′CAGCGGAGTGATGGCAAGCACGACTTCCGGGTGTGGAATGCTCAGCTC
ATCCGCTATGCTGGCTACCAGATGCCAGATGGCAGCATCAGAGGGGACCC
TCGCCAACGTGGAATTCACTCAGGTACCCGGCCCAGCCTCAGCCA/G*CC
GGCCATTGGGGCGGGGAGCCCCGTGGTGAGCGAGTGACAGAGTGGAGCCC
AGAGGAGACACGCAGCCCGGGCTTACAGACTCACAGGGCCCGTCTTGTTC
CCCAGCTGTGCATC3′

In the above sequence the SNP* is indicated by an *.

V. Association Analysis with the Disease

Analysis of the SNP in 42 HA natives, 39 HAPE controls and 18 HAPE patients revealed three genotypes, namely AA, AG and GG. The distribution of alleles is summarized in Table 3.

TABLE 3

| Study subjects | A | G |
|---|---|---|
| HAPE controls (n = 39) | 0.35 | 0.65 |
| HAPE patients (n = 18) | 0.58 | 0.42 |
| HA natives (n = 42) | 0.18 | 0.82 |

The frequency of the G allele was found to be in the order of HA natives>HAPE controls>HAPE subjects. The biostatistical analysis showed a significant association of G allele with HA adaptation and A allele with the disease as disclosed in Table 4.

TABLE 4

| Association type | χ² value | p value | Odds ratio | 95% CI | Relative risk |
|---|---|---|---|---|---|
| HAPE patients & HAPE controls | 10.63 | 0.001 | 2.56 | 1.45-4.54 | 1.66 (1.21-2.27) |
| HAPE patients & HA natives | 33.96 | 0.001 | 6.29 | 3.30-12.01 | 3.22 (2.05-5.06) |
| HAPE controls & HA natives | 7.42 | 0.006 | — | — | — |

Herein the odds ratio (OR) and 95% confidence of interval was used as a measure of the strength of the association between genotypic combination and the disease. P value of <0.05 was considered statistically significant.

Nitric oxide synthase for its reaction to synthesize nitric oxide, requires oxygen which acts as a cofactor in the reaction. Oxygen binds to the oxygenase domain in iNOS and contributes to the synthesis of NO. In hypoxic condition scarcity of oxygen may lead to lower NO production, however any modification in the oxygenase domain, which modify the activity of the enzyme in such a way that it requires no oxygen or less oxygen may contribute to normal NO production. NO improves oxygenation of hemoglobin and normal NO production may involve the mechanisms acting in acclimatization, hence any alteration in oxygenase domain may be favorable for the production of NO. In the present investigation the novel SNP found in intron 8 is present near to the oxygenase domain of NOS2 gene which spans exon 7 to exon 16. While not wishing to be bound by any particular theory, it is quite possible that the SNP found is in linkage disequilibrium to a nearby SNP, which is contributing to the final impact on NO production by NOS2 gene.

### VI. Diagnostic Kits

The invention further provides diagnostic kit comprising at least one or more allele specific oligonucleotides as described in SEQ ID NO:2 and SEQ ID NO:3. The kits of the present invention can comprise one or more pairs of allele-specific oligonucleotides hybridizing to different forms of a polymorphism. In some kits, the allele-specific oligonucleotides are provided immobilized to a substrate. For example, the same substrate can comprise allele-specific oligonucleotide probes for detecting at least the polymorphism shown in Table 1. Optional additional components of the kit include, for example, restriction enzymes, reverse transcriptase or polymerase, the substrate nucleoside triphosphates, means used to label (for example, an avidin enzyme conjugate and enzyme substrate and chromogen if the label is biotin), and the appropriate buffers for reverse transcription, PCR, or hybridization reactions. The kit can also comprise an instructional material for carrying out the methods of the present invention. The instructional material simply describes the embodiments of the invention disclosed herein.

### VII. Nucleic Acid Vectors

Variant genes can be expressed in an expression vector in which a variant gene is operably linked to a native or other promoter. Usually, the promoter is eukaryotic promoter for expression in a mammalian cell. The transcription regulation sequences typically include a heterologous promoter and optionally an enhancer, which is recognized by the host. The selection of an appropriate promoter, for example tip, lac, phage promoters, glycolytic enzyme promoters and tRNA promoters, depends on the host selected. Commercially available expression vectors can also be used. Suitable host cells include bacteria such as E. coli, yeast, filamentous fungi, insect cells, mammalian cells, typically immortalized, e.g., mouse, CHO, human and monkey cell lines and derivatives thereof. Preferred host cells are able to process the variant gene product to produce an appropriate mature polypeptide.

The invention further provides transgenic non-human animals capable of expressing an exogenous variant gene and/or having achieved by operably linking the gene to a promoter and optionally an enhancer, and microinjecting the construct into a zygote. Inactivation of endogenous variant genes can be achieved by forming a transgene in which a cloned variant gene is inactivated by insertion of a positive selection marker. The transgene is then introduced in to an embryonic stem cell, where it undergoes homologous recombination with an endogenous variant gene. Mice and other rodents are preferred animals. Such animals provide useful drug screening systems.

The present invention is described by way of the following examples. The following examples are given by way of illustration of the present invention and should not be construed to limit the scope of the present invention.

### Example 1

### Identification of the Marker Gene

Taking in consideration the important functions of NO at HA, iNOS, the inducible nitric oxide synthase was selected as the candidate gene for the study.

Extraction of Genomic DNA from Leukocytes:

Genomic DNA was extracted from blood using salting out method. Lysis of red blood cells in presence of high salt was followed by treatment with nucleus lysis buffer (NLB). Proteins were precipitated and DNA was extracted from peripheral blood leukocytes using a modification of the salting out procedure. The concentration of the DNA was determined by measuring the optical density of the sample, at a wavelength of 260 nm. (Miller, et al., 1988, 16: 1215).

### Example 2

### Selection of the Study Subjects

Clinical severity of HAPE was assessed by Lake Louise acute mountain sickness (AMS) scoring system. Briefly, the patients were assessed for the presence of five symptoms: headache, gastrointestinal upset, fatigue, weakness, or both, dizziness, lightheadedness, or both, and difficulty in sleeping. Change in mental status, ataxia and peripheral edema were also assessed. Each of these symptoms were rated between 0 and 3. A score of 0 indicated no symptoms; 1, mild symptoms; 2, moderate symptoms; and 3, severe symptoms. HAPE score is the sum of all 8 symptoms and patients were characterized by HAPE score >6 (Anand, et al., 1998, Circulation 98: 2441-2445). Lowlanders (LLs) were subjects who never demonstrated any of the symptoms of HAPE even after induction to high altitudes at least three times. High altitude (HA) natives were the permanent residents of HA from ancient times.

### Example 3

### Identification of the Allelic Variants of the iNOS Gene

This example describes the identification of allelic variants of iNOS gene by PCR and sequencing using certain chemically synthesized oligonucleotide primers according to the invention. The DNA was then amplified by polymerase chain reaction by using the oligonucleotide primers:

| | |
|---|---|
| 1. 5′CAGCGGAGTGATGGCAAGCACGAC 3′ | (SEQ ID NO: 2) |
| and | |
| 2. 5′ GATGCACAGCTGGGGAACAAGACG 3′. | (SEQ ID NO: 3) |

Polymerase chain reaction was carried out using the following conditions:

Step 1 94° C. for 4 minutes;
Step 2 94° C. for 30 seconds
Step 3 62.5° C. for 30 seconds
Step 4 72° C. for 45 seconds
Step 5 34 times to Step 2
Step 6 72° C. for 10 minutes

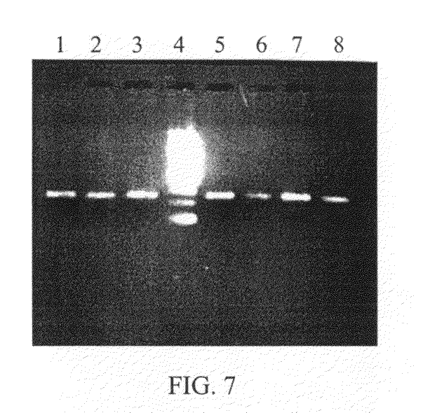

PCR was performed in a Perkin Elmer GeneAmp PCR System 9600. This reaction produced a DNA fragment of 258 bp when analyzed by 2% agarose gel electrophoresis (FIG. 7). The PCR product was purified from band by excision from the agarose gel using a Amersham Pharmacia gel extraction kit (Amersham) and both the strands of the PCR product were directly sequenced using dye terminator chemistry on an ABI Prism 377 automated DNA sequencer. The PCR product was identical to the human iNOS gene sequence except of the novel single base pair change disclosed in Table 1.

### Example 4

### Nucleotide Sequence of the Allelic Variant of the iNOS Gene

The nucleotide sequence of the allelic variant of iNOS gene derived using the method as described in example 1 (SEQ ID NO:1)

5′CAG CGG AGT GAT GGC AAG CAC GAC TTC CGG GTG TGG
AAT GCT CAG CTC ATC CGC TAT GCT GGC TAC CAG ATG
CCA GAT GGC AGC ATC AGA GGG GAC CCT GCC AAC GTG
GAA TTC ACT CAG GTA CCC GGC CCA GCC TCA GCC A/G*CC
GGC CAT TGG GGC GGG GAG CCC CGT GGT GAG CGA GTG
ACA GAG TGG AGC CCA GAG GAG ACA CGC AGC CCG GGC
TTA CAG ACT CAC AGG GCC CGT CTT GTT CCC CAG CTG
TGC ATC 3′

In the above sequence the SNP is indicated by an *.

The intron/exon junction sequences for at least the junctions flanking the SNP are the following:

(SEQ ID NO: 6)

gtacccggcccagcctcagcca/g*ccggccattggggcggggagccccg
tggtgagcgagtgacagagtggagcccagaggagacacgcagcccgggct
tacagactcacagggcccgtcttgttccccag

Important ten nucleotides of intron 8 towards the upstream and downstream of the SNP. The 5′ gt and the 3′ ag are the 5′ acceptor and 3′ donor sites, respectively.

### Example 5

G allele is related with adaptation and A allele associates with the disease: A method as described in example 4 is applied to a series of DNA samples extracted from HA natives, HAPE controls and HAPE patients. A highly significant association of G allele with the HA adaptation and A allele with the disease has been observed. The results are summarized in Table 5 below:

TABLE 5

| Association type | χ² value | p value | Odds ratio | 95% CI | Relative risk |
|---|---|---|---|---|---|
| HAPE patients & HAPE controls | 10.63 | 0.001 | 2.56 | 1.45-4.54 | 1.66 (1.21-2.27) |
| HAPE patients & HA natives | 33.96 | <0.001 | 6.29 | 3.30-12.01 | 3.22 (2.05-5.06) |
| HAPE controls & HA natives | 7.42 | 0.006 | — | — | — |

Hence, the individuals with AG genotype will be at less risk of HAPE as compared to the AA and more at risk as compared to GG.

Hence, individuals with GG genotype being at low risk and those with AA genotype being at high risk for HAPE, can be expected to hold true for other populations also.

### Example 6

### Experimental Methodology

Isolation of Genomic DNA from Blood Samples

### Buffers Preparation and Composition:

a) Cell lysis buffer
1.28M Sucrose
1M Tris-HCl (pH 7.5)
20 mM MgCl₂
4% Triton X-100

Dissolve the contents in a final volume of 400 ml of warm distilled water. Autoclave and store at 4° C.

b) Nuclear lysis buffer (NLB)
1M Tris-HCl (pH 8.0)
5M NaCl
0.5M EDTA (pH 8.0)

Dissolve in a final volume of 400 ml of distilled water. Autoclave, cool and store at 4° C.

c) 50×TE buffer (1 liter)
2M Tris-HCl (pH 8.0)
0.5M EDTA 100 ml (pH 8.0)
   d) 50×TAE Buffer (1 liter)
2M Tris-HCl (pH 8.0) 242 g
Glacial acetic acid: 57.1 ml
0.5M EDTA (pH 8.0): 100 ml

Procedure of Isolation:
1) Transfer 3 to 5 ml human bloods in ACD to centrifuge tubes. Label the tubes appropriately.
2) Add 20 ml lysis buffer (5 ml blood+5 ml 4× lysis buffer+15 ml water), shake well and keep in ice for 10 minutes.
3) Centrifuge at 3300 rpm for 20 minutes at 4° C. Discard the supernatant in sodium thiosulphate solution.
4) Add 5 ml 1×-lysis buffer. Vortex the contents gently and centrifuge at 3300 rpm for 15 minutes at 4° C. Decant the supernatant and check for the clarity of the pellet.
   5) Add 5 ml 1×-lysis buffer after gentle vortexing. Centrifuge as above for 10 minutes and retain a clear white pellet (WBCs).
6) Add 6 ml NLB for the lysis of nuclear membranes, 400 μl 10% SDS and 25 μl Proteinase K (10 mg/ml). Vortex the contents to suspend the pellet.
7) Incubate at 65° C. for 2-2.5 hours with continuous stirring in a shaker incubator.
8) Add 2 ml of saturated NaCl (6M). Shake gently and centrifuge for 15 minutes at 2500 rpm at 37° C.
9) Very carefully decant the clear supernatant in another sterile tube and add 4 volumes of absolute alcohol. The tube was rotated gently in slow motion. White threads of DNA will appear at the junction of the two solutions.
10) Transfer the spools of DNA to 1 ml of chilled 70% ethanol.
11) Centrifuge at 10,000 rpm for 15 minutes and dry the pellet at room temperature.
12) Dissolve the DNA pellet in 400 μl of TE buffer at 65° C. for 2 hours in a dry bath.
13) Dilute each of the DNA samples 100 times (10 μl DNA+990 μl autoclaved TDW) and read the absorbance at 260 and 280 nm in a UV-VIS spectrophotometer. Calculate the ratio of DNA to protein to check the purity of the DNA samples.
14) Calculate the concentration of DNA from the OD at 260 nm. (1 OD unit corresponds to 50 μg/ml of ds DNA).
15) Dilute the DNA samples to 25 ng/μl with 1×TE buffer accordingly and check the concentration on 0.8% agarose gel.

### Purification of the PCR Product

Purification of the PCR product was done by Poly Ethyl Glycol (PEG) method

1. Add two volume of PEG in to the PCR product.
2. Incubate it for ten minutes at room temperature.
3. Centrifuge at 3200 rpm for one hour.
4. Add two volumes of 70% ethyl alcohol and spin for ten minutes at 3200 rpm at room temperature.
5. Remove ethyl alcohol by inverting the tube and rotate it up to the 500 rpm.
6. Repeats the steps 4 and 5 and dry it at least up to one hour then dissolved it in to the 12 μl Elix water.
7. Check it in to the gel and use this DNA as template for the sequencing PCR.

### Protocol for the Sequencing PCR

Protocol for 10 μl cycles sequencing PCR for three reactions
1. Reaction mixture (RM)=6.0 μl
2. Dilution Buffer (DB)=3.0 μl
3. Primer (forward or reverse)=6.0 μl
4. This mixture of three solutions called as CRM. Transfer this CRM into three tubes.
5. Add template=5 μl in each tube and make a total volume of 10 μl.
   The Sequencing PCR Conditions were

| | |
|---|---|
| Denaturation | at 96° C. for 10 seconds |
| Primer Annealing and Extension | at 60° C. for 4 minutes |
| Hold | at 4° C. |
| No of cycles | 25 Cycles |

### Purification of the Sequencing Product

1) Add 26 μl MQ (sterile) water to 10 μl PCR product.
2) Mix well and transfer to 0.6 μl Eppendorf.
3) Add 64 μl absolute alcohol (chilled) and mix thoroughly.
4) Incubate it at room temperature for 10 minutes.
5) Centrifuge at 16000 g for 20 minutes at 25° C.
6) Decant add 150 μl 170% ethyl alcohol.
7) Centrifuge at 16,000 g for 5 minutes at 20° C.
8) Decant and repeat step 6.
9) Aspirate by pipette taking care for not touching the well.
10) Air-dry the pipette.

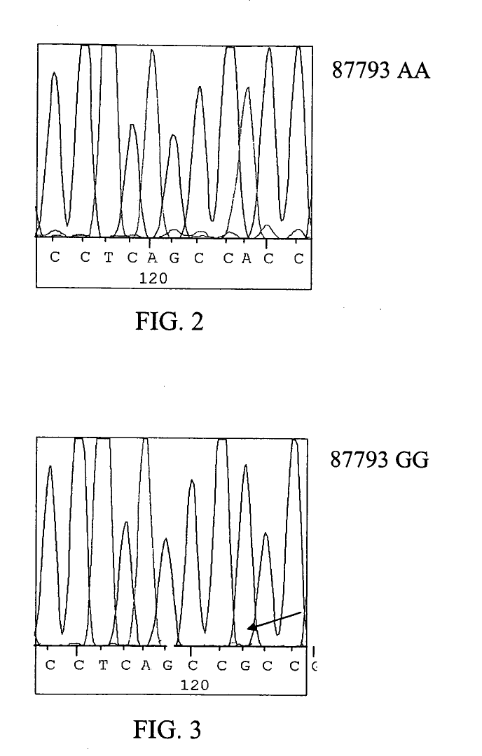

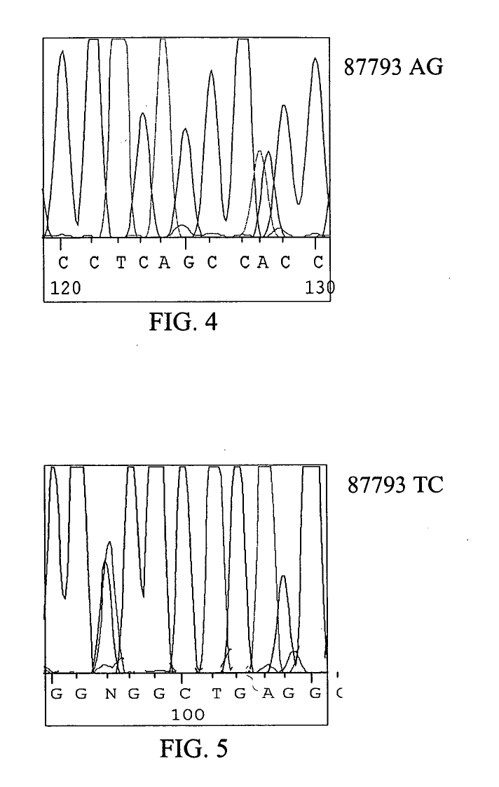

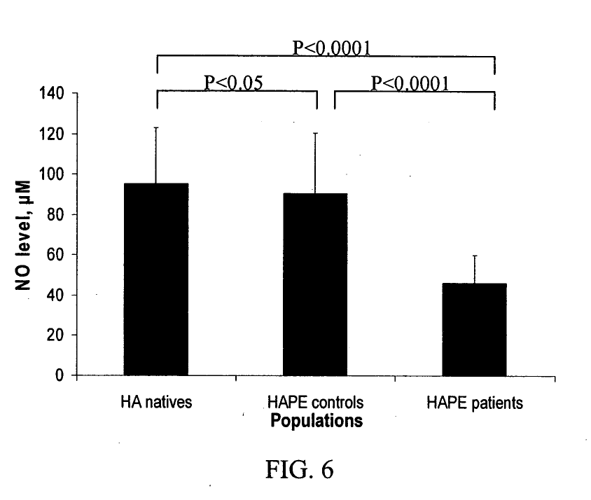

Purified products were sequenced using both forward and backward primers by ABI prism 377 sequencer. The portions of the sequencing chromatograms containing both the alleles of the SNP are depicted in FIGS. 2 through 5.

The designed pair of primers SEQ ID NO:2 and SEQ ID NO:3 by the PRIMER SELECT of DNAstar are presented below in Table 6:

| | |
|---|---|
| Upper Primer | 5′CAGCGGAGTGATGGCAAGCACGAC 3′ |
| (SEQ ID NO: 2) | |
| Lower Primer | 5′ GATGCACAGCTGGGGAACAAGACG 3′ |
| (SEQ ID NO: 3) | |

| DNA 250 pM, | | |
|---|---|---|
| Salt 50 mM | Upper Primer | Lower Primer |
| Primer Tm | 66.4° C. | 63.5° C. |
| Primer Overall | −49.2 kc/m | −47.5 kc/m |
| Stability | | |
| Primer | 19375 . . . 19398 | 19632 . . . 19609 |
| Location | | |

| | |
|---|---|
| Product Tm- | 19.8° C. |
| Primer Tm | |
| Primers Tm | 2.9° C. |
| Difference | |
| Optimal | 62.5° C. |
| Annealing | |
| Temperature | |
| Product Length | 258 bp |
| Product Tm (% | 83.3° C. |
| GC Method) | |
| Product GC | 63.6% |
| Content | |
| Product Tm at | 104.9° C. |
| 6xSSC | |

Product Melting Temperature (% GC Method)

| Salt | | | Formamide | | | |
|---|---|---|---|---|---|---|
| mM | xSSC | xSSPE | 0% | 10% | 20% | 50% |
| 1 | 0.005 | 0.006 | 55.1 | 48.6 | 42.1 | 22.6 |
| 10 | 0.051 | 0.062 | 71.7 | 65.2 | 58.7 | 39.2 |
| 50 | 0.256 | 0.312 | 83.3 | 76.8 | 70.3 | 50.8 |
| 165 | 0.846 | 1.031 | 92.0 | 85.5 | 79.0 | 59.5 |
| 330 | 1.692 | 2.062 | 97.0 | 90.5 | 84.0 | 64.5 |
| 500 | 2.564 | 3.125 | 99.9 | 93.4 | 86.9 | 67.4 |
| 1000 | 5.128 | 6.250 | 104.9 | 98.4 | 91.9 | 72.4 |

| | | | |
|---|---|---|---|
| 195 | 1.000 | 1.219 | +0.0% formamide = |
| | | | Tm 104.9° C. |

The PCR products were loaded on Agarose gel electrophoresis and visualized by ethidium bromide staining. The size of the 258 by products was confirmed by the molecular weight marker (FIG. 7).

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method for detecting predisposition to high altitude pulmonary edema (HAPE) in a human subject, said method comprising identifying a polymorphism in intron 8 of an inducible nitric oxide synthase gene of said subject, wherein said polymorphism is at position 19,516 in SEQ ID NO: 5, wherein predisposition to HAPE is indicated by the presence of the nucleotide A at said position.

2. The method of claim 1, wherein the nucleotide identification comprises sequencing a region of said subject's genomic DNA, wherein said region comprises said position.

3. The method of claim 1, wherein the nucleotide identification comprises a) amplifying a portion of said subject's genomic DNA comprising said position, and b) sequencing the amplified portion of said subject's genomic DNA.

4. The method of claim 3, wherein said amplification is by polymerase chain reaction.

5. The method of claim 4, wherein amplification comprises: (a) hybridizing a forward primer under polymerase chain reaction conditions to a sequence upstream of said position, said forward primer comprising a sequence that hybridizes to the complement of SEQ ID NO:2 under polymerase chain reaction conditions; and (b) hybridizing a reverse primer under polymerase chain reaction conditions to a sequence downstream of said position, said reverse primer comprising a sequence that hybridizes to the complement of SEQ ID NO:3 under polymerase chain reaction conditions.

6. The method of claim 4, wherein amplification comprises: (a) hybridizing a forward primer comprising the sequence set forth in SEQ ID NO:2 under polymerase chain reaction conditions to a sequence upstream of said position; and (b) hybridizing a reverse primer comprising the sequence set forth in SEQ ID NO:3 under polymerase chain reaction conditions to a sequence downstream of said position.

7. The method of claim 1, comprising hybridizing an allele specific probe to DNA selected from the group consisting of a portion of said subjects genomic DNA comprising said position, and amplified DNA from the genomic DNA of said subject, wherein said genomic DNA comprises said position.

8. The method of claim 7, said allele specific probe selected from the group consisting of a probe that hybridizes to said DNA when said nucleotide is A, and a probe that hybridizes to said DNA when said nucleotide is G.

9-14. (canceled)

15. A method for detecting a predisposition to high altitude pulmonary edema (HAPE) in a human subject comprising:
i) identifying a genotype associated with a polymorphism at position 19,516 of WES ID NO: 5, wherein genotype AA is associated with a high risk of HAPE and genotype GG is associated with a low risk of HAPE, and
ii) detecting said predisposition to HAPE by identifying said genotype.

16. A method as claimed in claim 1 wherein the nucleotide identification comprises sequencing PCR amplified region of said subject's genomic DNA, wherein said region comprises said position.

17. A method as claimed in claim 15 wherein the nucleotide identification comprises sequencing PCR amplified region of said subject's genomic DNA, wherein said region comprises said position
